SEQUENCE LISTING (243 sequences)

SEQ ID NO: 1 - protein, 5 aa, synthetic construct
  REGION 1..5: note = ANT4044 Variable Heavy chain - HCDR1 (protein)
EYTIH

SEQ ID NO: 2 - protein, 17 aa, synthetic construct
  REGION 1..17: note = ANT4044 Variable Heavy chain - HCDR2 (protein)
NINPNNGGTTYNQKFED

SEQ ID NO: 3 - protein, 6 aa, synthetic construct
  REGION 1..6: note = ANT4044 Variable Heavy chain - HCDR3 (protein)
GWNFDY

SEQ ID NO: 4 - protein, 115 aa, synthetic construct
  REGION 1..115: note = ANT4044 Variable Heavy chain - VH (protein)
EVQLVQSGAEVKKPGASVKVSCKASGYTFTEYTIHWVRQAPGKGLEWIGNINPNNGGTTYNQKFEDRVTITVDKSTSTAYMELSSLRSEDTAVYYCAAGWNFDYWGQGTTVTVSS

SEQ ID NO: 5 - DNA, 15 bp, synthetic construct
  misc_feature 1..15: note = ANT4044 Variable Heavy chain - HCDR1 (DNA)
  source 1..15: mol_type = other DNA
gaatacaccatccac

SEQ ID NO: 6 - DNA, 51 bp, synthetic construct
  misc_feature 1..51: note = ANT4044 Variable Heavy chain - HCDR2 (DNA)
  source 1..51: mol_type = other DNA
aacattaatcctaacaatggtggtactacctacaaccagaagttcgaggac

SEQ ID NO: 7 - DNA, 18 bp, synthetic construct
  misc_feature 1..18: note = ANT4044 Variable Heavy chain - HCDR3 (DNA)
  source 1..18: mol_type = other DNA
ggttggaactttgactac

SEQ ID NO: 8 - DNA, 345 bp, synthetic construct
  misc_feature 1..345: note = ANT4044 Variable Heavy chain - VH (DNA)
  source 1..345: mol_type = other DNA
gaggtccagctggtgcagtctggagctgaggtgaagaagcctggggcctcagtgaaggtctcctgcaaggcttctggatacacattcactgaatacaccatccactgggtgaggcaggcccctggaaagggccttgagtggattggaaacattaatcctaacaatggtggtactacctacaaccagaagttcgaggacagagtcacaatcactgtagacaagtccaccagcacagcctacatggagctcagcagcctgagatctgaggatactgcagtctattactgtgcagctggttggaactttgactactggggccaaggcaccacggtcaccgtctcctca

SEQ ID NO: 9 - protein, 30 aa, synthetic construct
  REGION 1..30: note = ANT4044 Variable Heavy chain - HFR1 (protein)
EVQLVQSGAEVKKPGASVKVSCKASGYTFT

SEQ ID NO: 10 - protein, 14 aa, synthetic construct
  REGION 1..14: note = ANT4044 Variable Heavy chain - HFR2 (protein)
WVRQAPGKGLEWIG

SEQ ID NO: 11 - protein, 32 aa, synthetic construct
  REGION 1..32: note = ANT4044 Variable Heavy chain - HFR3 (protein)
RVTITVDKSTSTAYMELSSLRSEDTAVYYCAA

SEQ ID NO: 12 - protein, 11 aa, synthetic construct
  REGION 1..11: note = ANT4044 Variable Heavy chain - HFR4 (protein)
WGQGTTVTVSS

SEQ ID NO: 13 - DNA, 90 bp, synthetic construct
  misc_feature 1..90: note = ANT4044 Variable Heavy chain - HFR1 (DNA)
  source 1..90: mol_type = other DNA
gaggtccagctggtgcagtctggagctgaggtgaagaagcctggggcctcagtgaaggtctcctgcaaggcttctggatacacattcact

SEQ ID NO: 14 - DNA, 42 bp, synthetic construct
  misc_feature 1..42: note = ANT4044 Variable Heavy chain - HFR2 (DNA)
  source 1..42: mol_type = other DNA
tgggtgaggcaggcccctggaaagggccttgagtggattgga

SEQ ID NO: 15 - DNA, 96 bp, synthetic construct
  misc_feature 1..96: note = ANT4044 Variable Heavy chain - HFR3 (DNA)
  source 1..96: mol_type = other DNA
agagtcacaatcactgtagacaagtccaccagcacagcctacatggagctcagcagcctgagatctgaggatactgcagtctattactgtgcagct

SEQ ID NO: 16 - DNA, 33 bp, synthetic construct
  misc_feature 1..33: note = ANT4044 Variable Heavy chain - HFR4 (DNA)
  source 1..33: mol_type = other DNA
tggggccaaggcaccacggtcaccgtctcctca

SEQ ID NO: 17 - protein, 5 aa, synthetic construct
  REGION 1..5: note = ANT4044-A2 Variable Heavy chain - HCDR1 (protein)
EYTIH

SEQ ID NO: 18 - protein, 17 aa, synthetic construct
  REGION 1..17: note = ANT4044-A2 Variable Heavy chain - HCDR2 (protein)
NINPNNGGTTYNQKFED

SEQ ID NO: 19 - protein, 6 aa, synthetic construct
  REGION 1..6: note = ANT4044-A2 Variable Heavy chain - HCDR3 (protein)
YWLFDY

SEQ ID NO: 20 - protein, 115 aa, synthetic construct
  REGION 1..115: note = ANT4044-A2 Variable Heavy chain - VH (protein)
EVQLVQSGAEVKKPGASVKVSCKASGYTFTEYTIHWVRQAPGKGLEWIGNINPNNGGTTYNQKFEDRVTITVDKSTSTAYMELSSLRSEDTAVYYCAAYWLFDYWGQGTTVTVSS

SEQ ID NO: 21 - DNA, 15 bp, synthetic construct
  misc_feature 1..15: note = ANT4044-A2 Variable Heavy chain - HCDR1 (DNA)
  source 1..15: mol_type = other DNA
gaatacaccatccac

SEQ ID NO: 22 - DNA, 51 bp, synthetic construct; identical to SEQ ID NO: 6
  misc_feature 1..51: note = ANT4044-A2 Variable Heavy chain - HCDR2 (DNA)
  source 1..51: mol_type = other DNA

SEQ ID NO: 23 - DNA, 18 bp, synthetic construct
  misc_feature 1..18: note = ANT4044-A2 Variable Heavy chain - HCDR3 (DNA)
  source 1..18: mol_type = other DNA
tactggctgttcgactac

SEQ ID NO: 24 - DNA, 345 bp, synthetic construct
  misc_feature 1..345: note = ANT4044-A2 Variable Heavy chain - VH (DNA)
  source 1..345: mol_type = other DNA
gaggtccagctggtgcagtctggagctgaggtgaagaagcctggggcctcagtgaaggtctcctgcaaggcttctggatacacattcactgaatacaccatccactgggtgaggcaggcccctggaaagggccttgagtggattggaaacattaatcctaacaatggtggtactacctacaaccagaagttcgaggacagagtcacaatcactgtagacaagtccaccagcacagcctacatggagctcagcagcctgagatctgaggatactgcagtctattactgtgcagcttactggctgttcgactactggggccaaggcaccacggtcaccgtctcctca

SEQ ID NO: 25 - protein, 30 aa, synthetic construct; identical to SEQ ID NO: 9
  REGION 1..30: note = ANT4044-A2 Variable Heavy chain - HFR1 (protein)

SEQ ID NO: 26 - protein, 14 aa, synthetic construct
  REGION 1..14: note = ANT4044-A2 Variable Heavy chain - HFR2 (protein)
WVRQAPGKGLEWIG

SEQ ID NO: 27 - protein, 32 aa, synthetic construct; identical to SEQ ID NO: 11
  REGION 1..32: note = ANT4044-A2 Variable Heavy chain - HFR3 (protein)

SEQ ID NO: 28 - protein, 11 aa, synthetic construct
  REGION 1..11: note = ANT4044-A2 Variable Heavy chain - HFR4 (protein)
WGQGTTVTVSS

SEQ ID NO: 29 - DNA, 90 bp, synthetic construct; identical to SEQ ID NO: 13
  misc_feature 1..90: note = ANT4044-A2 Variable Heavy chain - HFR1 (DNA)
  source 1..90: mol_type = other DNA

SEQ ID NO: 30 - DNA, 42 bp, synthetic construct; identical to SEQ ID NO: 14
  misc_feature 1..42: note = ANT4044-A2 Variable Heavy chain - HFR2 (DNA)
  source 1..42: mol_type = other DNA

SEQ ID NO: 31 - DNA, 96 bp, synthetic construct; identical to SEQ ID NO: 15
  misc_feature 1..96: note = ANT4044-A2 Variable Heavy chain - HFR3 (DNA)
  source 1..96: mol_type = other DNA

SEQ ID NO: 32 - DNA, 33 bp, synthetic construct; identical to SEQ ID NO: 16
  misc_feature 1..33: note = ANT4044-A2 Variable Heavy chain - HFR4 (DNA)
  source 1..33: mol_type = other DNA

SEQ ID NO: 33 - protein, 11 aa, synthetic construct
  REGION 1..11: note = ANT4044/ ANT4044-A2 Variable Light chain - LCDR1 (protein)
KASQDVGTAVD

SEQ ID NO: 34 - protein, 7 aa, synthetic construct
  REGION 1..7: note = ANT4044/ ANT4044-A2 Variable Light chain - LCDR2 (protein)
WASTRHT

SEQ ID NO: 35 - protein, 9 aa, synthetic construct
  REGION 1..9: note = ANT4044/ ANT4044-A2 Variable Light chain - LCDR3 (protein)
QQYNSYPLT

SEQ ID NO: 36 - protein, 107 aa, synthetic construct
  REGION 1..107: note = ANT4044/ ANT4044-A2 Variable Light chain - VL (protein)
DIQMTQSPSTLSASVGDRVTITCKASQDVGTAVDWYQQKPGQAPKLLIYWASTRHTGVPDRFSGSGSGTDFTLTISRLQPEDFAVYYCQQYNSYPLTFGQGTKVDIK

SEQ ID NO: 37 - DNA, 33 bp, synthetic construct
  misc_feature 1..33: note = ANT4044/ ANT4044-A2 Variable Light chain - LCDR1 (DNA)
  source 1..33: mol_type = other DNA
aaggccagtcaggatgtgggtactgctgtagac

SEQ ID NO: 38 - DNA, 21 bp, synthetic construct
  misc_feature 1..21: note = ANT4044/ ANT4044-A2 Variable Light chain - LCDR2 (DNA)
  source 1..21: mol_type = other DNA
tgggcatccacccggcacact

SEQ ID NO: 39 - DNA, 27 bp, synthetic construct
  misc_feature 1..27: note = ANT4044/ ANT4044-A2 Variable Light chain - LCDR3 (DNA)
  source 1..27: mol_type = other DNA
cagcaatataacagctatcctctcacg

SEQ ID NO: 40 - DNA, 321 bp, synthetic construct
  misc_feature 1..321: note = ANT4044/ ANT4044-A2 Variable Light chain - VL (DNA)
  source 1..321: mol_type = other DNA
gacattcagatgacccagtctcccagcaccctgtccgcatcagtaggagacagggtcaccatcacttgcaaggccagtcaggatgtgggtactgctgtagactggtatcaacagaaaccagggcaagctcctaaactactgatttactgggcatccacccggcacactggagtccctgatcgcttcagcggcagtggatctgggacagatttcactctcaccatcagcagactgcagcctgaagactttgcagtttattactgtcagcaatataacagctatcctctcacgttcggccaggggaccaaggtggatatcaaa

SEQ ID NO: 41 - protein, 23 aa, synthetic construct
  REGION 1..23: note = ANT4044/ ANT4044-A2 Variable Light chain - LFR1 (protein)
DIQMTQSPSTLSASVGDRVTITC

SEQ ID NO: 42 - protein, 15 aa, synthetic construct
  REGION 1..15: note = ANT4044/ ANT4044-A2 Variable Light chain - LFR2 (protein)
WYQQKPGQAPKLLIY

SEQ ID NO: 43 - protein, 32 aa, synthetic construct
  REGION 1..32: note = ANT4044/ ANT4044-A2 Variable Light chain - LFR3 (protein)
GVPDRFSGSGSGTDFTLTISRLQPEDFAVYYC

SEQ ID NO: 44 - protein, 10 aa, synthetic construct
  REGION 1..10: note = ANT4044/ ANT4044-A2 Variable Light chain - LFR4 (protein)
FGQGTKVDIK

SEQ ID NO: 45 - DNA, 69 bp, synthetic construct
  misc_feature 1..69: note = ANT4044/ ANT4044-A2 Variable Light chain - LFR1 (DNA)
  source 1..69: mol_type = other DNA
gacattcagatgacccagtctcccagcaccctgtccgcatcagtaggagacagggtcaccatcacttgc

SEQ ID NO: 46 - DNA, 45 bp, synthetic construct
  misc_feature 1..45: note = ANT4044/ ANT4044-A2 Variable Light chain - LFR2(DNA)
  source 1..45: mol_type = other DNA
tggtatcaacagaaaccagggcaagctcctaaactactgatttac

SEQ ID NO: 47 - DNA, 96 bp, synthetic construct
  misc_feature 1..96: note = ANT4044/ ANT4044-A2 Variable Light chain - LFR3 (DNA)
  source 1..96: mol_type = other DNA
ggagtccctgatcgcttcagcggcagtggatctgggacagatttcactctcaccatcagcagactgcagcctgaagactttgcagtttattactgt

SEQ ID NO: 48 - DNA, 30 bp, synthetic construct
  misc_feature 1..30: note = ANT4044/ ANT4044-A2 Variable Light chain - LFR4 (DNA)
  source 1..30: mol_type = other DNA
ttcggccaggggaccaaggtggatatcaaa

SEQ ID NO: 49 - protein, 5 aa, synthetic construct
  REGION 1..5: note = Girentuximab chimeric VHO Variable Heavy chain - HCDR1 (protein)
NYYMS

SEQ ID NO: 50 - protein, 17 aa, synthetic construct
  REGION 1..17: note = Girentuximab chimeric VHO Variable Heavy chain - HCDR2 (protein)
AINSDGGITYYLDTVKG

SEQ ID NO: 51 - protein, 10 aa, synthetic construct
  REGION 1..10: note = Girentuximab chimeric VHO Variable Heavy chain - HCDR3 (protein)
HRSGYFSMDY

SEQ ID NO: 52 - protein, 119 aa, synthetic construct
  REGION 1..119: note = Girentuximab chimeric VHO Variable Heavy chain - VH (protein)
DVKLVESGGGLVKLGGSLKLSCAASGFTFSNYYMSWVRQTPEKRLELVAAINSDGGITYYLDTVKGRFTISRDNAKNTLYLQMSSLKSEDTALFYCARHRSGYFSMDYWGQGTSVTVSS

SEQ ID NO: 53 - DNA, 15 bp, synthetic construct
  misc_feature 1..15: note = Girentuximab chimeric VHO Variable Heavy chain - HCDR1 (DNA)
  source 1..15: mol_type = other DNA
aactattacatgtct

SEQ ID NO: 54 - DNA, 51 bp, synthetic construct
  misc_feature 1..51: note = Girentuximab chimeric VHO Variable Heavy chain - HCDR2 (DNA)
  source 1..51: mol_type = other DNA
gccattaatagtgatggtggtatcacctactatctagacactgtgaagggc

SEQ ID NO: 55 - DNA, 30 bp, synthetic construct
  misc_feature 1..30: note = Girentuximab chimeric VHO Variable Heavy chain - HCDR3 (DNA)
  source 1..30: mol_type = other DNA
caccgctcgggctacttttctatggactac

SEQ ID NO: 56 - DNA, 357 bp, synthetic construct
  misc_feature 1..357: note = Girentuximab chimeric VHO Variable Heavy chain - VH (DNA)
  source 1..357: mol_type = other DNA
gacgtgaagctcgtggagtctgggggaggcttagtgaagcttggaggatccctgaaactctcctgtgcagcctctggattcactttcagtaactattacatgtcttgggttcgccagactccagagaagaggctggagttggtcgcagccattaatagtgatggtggtatcacctactatctagacactgtgaagggccgattcaccatttcaagagacaatgccaagaacaccctgtacctgcaaatgagcagtctgaagtctgaggacacagccttgttttactgtgcaagacaccgctcgggctacttttctatggactactggggtcaaggaacctcagtcaccgtctcctca

SEQ ID NO: 57 - protein, 30 aa, synthetic construct
  REGION 1..30: note = Girentuximab chimeric VHO Variable Heavy chain - HFR1 (protein)
DVKLVESGGGLVKLGGSLKLSCAASGFTFS

SEQ ID NO: 58 - protein, 14 aa, synthetic construct
  REGION 1..14: note = Girentuximab chimeric VHO Variable Heavy chain - HFR2 (protein)
WVRQTPEKRLELVA

SEQ ID NO: 59 - protein, 32 aa, synthetic construct
  REGION 1..32: note = Girentuximab chimeric VHO Variable Heavy chain - HFR3 (protein)
RFTISRDNAKNTLYLQMSSLKSEDTALFYCAR

SEQ ID NO: 60 - protein, 11 aa, synthetic construct
  REGION 1..11: note = Girentuximab chimeric VHO Variable Heavy chain - HFR4 (protein)
WGQGTSVTVSS

SEQ ID NO: 61 - DNA, 90 bp, synthetic construct
  misc_feature 1..90: note = Girentuximab chimeric VHO Variable Heavy chain - HFR1 (DNA)
  source 1..90: mol_type = other DNA
gacgtgaagctcgtggagtctgggggaggcttagtgaagcttggaggatccctgaaactctcctgtgcagcctctggattcactttcagt

SEQ ID NO: 62 - DNA, 42 bp, synthetic construct
  misc_feature 1..42: note = Girentuximab chimeric VHO Variable Heavy chain - HFR2 (DNA)
  source 1..42: mol_type = other DNA
tgggttcgccagactccagagaagaggctggagttggtcgca

SEQ ID NO: 63 - DNA, 96 bp, synthetic construct
  misc_feature 1..96: note = Girentuximab chimeric VHO Variable Heavy chain - HFR3 (DNA)
  source 1..96: mol_type = other DNA
cgattcaccatttcaagagacaatgccaagaacaccctgtacctgcaaatgagcagtctgaagtctgaggacacagccttgttttactgtgcaaga

SEQ ID NO: 64 - DNA, 33 bp, synthetic construct
  misc_feature 1..33: note = Girentuximab chimeric VHO Variable Heavy chain - HFR4 (DNA)
  source 1..33: mol_type = other DNA
tggggtcaaggaacctcagtcaccgtctcctca

SEQ ID NO: 65 - protein, 5 aa, synthetic construct
  REGION 1..5: note = Girentuximab humanised VH1 Variable Heavy chain - HCDR1 (protein)
NYYMS

SEQ ID NO: 66 - protein, 17 aa, synthetic construct
  REGION 1..17: note = Girentuximab humanised VH1 Variable Heavy chain - HCDR2
AINSDGGITYYLDTVKG

SEQ ID NO: 67 - protein, 10 aa, synthetic construct
  REGION 1..10: note = Girentuximab humanised VH1 Variable Heavy chain - HCDR3 (protein)
HRSGYFSMDY

SEQ ID NO: 68 - protein, 119 aa, synthetic construct
  REGION 1..119: note = Girentuximab humanised VH1 Variable Heavy chain - VH (protein)
DVKLVESGGGLVKPGGSLRLSCAASGFTFSNYYMSWVRQAPGKGLELVAAINSDGGITYYLDTVKGRFTISRDNAKNTLYLQMSSLKSEDTALYYCARHRSGYFSMDYWGQGTSVTVSS

SEQ ID NO: 69 - DNA, 15 bp, synthetic construct
  misc_feature 1..15: note = Girentuximab humanised VH1 Variable Heavy chain - HCDR1 (DNA)
  source 1..15: mol_type = other DNA
aactactacatgagc

SEQ ID NO: 70 - DNA, 51 bp, synthetic construct
  misc_feature 1..51: note = Girentuximab humanised VH1 Variable Heavy chain - HCDR2 (DNA)
  source 1..51: mol_type = other DNA
gccattaacagtgacggtggcatcacctactacctggacaccgtgaagggc

SEQ ID NO: 71 - DNA, 30 bp, synthetic construct
  misc_feature 1..30: note = Girentuximab humanised VH1 Variable Heavy chain - HCDR3 (DNA)
  source 1..30: mol_type = other DNA
cacaggagcggctacttctctatggactac

SEQ ID NO: 72 - DNA, 357 bp, synthetic construct
  misc_feature 1..357: note = Girentuximab humanised VH1 Variable Heavy chain - VH (DNA)
  source 1..357: mol_type = other DNA
gacgtgaagctggtggagtctgggggaggcttggtcaagcctggaggatccctgagactctcctgtgcagcctctggattcaccttcagtaactactacatgagctgggtgcgccaggctccagggaaggggctggagctggttgccgccattaacagtgacggtggcatcacctactacctggacaccgtgaagggccgattcaccatctccagggacaacgccaagaacaccctgtatctgcaaatgagcagcctgaagagcgaggacacggccctgtattactgtgcgagacacaggagcggctacttctctatggactactggggccagggcaccagcgtcactgtctcctca

SEQ ID NO: 73 - protein, 30 aa, synthetic construct
  REGION 1..30: note = Girentuximab humanised VH1 Variable Heavy chain - HFR1 (protein)
DVKLVESGGGLVKPGGSLRLSCAASGFTFS

SEQ ID NO: 74 - protein, 14 aa, synthetic construct
  REGION 1..14: note = Girentuximab humanised VH1 Variable Heavy chain - HFR2 (protein)
WVRQAPGKGLELVA

SEQ ID NO: 75 - protein, 32 aa, synthetic construct
  REGION 1..32: note = Girentuximab humanised VH1 Variable Heavy chain - HFR3 (protein)
RFTISRDNAKNTLYLQMSSLKSEDTALYYCAR

SEQ ID NO: 76 - protein, 11 aa, synthetic construct
  REGION 1..11: note = Girentuximab humanised VH1 Variable Heavy chain - HFR4 (protein)
WGQGTSVTVSS

SEQ ID NO: 77 - DNA, 90 bp, synthetic construct
  misc_feature 1..90: note = Girentuximab humanised VH1 Variable Heavy chain - HFR1 (DNA)
  source 1..90: mol_type = other DNA
gacgtgaagctggtggagtctgggggaggcttggtcaagcctggaggatccctgagactctcctgtgcagcctctggattcaccttcagt

SEQ ID NO: 78 - DNA, 42 bp, synthetic construct
  misc_feature 1..42: note = Girentuximab humanised VH1 Variable Heavy chain - HFR2 (DNA)
  source 1..42: mol_type = other DNA
tgggtgcgccaggctccagggaaggggctggagctggttgcc

SEQ ID NO: 79 - DNA, 96 bp, synthetic construct
  misc_feature 1..96: note = Girentuximab humanised VH1 Variable Heavy chain - HFR3 (DNA)
  source 1..96: mol_type = other DNA
cgattcaccatctccagggacaacgccaagaacaccctgtatctgcaaatgagcagcctgaagagcgaggacacggccctgtattactgtgcgaga

SEQ ID NO: 80 - DNA, 33 bp, synthetic construct
  misc_feature 1..33: note = Girentuximab humanised VH1 Variable Heavy chain - HFR4 (DNA)
  source 1..33: mol_type = other DNA
tggggccagggcaccagcgtcactgtctcctca

SEQ ID NO: 81 - protein, 5 aa, synthetic construct
  REGION 1..5: note = Girentuximab humanised VH3 Variable Heavy chain - HCDR1 (protein)
NYYMS

SEQ ID NO: 82 - protein, 17 aa, synthetic construct
  REGION 1..17: note = Girentuximab humanised VH3 Variable Heavy chain - HCDR2 (protein)
AINSDGGITYYLDTVKG

SEQ ID NO: 83 - protein, 10 aa, synthetic construct
  REGION 1..10: note = Girentuximab humanised VH3 Variable Heavy chain - HCDR3 (protein)
HRSGYFSMDY

SEQ ID NO: 84 - protein, 119 aa, synthetic construct
  REGION 1..119: note = Girentuximab humanised VH3 Variable Heavy chain - VH (protein)
DVKLVESGGGLVKPGGSLRLSCAASGFTFSNYYMSWVRQAPGKGLELVAAINSDGGITYYLDTVKGRFTISRDNAKNTLYLQMNSLRAEDTALYYCARHRSGYFSMDYWGQGTLVTVSS

SEQ ID NO: 85 - DNA, 15 bp, synthetic construct
  misc_feature 1..15: note = Girentuximab humanised VH3 Variable Heavy chain - HCDR1 (DNA)
  source 1..15: mol_type = other DNA
aactactacatgagc

SEQ ID NO: 86 - DNA, 50 bp, synthetic construct
  misc_feature 1..50: note = Girentuximab humanised VH3 Variable Heavy chain - HCDR2 (DNA)
  source 1..50: mol_type = other DNA
ccattaacagtgacggtggcatcacctactacctggacaccgtgaagggc

SEQ ID NO: 87 - DNA, 30 bp, synthetic construct; identical to SEQ ID NO: 71
  misc_feature 1..30: note = Girentuximab humanised VH3 Variable Heavy chain - HCDR3 (DNA)
  source 1..30: mol_type = other DNA

SEQ ID NO: 88 - DNA, 357 bp, synthetic construct
  misc_feature 1..357: note = Girentuximab humanised VH3 Variable Heavy chain - VH (DNA)
  source 1..357: mol_type = other DNA
gacgtgaagctggtggagtctgggggaggcttggtcaagcctggaggatccctgagactctcctgtgcagcctctggattcaccttcagtaactactacatgagctgggtgcgccaggctccagggaaggggctggagctggttgccgccattaacagtgacggtggcatcacctactacctggacaccgtgaagggccgattcaccatctccagggacaacgccaagaacaccctgtatctgcaaatgaacagcctgagggccgaggacacggccctgtattactgtgcgagacacaggagcggctacttctctatggactactggggccagggcaccctggtcactgtctcctca

SEQ ID NO: 89 - protein, 30 aa, synthetic construct; identical to SEQ ID NO: 73
  REGION 1..30: note = Girentuximab humanised VH3 Variable Heavy chain - HFR1 (protein)

SEQ ID NO: 90 - protein, 14 aa, synthetic construct
  REGION 1..14: note = Girentuximab humanised VH3 Variable Heavy chain - HFR2 (protein)
WVRQAPGKGLELVA

SEQ ID NO: 91 - protein, 32 aa, synthetic construct
  REGION 1..32: note = Girentuximab humanised VH3 Variable Heavy chain - HFR3 (protein)
RFTISRDNAKNTLYLQMNSLRAEDTALYYCAR

SEQ ID NO: 92 - protein, 11 aa, synthetic construct
  REGION 1..11: note = Girentuximab humanised VH3 Variable Heavy chain - HFR4 (protein)
WGQGTLVTVSS

SEQ ID NO: 93 - DNA, 90 bp, synthetic construct; identical to SEQ ID NO: 77
  misc_feature 1..90: note = Girentuximab humanised VH3 Variable Heavy chain - HFR1 (DNA)
  source 1..90: mol_type = other DNA

SEQ ID NO: 94 - DNA, 42 bp, synthetic construct; identical to SEQ ID NO: 78
  misc_feature 1..42: note = Girentuximab humanised VH3 Variable Heavy chain - HFR2 (DNA)
  source 1..42: mol_type = other DNA

SEQ ID NO: 95 - DNA, 96 bp, synthetic construct
  misc_feature 1..96: note = Girentuximab humanised VH3 Variable Heavy chain - HFR3 (DNA)
  source 1..96: mol_type = other DNA
cgattcaccatctccagggacaacgccaagaacaccctgtatctgcaaatgaacagcctgagggccgaggacacggccctgtattactgtgcgaga

SEQ ID NO: 96 - DNA, 33 bp, synthetic construct
  misc_feature 1..33: note = Girentuximab humanised VH3 Variable Heavy chain - HFR4 (DNA)
  source 1..33: mol_type = other DNA
tggggccagggcaccctggtcactgtctcctca

SEQ ID NO: 97 - protein, 5 aa, synthetic construct
  REGION 1..5: note = Girentuximab humanised VH4 Variable Heavy chain - HCDR1 (protein)
NYYMS

SEQ ID NO: 98 - protein, 17 aa, synthetic construct
  REGION 1..17: note = Girentuximab humanised VH4 Variable Heavy chain - HCDR2 (protein)
AINSDGGITYYLDTVKG

SEQ ID NO: 99 - protein, 10 aa, synthetic construct
  REGION 1..10: note = Girentuximab humanised VH4 Variable Heavy chain - HCDR3 (protein)
HRSGYFSMDY

SEQ ID NO: 100 - protein, 119 aa, synthetic construct
  REGION 1..119: note = Girentuximab humanised VH4 Variable Heavy chain - VH (protein)
EVQLVESGGGLVKPGGSLRLSCAASGFTFSNYYMSWVRQAPGKGLELVAAINSDGGITYYLDTVKGRFTISRDNAKNTLYLQMNSLRAEDTALYYCARHRSGYFSMDYWGQGTLVTVSS

SEQ ID NO: 101 - DNA, 15 bp, synthetic construct
  misc_feature 1..15: note = Girentuximab humanised VH4 Variable Heavy chain - HCDR1 (DNA)
  source 1..15: mol_type = other DNA
aactactacatgagc

SEQ ID NO: 102 - DNA, 51 bp, synthetic construct; identical to SEQ ID NO: 70
  misc_feature 1..51: note = Girentuximab humanised VH4 Variable Heavy chain - HCDR2 (DNA)
  source 1..51: mol_type = other DNA

SEQ ID NO: 103 - DNA, 30 bp, synthetic construct; identical to SEQ ID NO: 71
  misc_feature 1..30: note = Girentuximab humanised VH4 Variable Heavy chain - HCDR3 (DNA)
  source 1..30: mol_type = other DNA

SEQ ID NO: 104 - DNA, 357 bp, synthetic construct
  misc_feature 1..357: note = Girentuximab humanised VH4 Variable Heavy chain - VH (DNA)
  source 1..357: mol_type = other DNA
gaggtgcagctggtggagtctgggggaggcttggtcaagcctggaggatccctgagactctcctgtgcagcctctggattcaccttcagtaactactacatgagctgggtgcgccaggctccagggaaggggctggagctggttgccgccattaacagtgacggtggcatcacctactacctggacaccgtgaagggccgattcaccatctccagggacaacgccaagaacaccctgtatctgcaaatgaacagcctgagggccgaggacacggccctgtattactgtgcgagacacaggagcggctacttctctatggactactggggccagggcaccctggtcactgtctcctca

SEQ ID NO: 105 - protein, 30 aa, synthetic construct
  REGION 1..30: note = Girentuximab humanised VH4 Variable Heavy chain - HFR1 (protein)
EVQLVESGGGLVKPGGSLRLSCAASGFTFS

SEQ ID NO: 106 - protein, 14 aa, synthetic construct
  REGION 1..14: note = Girentuximab humanised VH4 Variable Heavy chain - HFR2 (protein)
WVRQAPGKGLELVA

SEQ ID NO: 107 - protein, 32 aa, synthetic construct; identical to SEQ ID NO: 91
  REGION 1..32: note = Girentuximab humanised VH4 Variable Heavy chain - HFR3 (protein)

SEQ ID NO: 108 - protein, 11 aa, synthetic construct
  REGION 1..11: note = Girentuximab humanised VH4 Variable Heavy chain - HFR4 (protein)
WGQGTLVTVSS

SEQ ID NO: 109 - DNA, 90 bp, synthetic construct
  misc_feature 1..90: note = Girentuximab humanised VH4 Variable Heavy chain - HFR1 (DNA)
  source 1..90: mol_type = other DNA
gaggtgcagctggtggagtctgggggaggcttggtcaagcctggaggatccctgagactctcctgtgcagcctctggattcaccttcagt

SEQ ID NO: 110 - DNA, 42 bp, synthetic construct; identical to SEQ ID NO: 78
  misc_feature 1..42: note = Girentuximab humanised VH4 Variable Heavy chain - HFR2 (DNA)
  source 1..42: mol_type = other DNA

SEQ ID NO: 111 - DNA, 96 bp, synthetic construct; identical to SEQ ID NO: 95
  misc_feature 1..96: note = Girentuximab humanised VH4 Variable Heavy chain - HFR3 (DNA)
  source 1..96: mol_type = other DNA

SEQ ID NO: 112 - DNA, 33 bp, synthetic construct; identical to SEQ ID NO: 96
  misc_feature 1..33: note = Girentuximab humanised VH4 Variable Heavy chain - HFR4 (DNA)
  source 1..33: mol_type = other DNA

SEQ ID NO: 113 - protein, 5 aa, synthetic construct
  REGION 1..5: note = Girentuximab humanised VH5 Variable Heavy chain - HCDR1 (protein)
NYYMS

SEQ ID NO: 114 - protein, 17 aa, synthetic construct
  REGION 1..17: note = Girentuximab humanised VH5 Variable Heavy chain - HCDR2 (protein)
AINSDGGITYYLDTVKG

SEQ ID NO: 115 - protein, 10 aa, synthetic construct
  REGION 1..10: note = Girentuximab humanised VH5 Variable Heavy chain - HCDR3 (protein)
HRSGYFSMDY

SEQ ID NO: 116 - protein, 119 aa, synthetic construct
  REGION 1..119: note = Girentuximab humanised VH5 Variable Heavy chain - VH (protein)
EVQLVESGGGLVKPGGSLRLSCAASGFTFSNYYMSWVRQAPGKGLEWVAAINSDGGITYYLDTVKGRFTISRDNAKNTLYLQMNSLRAEDTALYYCARHRSGYFSMDYWGQGTLVTVSS

SEQ ID NO: 117 - DNA, 15 bp, synthetic construct
  misc_feature 1..15: note = Girentuximab humanised VH5 Variable Heavy chain - HCDR1 (DNA)
  source 1..15: mol_type = other DNA
aactactacatgagc

SEQ ID NO: 118 - DNA, 51 bp, synthetic construct; identical to SEQ ID NO: 70
  misc_feature 1..51: note = Girentuximab humanised VH5 Variable Heavy chain - HCDR2 (DNA)
  source 1..51: mol_type = other DNA

SEQ ID NO: 119 - DNA, 30 bp, synthetic construct; identical to SEQ ID NO: 71
  misc_feature 1..30: note = Girentuximab humanised VH5 Variable Heavy chain - HCDR3 (DNA)
  source 1..30: mol_type = other DNA

SEQ ID NO: 120 - DNA, 357 bp, synthetic construct
  misc_feature 1..357: note = Girentuximab humanised VH5 Variable Heavy chain - VH (DNA)
  source 1..357: mol_type = other DNA
gaggtgcagctggtggagtctgggggaggcttggtcaagcctggaggatccctgagactctcctgtgcagcctctggattcaccttcagtaactactacatgagctgggtgcgccaggctccagggaaggggctggagtgggttgccgccattaacagtgacggtggcatcacctactacctggacaccgtgaagggccgattcaccatctccagggacaacgccaagaacaccctgtatctgcaaatgaacagcctgagggccgaggacacggccctgtattactgtgcgagacacaggagcggctacttctctatggactactggggccagggcaccctggtcactgtctcctca

SEQ ID NO: 121 - protein, 30 aa, synthetic construct; identical to SEQ ID NO: 105
  REGION 1..30: note = Girentuximab humanised VH5 Variable Heavy chain - HFR1 (protein)

SEQ ID NO: 122 - protein, 14 aa, synthetic construct
  REGION 1..14: note = Girentuximab humanised VH5 Variable Heavy chain - HFR2 (protein)
WVRQAPGKGLEWVA

SEQ ID NO: 123 - protein, 32 aa, synthetic construct; identical to SEQ ID NO: 91
  REGION 1..32: note = Girentuximab humanised VH5 Variable Heavy chain - HFR3 (protein)

SEQ ID NO: 124 - protein, 11 aa, synthetic construct
  REGION 1..11: note = Girentuximab humanised VH5 Variable Heavy chain - HFR4 (protein)
WGQGTLVTVSS

SEQ ID NO: 125 - DNA, 90 bp, synthetic construct; identical to SEQ ID NO: 109
  misc_feature 1..90: note = Girentuximab humanised VH5 Variable Heavy chain - HFR1 (DNA)
  source 1..90: mol_type = other DNA

SEQ ID NO: 126 - DNA, 42 bp, synthetic construct
  misc_feature 1..42: note = Girentuximab humanised VH5 Variable Heavy chain - HFR2 (DNA)
  source 1..42: mol_type = other DNA
tgggtgcgccaggctccagggaaggggctggagtgggttgcc

SEQ ID NO: 127 - DNA, 96 bp, synthetic construct; identical to SEQ ID NO: 95
  misc_feature 1..96: note = Girentuximab humanised VH5 Variable Heavy chain - HFR3 (DNA)
  source 1..96: mol_type = other DNA

SEQ ID NO: 128 - DNA, 33 bp, synthetic construct; identical to SEQ ID NO: 96
  misc_feature 1..33: note = Girentuximab humanised VH5 Variable Heavy chain - HFR4 (DNA)
  source 1..33: mol_type = other DNA

SEQ ID NO: 129 - protein, 11 aa, synthetic construct
  REGION 1..11: note = Girentuximab Chimeric Vk0 Variable light chain- LCDR1 (protein)
KASQNVVSAVA

SEQ ID NO: 130 - protein, 7 aa, synthetic construct
  REGION 1..7: note = Girentuximab Chimeric Vk0 Variable light chain- LCDR2 (protein)
SASNRYT

SEQ ID NO: 131 - protein, 9 aa, synthetic construct
  REGION 1..9: note = Girentuximab Chimeric Vk0 Variable light chain- LCDR3 (protein)
QQYSNYPWT

SEQ ID NO: 132 - protein, 107 aa, synthetic construct
  REGION 1..107: note = Girentuximab Chimeric Vk0 Variable light chain- VL (protein)
DIVMTQSQRFMSTTVGDRVSITCKASQNVVSAVAWYQQKPGQSPKLLIYSASNRYTGVPDRFTGSGSGTDFTLTISNMQSEDLADFFCQQYSNYPWTFGGGTKLEIK

SEQ ID NO: 133 - DNA, 33 bp, synthetic construct
  misc_feature 1..33: note = Girentuximab Chimeric Vk0 Variable light chain- LCDR1 (DNA)
  source 1..33: mol_type = other DNA
aaggccagtcagaatgtggtttctgctgttgcc

SEQ ID NO: 134 - DNA, 21 bp, synthetic construct
  misc_feature 1..21: note = Girentuximab Chimeric Vk0 Variable light chain- LCDR2 (DNA)
  source 1..21: mol_type = other DNA
tcagcatccaatcggtacact

SEQ ID NO: 135 - DNA, 27 bp, synthetic construct
  misc_feature 1..27: note = Girentuximab Chimeric Vk0 Variable light chain- LCDR3 (DNA)
  source 1..27: mol_type = other DNA
caacaatatagcaactatccgtggacg

SEQ ID NO: 136 - DNA, 321 bp, synthetic construct
  misc_feature 1..321: note = Girentuximab Chimeric Vk0 Variable light chain- VL (DNA)
  source 1..321: mol_type = other DNA
gacattgtgatgacccagtctcaaagattcatgtccacaacagtaggagacagggtcagcatcacctgcaaggccagtcagaatgtggtttctgctgttgcctggtatcaacagaaaccaggacaatctcctaaactactgatttactcagcatccaatcggtacactggagtccctgatcgcttcacaggcagtggatctgggacagatttcactctcaccattagcaatatgcagtctgaagacctggctgattttttctgtcaacaatatagcaactatccgtggacgttcggtggaggcaccaagctggaaatcaaa

SEQ ID NO: 137 - protein, 23 aa, synthetic construct
  REGION 1..23: note = Girentuximab Chimeric Vk0 Variable light chain- LFR1 (protein)
DIVMTQSQRFMSTTVGDRVSITC

SEQ ID NO: 138 - protein, 15 aa, synthetic construct
  REGION 1..15: note = Girentuximab Chimeric Vk0 Variable light chain- LFR2 (protein)
WYQQKPGQSPKLLIY

SEQ ID NO: 139 - protein, 32 aa, synthetic construct
  REGION 1..32: note = Girentuximab Chimeric Vk0 Variable light chain- LFR3 (protein)
GVPDRFTGSGSGTDFTLTISNMQSEDLADFFC

SEQ ID NO: 140 - protein, 10 aa, synthetic construct
  REGION 1..10: note = Girentuximab Chimeric Vk0 Variable light chain- LFR4 (protein)
FGGGTKLEIK

SEQ ID NO: 141 - DNA, 69 bp, synthetic construct
  misc_feature 1..69: note = Girentuximab Chimeric Vk0 Variable light chain- LFR1 (DNA)
  source 1..69: mol_type = other DNA
gacattgtgatgacccagtctcaaagattcatgtccacaacagtaggagacagggtcagcatcacctgc

SEQ ID NO: 142 - DNA, 45 bp, synthetic construct
  misc_feature 1..45: note = Girentuximab Chimeric Vk0 Variable light chain- LFR2 (DNA)
  source 1..45: mol_type = other DNA
tggtatcaacagaaaccaggacaatctcctaaactactgatttac

SEQ ID NO: 143 - DNA, 96 bp, synthetic construct
  misc_feature 1..96: note = Girentuximab Chimeric Vk0 Variable light chain- LFR3 (DNA)
  source 1..96: mol_type = other DNA
ggagtccctgatcgcttcacaggcagtggatctgggacagatttcactctcaccattagcaatatgcagtctgaagacctggctgattttttctgt

SEQ ID NO: 144 - DNA, 30 bp, synthetic construct
  misc_feature 1..30: note = Girentuximab Chimeric Vk0 Variable light chain- LFR4 (DNA)
  source 1..30: mol_type = other DNA
ttcggtggaggcaccaagctggaaatcaaa

SEQ ID NO: 145 - protein, 11 aa, synthetic construct
  REGION 1..11: note = Girentuximab Humanised Vk1 Variable light chain- LCDR1 (protein)
KASQNVVSAVA

SEQ ID NO: 146 - protein, 7 aa, synthetic construct
  REGION 1..7: note = Girentuximab Humanised Vk1 Variable light chain- LCDR2 (protein)
SASNRYT

SEQ ID NO: 147 - protein, 9 aa, synthetic construct
  REGION 1..9: note = Girentuximab Humanised Vk1 Variable light chain- LCDR3 (protein)
QQYSNYPWT

SEQ ID NO: 148 - protein, 107 aa, synthetic construct
  REGION 1..107: note = Girentuximab Humanised Vk1 Variable light chain- VL (protein)
DIVMTQSPSFLSASVGDRVTITCKASQNVVSAVAWYQQKPGQAPKLLIYSASNRYTGVPDRFTGSGSGTDFTLTISSLQSEDLADYFCQQYSNYPWTFGGGTKVEIK

SEQ ID NO: 149 - DNA, 33 bp, synthetic construct
  misc_feature 1..33: note = Girentuximab Humanised Vk1 Variable light chain- LCDR1 (DNA)
  source 1..33: mol_type = other DNA
aaggcaagtcagaacgtggtgagtgctgtggcc

SEQ ID NO: 150 - DNA, 21 bp, synthetic construct
  misc_feature 1..21: note = Girentuximab Humanised Vk1 Variable light chain- LCDR2 (DNA)
  source 1..21: mol_type = other DNA
agcgcctccaacaggtacacc

SEQ ID NO: 151 - DNA, 27 bp, synthetic construct
  misc_feature 1..27: note = Girentuximab Humanised Vk1 Variable light chain- LCDR3 (DNA)
  source 1..27: mol_type = other DNA
caacagtacagcaattacccttggacg

SEQ ID NO: 152 - DNA, 321 bp, synthetic construct
  misc_feature 1..321: note = Girentuximab Humanised Vk1 Variable light chain- VL (DNA)
  source 1..321: mol_type = other DNA
gacatcgtgatgacccagtctccatccttcctgtctgcatctgtaggagacagagtcaccatcacttgcaaggcaagtcagaacgtggtgagtgctgtggcctggtatcagcagaaaccagggcaggctcctaagctcctgatctatagcgcctccaacaggtacaccggggtcccagacaggttcaccggcagtggatctgggacagatttcactctcaccatcagcagcctgcagagcgaagatctggcagactatttctgtcaacagtacagcaattacccttggacgttcggcggcgggaccaaggtggaaatcaaa

SEQ ID NO: 153 - protein, 23 aa, synthetic construct
  REGION 1..23: note = Girentuximab Humanised Vk1 Variable light chain- LFR1 (protein)
DIVMTQSPSFLSASVGDRVTITC

SEQ ID NO: 154 - protein, 15 aa, synthetic construct
  REGION 1..15: note = Girentuximab Humanised Vk1 Variable light chain- LFR2 (protein)
WYQQKPGQAPKLLIY

SEQ ID NO: 155 - protein, 32 aa, synthetic construct
  REGION 1..32: note = Girentuximab Humanised Vk1 Variable light chain- LFR3 (protein)
GVPDRFTGSGSGTDFTLTISSLQSEDLADYFC

SEQ ID NO: 156 - protein, 10 aa, synthetic construct
  REGION 1..10: note = Girentuximab Humanised Vk1 Variable light chain- LFR4 (protein)
FGGGTKVEIK

SEQ ID NO: 157 - DNA, 69 bp, synthetic construct
  misc_feature 1..69: note = Girentuximab Humanised Vk1 Variable light chain- LFR1 (DNA)
  source 1..69: mol_type = other DNA
gacatcgtgatgacccagtctccatccttcctgtctgcatctgtaggagacagagtcaccatcacttgc

SEQ ID NO: 158 - DNA, 45 bp, synthetic construct
  misc_feature 1..45: note = Girentuximab Humanised Vk1 Variable light chain- LFR2 (DNA)
  source 1..45: mol_type = other DNA
tggtatcagcagaaaccagggcaggctcctaagctcctgatctat

SEQ ID NO: 159 - DNA, 96 bp, synthetic construct
  misc_feature 1..96: note = Girentuximab Humanised Vk1 Variable light chain- LFR3 (DNA)
  source 1..96: mol_type = other DNA
ggggtcccagacaggttcaccggcagtggatctgggacagatttcactctcaccatcagcagcctgcagagcgaagatctggcagactatttctgt

SEQ ID NO: 160 - DNA, 30 bp, synthetic construct
  misc_feature 1..30: note = Girentuximab Humanised Vk1 Variable light chain- LFR4 (DNA)
  source 1..30: mol_type = other DNA
ttcggcggcgggaccaaggtggaaatcaaa

SEQ ID NO: 161 - protein, 11 aa, synthetic construct
  REGION 1..11: note = Girentuximab Humanised Vk2 Variable light chain- LCDR1 (protein)
KASQNVVSAVA

SEQ ID NO: 162 - protein, 7 aa, synthetic construct
  REGION 1..7: note = Girentuximab Humanised Vk2 Variable light chain- LCDR2 (protein)
SASNRYT

SEQ ID NO: 163 - protein, 9 aa, synthetic construct
  REGION 1..9: note = Girentuximab Humanised Vk2 Variable light chain- LCDR3 (protein)
QQYSNYPWT

SEQ ID NO: 164 - protein, 107 aa, synthetic construct
  REGION 1..107: note = Girentuximab Humanised Vk2 Variable light chain- VL (protein)
DIVMTQSPSSLSASVGDRVTITCKASQNVVSAVAWYQQKPGQAPRLLIYSASNRYTGVPDRFTGSGSGTDFTLTISSLQAEDLADYFCQQYSNYPWTFGGGTKVEIK

SEQ ID NO: 165 - DNA, 33 bp, synthetic construct; identical to SEQ ID NO: 149
  misc_feature 1..33: note = Girentuximab Humanised Vk2 Variable light chain- LCDR1 (DNA)
  source 1..33: mol_type = other DNA

SEQ ID NO: 166 - DNA, 21 bp, synthetic construct
  misc_feature 1..21: note = Girentuximab Humanised Vk2 Variable light chain- LCDR2 (DNA)
  source 1..21: mol_type = other DNA
agcgcctccaacaggtacacc

SEQ ID NO: 167 - DNA, 27 bp, synthetic construct
  misc_feature 1..27: note = Girentuximab Humanised Vk2 Variable light chain- LCDR3 (DNA)
  source 1..27: mol_type = other DNA
caacagtacagcaattacccttggacg

SEQ ID NO: 168 - DNA, 321 bp, synthetic construct
  misc_feature 1..321: note = Girentuximab Humanised Vk2 Variable light chain- VL (DNA)
  source 1..321: mol_type = other DNA
gacatcgtgatgacccagtctccatccagcctgtctgcatctgtaggagacagagtcaccatcacttgcaaggcaagtcagaacgtggtgagtgctgtggcctggtatcagcagaaaccagggcaggctcctaggctcctgatctatagcgcctccaacaggtacaccggggtcccagacaggttcaccggcagtggatctgggacagatttcactctcaccatcagcagcctgcaggccgaagatctggcagactatttctgtcaacagtacagcaattacccttggacgttcggcggcgggaccaaggtggaaatcaaa

SEQ ID NO: 169 - protein, 23 aa, synthetic construct
  REGION 1..23: note = Girentuximab Humanised Vk2 Variable light chain- LFR1 (protein)
DIVMTQSPSSLSASVGDRVTITC

SEQ ID NO: 170 - protein, 15 aa, synthetic construct
  REGION 1..15: note = Girentuximab Humanised Vk2 Variable light chain- LFR2 (protein)
WYQQKPGQAPRLLIY

SEQ ID NO: 171 - protein, 32 aa, synthetic construct
  REGION 1..32: note = Girentuximab Humanised Vk2 Variable light chain- LFR3 (protein)
GVPDRFTGSGSGTDFTLTISSLQAEDLADYFC

SEQ ID NO: 172 - protein, 10 aa, synthetic construct
  REGION 1..10: note = Girentuximab Humanised Vk2 Variable light chain- LFR4 (protein)
FGGGTKVEIK

SEQ ID NO: 173 - DNA, 69 bp, synthetic construct
  misc_feature 1..69: note = Girentuximab Humanised Vk2 Variable light chain- LFR1 (DNA)
  source 1..69: mol_type = other DNA
gacatcgtgatgacccagtctccatccagcctgtctgcatctgtaggagacagagtcaccatcacttgc

SEQ ID NO: 174 - DNA, 45 bp, synthetic construct
  misc_feature 1..45: note = Girentuximab Humanised Vk2 Variable light chain- LFR2 (DNA)
  source 1..45: mol_type = other DNA
tggtatcagcagaaaccagggcaggctcctaggctcctgatctat

SEQ ID NO: 175 - DNA, 96 bp, synthetic construct
  misc_feature 1..96: note = Girentuximab Humanised Vk2 Variable light chain- LFR3 (DNA)
  source 1..96: mol_type = other DNA
ggggtcccagacaggttcaccggcagtggatctgggacagatttcactctcaccatcagcagcctgcaggccgaagatctggcagactatttctgt

SEQ ID NO: 176 - DNA, 30 bp, synthetic construct; identical to SEQ ID NO: 160
  misc_feature 1..30: note = Girentuximab Humanised Vk2 Variable light chain- LFR4 (DNA)
  source 1..30: mol_type = other DNA

SEQ ID NO: 177 - protein, 11 aa, synthetic construct
  REGION 1..11: note = Girentuximab Humanised Vk3 Variable light chain- LCDR1 (protein)
KASQNVVSAVA

SEQ ID NO: 178 - protein, 7 aa, synthetic construct
  REGION 1..7: note = Girentuximab Humanised Vk3 Variable light chain- LCDR2 (protein)
SASNRYT

SEQ ID NO: 179 - protein, 9 aa, synthetic construct
  REGION 1..9: note = Girentuximab Humanised Vk3 Variable light chain- LCDR3 (protein)
QQYSNYPWT

SEQ ID NO: 180 - protein, 107 aa, synthetic construct
  REGION 1..107: note = Girentuximab Humanised Vk3 Variable light chain- VL (protein)
DIQMTQSPSSLSASVGDRVTITCKASQNVVSAVAWYQQKPGQAPRLLIYSASNRYTGVPDRFSGSGSGTDFTLTISSLQAEDLADYFCQQYSNYPWTFGGGTKVEIK

SEQ ID NO: 181 - DNA, 33 bp, synthetic construct; identical to SEQ ID NO: 149
  misc_feature 1..33: note = Girentuximab Humanised Vk3 Variable light chain- LCDR1 (DNA)
  source 1..33: mol_type = other DNA

SEQ ID NO: 182 - DNA, 21 bp, synthetic construct
  misc_feature 1..21: note = Girentuximab Humanised Vk3 Variable light chain- LCDR2 (DNA)
  source 1..21: mol_type = other DNA
agcgcctccaacaggtacacc

SEQ ID NO: 183 - DNA, 27 bp, synthetic construct
  misc_feature 1..27: note = Girentuximab Humanised Vk3 Variable light chain- LCDR3 (DNA)
  source 1..27: mol_type = other DNA
caacagtacagcaattacccttggacg

SEQ ID NO: 184 - DNA, 321 bp, synthetic construct
  misc_feature 1..321: note = Girentuximab Humanised Vk3 Variable light chain- VL (DNA)
  source 1..321: mol_type = other DNA
gacatccagatgacccagtctccatccagcctgtctgcatctgtaggagacagagtcaccatcacttgcaaggcaagtcagaacgtggtgagtgctgtggcctggtatcagcagaaaccagggcaggctcctaggctcctgatctatagcgcctccaacaggtacaccggggtcccagacaggttcagcggcagtggatctgggacagatttcactctcaccatcagcagcctgcaggccgaagatctggcagactatttctgtcaacagtacagcaattacccttggacgttcggcggcgggaccaaggtggaaatcaaa

SEQ ID NO: 185 - protein, 23 aa, synthetic construct
  REGION 1..23: note = Girentuximab Humanised Vk3 Variable light chain- LFR1 (protein)
DIQMTQSPSSLSASVGDRVTITC

SEQ ID NO: 186 - protein, 15 aa, synthetic construct
  REGION 1..15: note = Girentuximab Humanised Vk3 Variable light chain- LFR2 (protein)
WYQQKPGQAPRLLIY

SEQ ID NO: 187 - protein, 32 aa, synthetic construct
  REGION 1..32: note = Girentuximab Humanised Vk3 Variable light chain- LFR3 (protein)
GVPDRFSGSGSGTDFTLTISSLQAEDLADYFC

SEQ ID NO: 188 - protein, 10 aa, synthetic construct
  REGION 1..10: note = Girentuximab Humanised Vk3 Variable light chain- LFR4 (protein)
FGGGTKVEIK

SEQ ID NO: 189 - DNA, 69 bp, synthetic construct
  misc_feature 1..69: note = Girentuximab Humanised Vk3 Variable light chain- LFR1 (DNA)
  source 1..69: mol_type = other DNA
gacatccagatgacccagtctccatccagcctgtctgcatctgtaggagacagagtcaccatcacttgc

SEQ ID NO: 190 - DNA, 45 bp, synthetic construct; identical to SEQ ID NO: 174
  misc_feature 1..45: note = Girentuximab Humanised Vk3 Variable light chain- LFR2 (DNA)
  source 1..45: mol_type = other DNA

SEQ ID NO: 191 - DNA, 96 bp, synthetic construct
  misc_feature 1..96: note = Girentuximab Humanised Vk3 Variable light chain- LFR3 (DNA)
  source 1..96: mol_type = other DNA
ggggtcccagacaggttcagcggcagtggatctgggacagatttcactctcaccatcagcagcctgcaggccgaagatctggcagactatttctgt

SEQ ID NO: 192 - DNA, 30 bp, synthetic construct; identical to SEQ ID NO: 160
  misc_feature 1..30: note = Girentuximab Humanised Vk3 Variable light chain- LFR4 (DNA)
  source 1..30: mol_type = other DNA

SEQ ID NO: 193 - protein, 11 aa, synthetic construct
  REGION 1..11: note = Girentuximab Humanised Vk4 Variable light chain- LCDR1 (protein)
KASQNVVSAVA

SEQ ID NO: 194 - protein, 7 aa, synthetic construct
  REGION 1..7: note = Girentuximab Humanised Vk4 Variable light chain- LCDR2 (protein)
SASNRYT

SEQ ID NO: 195 - protein, 9 aa, synthetic construct
  REGION 1..9: note = Girentuximab Humanised Vk4 Variable light chain- LCDR3 (protein)
QQYSNYPWT

SEQ ID NO: 196 - protein, 107 aa, synthetic construct
  REGION 1..107: note = Girentuximab Humanised Vk4 Variable light chain- VL (protein)
DIQMTQSPSSLSASVGDRVTITCKASQNVVSAVAWYQQKPGQAPRLLIYSASNRYTGVPDRFSGSGSGTDFTLTISSLQAEDLADYYCQQYSNYPWTFGGGTKVEIK

SEQ ID NO: 197 - DNA, 33 bp, synthetic construct; identical to SEQ ID NO: 149
  misc_feature 1..33: note = Girentuximab Humanised Vk4 Variable light chain- LCDR1 (DNA)
  source 1..33: mol_type = other DNA

SEQ ID NO: 198 - DNA, 21 bp, synthetic construct
  misc_feature 1..21: note = Girentuximab Humanised Vk4 Variable light chain- LCDR2 (DNA)
  source 1..21: mol_type = other DNA
agcgcctccaacaggtacacc

SEQ ID NO: 199 - DNA, 27 bp, synthetic construct
  misc_feature 1..27: note = Girentuximab Humanised Vk4 Variable light chain- LCDR3 (DNA)
  source 1..27: mol_type = other DNA
caacagtacagcaattacccttggacg

SEQ ID NO: 200 - DNA, 321 bp, synthetic construct
  misc_feature 1..321: note = Girentuximab Humanised Vk4 Variable light chain - VL (DNA)
  source 1..321: mol_type = other DNA
gacatccagatgacccagtctccatccagcctgtctgcatctgtaggagacagagtcaccatcacttgcaaggcaagtcagaacgtggtgagtgctgtggcctggtatcagcagaaaccagggcaggctcctaggctcctgatctatagcgcctccaacaggtacaccggggtcccagacaggttcagcggcagtggatctgggacagatttcactctcaccatcagcagcctgcaggccgaagatctggcagactattactgtcaacagtacagcaattacccttggacgttcggcggcgggaccaaggtggaaatcaaa

SEQ ID NO: 201 - protein, 23 aa, synthetic construct
  REGION 1..23: note = Girentuximab Humanised Vk4 Variable light chain- LFR1 (protein)
DIQMTQSPSSLSASVGDRVTITC

SEQ ID NO: 202 - protein, 15 aa, synthetic construct
  REGION 1..15: note = Girentuximab Humanised Vk4 Variable light chain- LFR2 (protein)
WYQQKPGQAPRLLIY

SEQ ID NO: 203 - protein, 32 aa, synthetic construct
  REGION 1..32: note = Girentuximab Humanised Vk4 Variable light chain- LFR3 (protein)
GVPDRFSGSGSGTDFTLTISSLQAEDLADYYC

SEQ ID NO: 204 - protein, 10 aa, synthetic construct
  REGION 1..10: note = Girentuximab Humanised Vk4 Variable light chain- LFR4 (protein)
FGGGTKVEIK

SEQ ID NO: 205 - DNA, 69 bp, synthetic construct; identical to SEQ ID NO: 189
  misc_feature 1..69: note = Girentuximab Humanised Vk4 Variable light chain - LFR1 (DNA)
  source 1..69: mol_type = other DNA

SEQ ID NO: 206 - DNA, 45 bp, synthetic construct; identical to SEQ ID NO: 174
  misc_feature 1..45: note = Girentuximab Humanised Vk4 Variable light chain - LFR2 (DNA)
  source 1..45: mol_type = other DNA

SEQ ID NO: 207 - DNA, 96 bp, synthetic construct
  misc_feature 1..96: note = Girentuximab Humanised Vk4 Variable light chain - LFR3 (DNA)
  source 1..96: mol_type = other DNA
ggggtcccagacaggttcagcggcagtggatctgggacagatttcactctcaccatcagcagcctgcaggccgaagatctggcagactattactgt

SEQ ID NO: 208 - DNA, 30 bp, synthetic construct; identical to SEQ ID NO: 160
  misc_feature 1..30: note = Girentuximab Humanised Vk4 Variable light chain - LFR4 (DNA)
  source 1..30: mol_type = other DNA

SEQ ID NO: 209 - protein, 11 aa, synthetic construct
  REGION 1..11: note = Girentuximab Humanised Vk5 Variable light chain - LCDR1 (protein)
KASQNVVSAVA

SEQ ID NO: 210 - protein, 7 aa, synthetic construct
  REGION 1..7: note = Girentuximab Humanised Vk5 Variable light chain - LCDR2 (protein)
SASNRYT

SEQ ID NO: 211 - protein, 9 aa, synthetic construct
  REGION 1..9: note = Girentuximab Humanised Vk5 Variable light chain - LCDR3 (protein)
QQYSNYPWT

SEQ ID NO: 212 - protein, 107 aa, synthetic construct
  REGION 1..107: note = Girentuximab Humanised Vk5 Variable light chain - VL (protein)
DIQMTQSPSSLSASVGDRVTITCKASQNVVSAVAWYQQKPGQAPRRLIYSASNRYTGVPDRFSGSGSGTDFTLTISSLQAEDLADYYCQQYSNYPWTFGGGTKVEIK

SEQ ID NO: 213 - DNA, 33 bp, synthetic construct; identical to SEQ ID NO: 149
  misc_feature 1..33: note = Girentuximab Humanised Vk5 Variable light chain - LCDR1 (DNA)
  source 1..33: mol_type = other DNA

SEQ ID NO: 214 - DNA, 21 bp, synthetic construct
  misc_feature 1..21: note = Girentuximab Humanised Vk5 Variable light chain - LCDR2 (DNA)
  source 1..21: mol_type = other DNA
agcgcctccaacaggtacacc

SEQ ID NO: 215 - DNA, 27 bp, synthetic construct
  misc_feature 1..27: note = Girentuximab Humanised Vk5 Variable light chain - LCDR3 (DNA)
  source 1..27: mol_type = other DNA
caacagtacagcaattacccttggacg

SEQ ID NO: 216 - DNA, 321 bp, synthetic construct
  misc_feature 1..321: note = Girentuximab Humanised Vk5 Variable light chain - VL (DNA)
  source 1..321: mol_type = other DNA
gacatccagatgacccagtctccatccagcctgtctgcatctgtaggagacagagtcaccatcacttgcaaggcaagtcagaacgtggtgagtgctgtggcctggtatcagcagaaaccagggcaggctcctaggaggctgatctatagcgcctccaacaggtacaccggggtcccagacaggttcagcggcagtggatctgggacagatttcactctcaccatcagcagcctgcaggccgaagatctggcagactattactgtcaacagtacagcaattacccttggacgttcggcggcgggaccaaggtggaaatcaaa

SEQ ID NO: 217 - protein, 23 aa, synthetic construct
  REGION 1..23: note = Girentuximab Humanised Vk5 Variable light chain - LFR1 (protein)
DIQMTQSPSSLSASVGDRVTITC

SEQ ID NO: 218 - protein, 15 aa, synthetic construct
  REGION 1..15: note = Girentuximab Humanised Vk5 Variable light chain - LFR2 (protein)
WYQQKPGQAPRRLIY

SEQ ID NO: 219 - protein, 32 aa, synthetic construct; identical to SEQ ID NO: 203
  REGION 1..32: note = Girentuximab Humanised Vk5 Variable light chain - LFR3 (protein)

SEQ ID NO: 220 - protein, 10 aa, synthetic construct
  REGION 1..10: note = Girentuximab Humanised Vk5 Variable light chain - LFR4 (protein)
FGGGTKVEIK

SEQ ID NO: 221 - DNA, 69 bp, synthetic construct; identical to SEQ ID NO: 189
  misc_feature 1..69: note = Girentuximab Humanised Vk5 Variable light chain - LFR1 (DNA)
  source 1..69: mol_type = other DNA

SEQ ID NO: 222 - DNA, 45 bp, synthetic construct
  misc_feature 1..45: note = Girentuximab Humanised Vk5 Variable light chain - LFR2 (DNA)
  source 1..45: mol_type = other DNA
tggtatcagcagaaaccagggcaggctcctaggaggctgatctat

SEQ ID NO: 223 - DNA, 96 bp, synthetic construct; identical to SEQ ID NO: 207
  misc_feature 1..96: note = Girentuximab Humanised Vk5 Variable light chain - LFR3 (DNA)
  source 1..96: mol_type = other DNA

SEQ ID NO: 224 - DNA, 30 bp, synthetic construct; identical to SEQ ID NO: 160
  misc_feature 1..30: note = Girentuximab Humanised Vk5 Variable light chain - LFR4 (DNA)
  source 1..30: mol_type = other DNA

SEQ ID NO: 225 - protein, 330 aa, synthetic construct [sequence omitted]

SEQ ID NO: 226 - protein, 330 aa, synthetic construct
  REGION 1..330: note = Girentuximab modified human IgG1 constant chain region - IgG1 H310A H435Q HC (protein)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNQYTQKSLSLSPGK

SEQ ID NO: 227 - protein, 327 aa, synthetic construct
  REGION 1..327: note = Girentuximab modified human IgG4 constant chain region - IgG4 S228P L235E HC (protein)
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 228 - protein, 327 aa, synthetic construct
  REGION 1..327: note = Girentuximab modified human IgG4 constant chain region - IgG4 S228P L235E H310A H435Q HC (protein)
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLAQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNQYTQKSLSLSLGK

SEQ ID NO: 229 - protein, 107 aa, synthetic construct [sequence omitted]

SEQ ID NO: 230 - protein, 449 aa, synthetic construct
  REGION 1..449: note = Girentuximab native IgG1 allotype Gm (1,17) VH4 heavy chain - IgG1 VH4 heavy chain (protein)
EVQLVESGGGLVKPGGSLRLSCAASGFTFSNYYMSWVRQAPGKGLELVAAINSDGGITYYLDTVKGRFTISRDNAKNTLYLQMNSLRAEDTALYYCARHRSGYFSMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 231 - protein, 449 aa, synthetic construct
  REGION 1..449: note = hGirentuximab IgG1 allotype Gm (1,17) RADmAb VH4 heavy chain - FcRn-null IgG1 allotype H130A H435Q (protein)
EVQLVESGGGLVKPGGSLRLSCAASGFTFSNYYMSWVRQAPGKGLELVAAINSDGGITYYLDTVKGRFTISRDNAKNTLYLQMNSLRAEDTALYYCARHRSGYFSMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNQYTQKSLSLSPGK

SEQ ID NO: 232 - protein, 446 aa, synthetic construct
  REGION 1..446: note = hGirentuximab IgG4 RADmAb VH4 heavy chain - FcR?-null IgG4 S228P L235E (protein)
EVQLVESGGGLVKPGGSLRLSCAASGFTFSNYYMSWVRQAPGKGLELVAAINSDGGITYYLDTVKGRFTISRDNAKNTLYLQMNSLRAEDTALYYCARHRSGYFSMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 233 - protein, 446 aa, synthetic construct
  REGION 1..446: note = hGirentuximab IgG4 RADmAb VH4 heavy chain - FcRn+FcR?-null IgG4 S228P L235E H310A H435Q (protein)
EVQLVESGGGLVKPGGSLRLSCAASGFTFSNYYMSWVRQAPGKGLELVAAINSDGGITYYLDTVKGRFTISRDNAKNTLYLQMNSLRAEDTALYYCARHRSGYFSMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLAQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNQYTQKSLSLSLGK

SEQ ID NO: 234 - protein, 214 aa, synthetic construct
  REGION 1..214: note = hGirentuximab V? Light Chain - V? Light Chain (protein)
DIQMTQSPSSLSASVGDRVTITCKASQNVVSAVAWYQQKPGQAPRLLIYSASNRYTGVPDRFSGSGSGTDFTLTISSLQAEDLADYYCQQYSNYPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 235 - protein, 330 aa, synthetic construct
  REGION 1..330: note = ANT4044/ANT4044-A2 unmodified human IgG1 heavy chain constant region - IgG1 HC (protein)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 236 - protein, 330 aa, synthetic construct
  REGION 1..330: note = ANT4044/ANT4044-A2 modified human IgG1 heavy chain constant region - IgG1 H310A H435Q HC (protein)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNQYTQKSLSLSPGK

SEQ ID NO: 237 - protein, 327 aa, synthetic construct; identical to SEQ ID NO: 228
  REGION 1..327: note = ANT4044/ANT4044-A2 modified human IgG4 constant chain region - IgG4 S228P L235E H310A H435Q HC (protein)

SEQ ID NO: 238 - protein, 107 aa, synthetic construct [sequence omitted]

SEQ ID NO: 239 - protein, 445 aa, synthetic construct
  REGION 1..445: note = ANT4044 RADmAb IgG1 heavy chain (HuX592r) - FcRn-null, IgG1 allotype G1m(3) H310A H435Q (protein)
EVQLVQSGAEVKKPGASVKVSCKASGYTFTEYTIHWVRQAPGKGLEWIGNINPNNGGTTYNQKFEDRVTITVDKSTSTAYMELSSLRSEDTAVYYCAAGWNFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNQYTQKSLSLSPGK

SEQ ID NO: 240 - protein, 442 aa, synthetic construct
  REGION 1..442: note = ANT4044 RADmAb IgG4 heavy chain - FcRn+FcR Gamma-null, IgG4 S228P L235E H310A H435Q (protein)
EVQLVQSGAEVKKPGASVKVSCKASGYTFTEYTIHWVRQAPGKGLEWIGNINPNNGGTTYNQKFEDRVTITVDKSTSTAYMELSSLRSEDTAVYYCAAGWNFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLAQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNQYTQKSLSLSLGK

SEQ ID NO: 241 - protein, 445 aa, synthetic construct
  REGION 1..445: note = ANT4044-A2 RADmAb IgG1 heavy chain - FcRn-null, IgG1 H310A H435Q (protein)
EVQLVQSGAEVKKPGASVKVSCKASGYTFTEYTIHWVRQAPGKGLEWIGNINPNNGGTTYNQKFEDRVTITVDKSTSTAYMELSSLRSEDTAVYYCAAYWLFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNQYTQKSLSLSPGK

SEQ ID NO: 242 - protein, 442 aa, synthetic construct
  REGION 1..442: note = ANT4044-A2 RADmAb IgG4 heavy chain - FcRn+FcR Gamma-null IgG4 S228P L235E H310A H435Q (protein)
EVQLVQSGAEVKKPGASVKVSCKASGYTFTEYTIHWVRQAPGKGLEWIGNINPNNGGTTYNQKFEDRVTITVDKSTSTAYMELSSLRSEDTAVYYCAAYWLFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLAQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNQYTQKSLSLSLGK

SEQ ID NO: 243 - protein, 214 aa, synthetic construct
  REGION 1..214: note = ANT4044-ANT4044-A2 Vk Light chain - Vk Light chain (protein)
DIQMTQSPSTLSASVGDRVTITCKASQDVGTAVDWYQQKPGQAPKLLIYWASTRHTGVPDRFSGSGSGTDFTLTISRLQPEDFAVYYCQQYNSYPLTFGQGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2024/0342323 A1
(43) Pub. Date: Oct. 17, 2024

(54) ANTIBODIES AGAINST CAIX WITH REDUCED AFFINITY FOR THE NEONATAL FC RECEPTOR

(71) Applicant: TELIX PHARMACEUTICALS (INNOVATIONS) PTY LTD, North Melbourne (AU)

(72) Inventors: Michael Paul WHEATCROFT, North Melbourne (AU); Christian Peter BEHRENBRUCH, North Melbourne (AU)

(21) Appl. No.: 18/646,281

(22) Filed: Apr. 25, 2024

Related U.S. Application Data
(63) Continuation of application No. 17/622,341, filed on Dec. 23, 2021.

(30) Foreign Application Priority Data
Jul. 2, 2019 (AU) 2019902343

(51) Int. Cl. A61K 51/10; A61P 35/00; C07K 16/40

(52) U.S. Cl. CPC A61K 51/1045; A61K 51/1075; A61P 35/00; C07K 16/40; A61K 2121/00

(57) ABSTRACT

The invention relates to anti-CAIX antibodies comprising a heavy chain constant region comprising one or more amino acid substitutions compared to a wild-type IgG, wherein the one or more amino acid substitutions reduce the affinity of the antibody for the neonatal Fc receptor (FcRn), thereby reducing the serum half-life of the modified antibody compared to a wild-type antibody of class IgG. The one or more amino acid modification having the effect of reducing FcRn binding is selected from positions His310, His433, His435, His436, Ile253. Antibodies of the present invention are particularly suited for use in radioimmunotherapy.

### FIELD OF THE INVENTION

The invention relates to antibodies, compositions and methods for producing antibodies, in particular antibodies conjugated to a radioisotope, having reduced serum half-life for use in radioimmunotherapy.

### RELATED APPLICATION

This application claims priority from Australian provisional application AU 2019902343, the entire contents of which are incorporated herein by reference.

### BACKGROUND OF THE INVENTION

Radiotherapy is an important form of tumor therapy. Various methods of radiotherapy have been developed to treat tumors. Among them, radioimmunotherapy (RAIT) is one emerging approach to the provision of radiotherapy. It employs antibodies or antibody fragments to direct radioisotopes to specific tissues and cells, thus enhancing specificity of tumor treatment and reducing toxicity. RAIT further reduces its side effects by using low dose rate radiation.

Radiation damage to healthy tissues and organs is a major problem associated with radiotherapy. Such damage has been primarily attributed to radiation-generated reactive oxygen species which oxidize functionally important biological molecules, such as nucleic acids, carbohydrates, lipids and lipoproteins, and damage tissues and cells. They have been implicated in a variety of biological processes, e.g., antimicrobial defense, inflammation, carcinogenesis and aging. As reflected by body weight loss, myelosuppression and blood cell loss, such as decreased white blood cell (WBC) and platelet counts and hematopoietic toxicity are the most notable consequences of the radiation damage. The toxicity severely limits the radiation dosage of RAIT and reduces the effectiveness of tumor treatment.

A number of methods have been developed to attempt to mitigate the hematopoietic toxicity of radiation. Stem cell transplantation (SCT) and bone marrow transplantation (BMT) are the most frequently used methods. However, these approaches are invasive, expensive and may contribute to longer hospitalization of the individual receiving treatment.

Other methods include using cytokines to stimulate the immune system and hemoregulatory proteins such as HP5b to turn off hematopoiesis during the radiation exposure period. These methods have achieved various degrees of success in combating hematopoietic toxicity in small studies but again, require exposing the patient to further medication and treatment, therefore remain largely unused.

There remains a need for new methods for mitigating the toxicity associated with radioimmunotherapy.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

The present invention provides a modified antibody of class IgG for use in radioimmunotherapy, comprising a heavy chain constant region having one or more amino acid substitutions compared to a wild-type antibody of the class IgG, wherein the one or more amino acid substitutions reduce the affinity of the antibody for the neonatal Fc receptor (FcRn), thereby reducing the serum half-life of the modified antibody compared to a wild-type antibody of class IgG.

In one embodiment, the one or more amino acid substitutions are selected from substitutions in the heavy chain constant region 2 (CH2) of the IgG molecule, reducing the affinity of the IgG molecule for FcRn. Alternatively, the one or more amino acid substitutions may be in the heavy chain constant region 3 (CH3) of the IgG molecule, thereby reducing the affinity of the IgG molecule for FcRn. Still further, the amino acid substitutions may include at least one substitution in the CH2 region, and at least one substitution in the CH3 region of the IgG molecule, whereby the substitutions reduce the affinity of the IgG for FcRn.

In certain preferred embodiments, the one or more amino acid substitutions may be at one or more of residues His310, His433, His435, His436, or Ile253 of IgG. Preferably, the amino acid substitutions comprise a substitution in the heavy chain constant region at positions His310 or at His435. More preferably, the amino acid substitutions that reduce the affinity of the antibody for FcRn are at both His310 and His435.

In certain embodiments, the modified antibody retains the ability to bind to one or more Fc-gamma receptors and accordingly, in certain embodiments the modified antibody retains the ability to stimulate effector responses (including ADCC).

In alternative embodiments, the one or more amino acid modifications which reduce the affinity for the FcRn receptor also reduce the affinity for the Fc gamma receptors. The modified antibody may further comprise one or more amino acid substitutions compared a wild-type antibody of the class IgG, wherein the amino acid substitutions further reduce the affinity of the antibody for one or more Fc gamma receptors.

In a further embodiment, the modified antibody further comprises one or more amino acid substitutions compared a wild-type antibody of the class IgG, wherein the amino acid substitutions increase the stability of the CH1-CH2 hinge region in the modified antibody compared to a wild-type antibody of the class IgG.

In one embodiment, the modified antibody is conjugated to a diagnostic or therapeutic agent. The diagnostic or therapeutic agent may be conjugated to the antibody directly or indirectly, e.g. by halogenation of amino acid residues. Preferably, the diagnostic or therapeutic agent is indirectly conjugated to the antibody by way of a linker or chelator moiety. In one example, the modified antibody is conjugated to a chelating moiety, selected from the group consisting of: TMT (6,6″-bis[N,N″,N′″-tetra(carboxymethyl)aminomethyl)-4′-(3-amino-4-methoxyphenyl)-2,2′:6′,2″-terpyridine), DOTA (1,4,7,10-tetraazacyclododecane-NN′,N″(N′″-tetraacetic acid), TCMC, DO3A, CB-DO2A, NOTA, Diamsar, DTPA, CHX-A″-DTPA, TETE, Te2A, HBED, DFO, DFOsq and HOPO or other chelating agent as described herein.

In another example, the modified antibody is conjugated to a bifunctional linker, for example, bromoacetyl, thiols, succinimide ester, TFP ester, a maleimide, or using any amine or thiol-modifying chemistry known in the art.

Preferably, the diagnostic or therapeutic agent is a radioisotope. Examples of suitable isotopes include: actinium-225 (²²⁵Ac), astatine-211 (²¹¹At), bismuth-212 and bismuth-213 (²¹²Bi, ²¹³Bi), copper-64 and copper-67 (⁶⁴Cu, ⁶⁷Cu), gallium-67 and gallium-68 (⁶⁷Ga and ⁶⁸Ga), indium-111 (¹¹¹In), iodine-123, -124, -125 or -131 (¹²³I, ¹²⁴I, ¹²⁵I, ¹³¹I) (¹²³ I), lead-212 (²¹²Pb), lutetium-177 (¹⁷⁷Lu), radium-223 (²²³Ra), samarium-153 (¹⁵³Sm), scandium-44 and scandium-47 (⁴⁴Sc, ⁴⁷Sc), strontium-90 (⁹⁰ Sr), technetium-99 (^{99m}Tc), yttrium-86 and yttrium-90 (⁸⁶Y, ⁹⁰Y), zirconium-89 (⁸⁹Zr).

The modified antibody of class IgG with reduced FcRn binding affinity compared to an unmodified antibody of class IgG may be any antibody that is useful for targeting a diagnostic or therapeutic agent to a biological site. The antibody may be of any IgG class, including IgG1 (human or murine), IgG2, IgG4, murine IgG2a. In preferred examples, the antibody is any antibody that is useful for targeting or for delivering a diagnostic or therapeutic agent to a cancer cell. Examples of suitable antibodies include the IgG1 antibodies trastuzumab (Herceptin®), rituximab (Rituxan®), bevacizumab (Avastin®), dinutuximab (Unituxin®), the IgG2 antibody panitumumab (Vectibix®), the IgG4 antibodies, pembrolizumab (Keytruda®), nivolumab (Opdivo®), the murine IgG2a antibody tositumomab (Bexxar®) and the murine IgG1 antibody ibritumomab (Zevalin®). Other examples include gemtuzumab (Mylotarg®), brentuximab (Adcetris®), Inotuzumab (Besponsa®), glembatumumab (CDX-011), anetumab (BAY 94-9343), mirvetuximab (IMGN853) depatuxizumab (ABT-414), rovalpituzumab (Rova-T) and vadastuximab talirine (SGN-CD33A).

The present invention also provides a modified antibody of class IgG with reduced FcRn binding affinity compared to an unmodified antibody of class IgG, or compared to a wild-type antibody of the class IgG comprising:
a heavy chain constant region having one or more amino acid substitutions compared to a wild-type antibody of the class IgG, wherein the one or more amino acid substitutions reduce the affinity of the antibody for the neonatal Fc receptor (FcRn), thereby reducing the serum half-life of the modified antibody compared to a wild-type antibody of class IgG.
wherein said antibody binds specifically to prostate specific membrane antigen (PSMA) and wherein the antibody comprises:
FR1-CDR1-FR2-CDR2-FR3-CDR3-FR4-linker-FR1a-CDR1a-FR2a-CDR2a-FR3a-CDR3a-FR4a
wherein:
FR1, FR2, FR3 and FR4 are each framework regions;
CDR1, CDR2 and CDR3 are each complementarity determining regions;
FR1a, FR2a, FR3a and FR4a are each framework regions;
CDR1a, CDR2a and CDR3a are each complementarity determining regions;
wherein the sequence of any of the complementarity determining regions have an amino acid sequence as described in Table 1 below. Preferably, the framework regions have an amino acid sequence also as described in Table 1 below, including amino acid variation at particular residues which can be determined by aligning the various framework regions derived from each antibody. The invention also includes where CDR1, CDR2 and CDR3 are sequences from the VH, CDR1a, CDR2a and CDR3a are sequences from VL, or where CDR1, CDR2 and CDR3 are sequences from the VL, CDR1a, CDR2a and CDR3a are sequences from VH.

More specifically, the present invention provides a modified antibody of class IgG with reduced FcRn binding affinity compared to an unmodified antibody of class IgG, or compared to a wild-type antibody of the class IgG comprising:
a heavy chain constant region wherein one or more amino acid residues at positions His310, His433, His435, His436, Ile253 are different from the residues present in the unmodified antibody, or a wild-type antibody of the class IgG,
wherein said antibody binds specifically to prostate specific membrane antigen (PSMA) and wherein the antibody comprises:
FR1-CDR1-FR2-CDR2-FR3-CDR3-FR4-linker-FR1a-CDR1a-FR2a-CDR2a-FR3a-CDR3a-FR4a
wherein:
FR1, FR2, FR3 and FR4 are each framework regions;
CDR1, CDR2 and CDR3 are each complementarity determining regions;
FR1a, FR2a, FR3a and FR4a are each framework regions;
CDR1a, CDR2a and CDR3a are each complementarity determining regions;
wherein the sequence of any of the complementarity determining regions have an amino acid sequence as described in Table 1 below. Preferably, the framework regions have an amino acid sequence also as described in Table 1 below, including amino acid variation at particular residues which can be determined by aligning the various framework regions derived from each antibody. The invention also includes where CDR1, CDR2 and CDR3 are sequences from the VH, CDR1a, CDR2a and CDR3a are sequences from VL, or where CDR1, CDR2 and CDR3 are sequences from the VL, CDR1a, CDR2a and CDR3a are sequences from VH.

In one embodiment, the antibody that specifically binds to PSMA comprises an antigen binding site that consists essentially of or consists of an amino acids sequence of (in order of N to C terminus or C to N terminus) SEQ ID NO: 4 or 20.

In a further embodiment, the antibody that specifically binds to PSMA comprises at least one of:
(i) a VH comprising a complementarity determining region (CDR) 1 comprising a sequence at least about 80%, at least 85%, at least 90%, at least 92%, at least 95%, at least 97%, at least 99% identical to a sequence set forth in SEQ ID NO 1 or 17, a CDR2 comprising a sequence at least about 80%, at least 85%, at least 90%, at least 92%, at least 95%, at least 97%, at least 99% identical to a sequence set in SEQ ID NO: 2 or 18, and a CDR3 comprising a sequence at least about 80%, at least 85%, at least 90%, at least 92%, at least 95%, at least 97%, at least 99% identical to a sequence set forth in SEQ ID NO: 3 or 19;
(ii) a VH comprising a sequence at least about 95% or 96% or 97% or 98% or 99% identical to a sequence set forth in SEQ ID NO: 4 or 20;
(iii) a VL comprising a CDR1 comprising a sequence at least about 80%, at least 85%, at least 90%, at least 92%, at least 95%, at least 97%, at least 99% identical to a sequence set forth in SEQ ID NO: 33, a CDR2 comprising a sequence at least about 80%, at least 85%, at least 90%, at least 92%, at least 95%, at least 97%, at least 99% identical to a sequence set forth in SEQ ID NO: 34 and a CDR3 comprising a sequence at least about 80%, at least 85%, at least 90%, at least 92%, at least 95%, at least 97%, at least 99% identical to a sequence set forth in SEQ ID NO: 35;
(iv) a VL comprising a sequence at least about 95% identical to a sequence set forth in SEQ ID NO:36;
(v) a VH comprising a CDR1 comprising a sequence set forth in SEQ ID NO: 1 or 17, a CDR2 comprising a sequence set forth between in SEQ ID NO: 2 or 18 and a CDR3 comprising a sequence set forth in SEQ ID NO: 3 or 19;
(vi) a VH comprising a sequence set forth in SEQ ID NO: 4 or 20;
(vii) a VL comprising a CDR1 comprising a sequence set SEQ ID NO: 33, a CDR2 comprising a sequence set forth in SEQ ID NO: 34 and a CDR3 comprising a sequence set forth in SEQ ID NO: 45;
(viii) a VL comprising a sequence set forth in SEQ ID NO: 36;
(ix) a VH comprising a CDR1 comprising a sequence set forth in SEQ ID NO: 1 or 17, a CDR2 comprising a sequence set forth between in SEQ ID NO: 2 or 18 and a CDR3 comprising a sequence set forth in SEQ ID NO: 3 or 19; and a VL comprising a CDR1 comprising a sequence set SEQ ID NO: 33, a CDR2 comprising a sequence set forth in SEQ ID NO: 34 and a CDR3 comprising a sequence set forth in SEQ ID NO: 35; or
(x) a VH comprising a sequence set forth in SEQ ID NO: 4 or 20 and a VL comprising a sequence set forth in SEQ ID NO: 36.

Preferably, the heavy chain constant region comprises amino acid substitutions at both His310 and His435. The antibody may also comprise amino acid substitutions at residues equivalent to Ser228 and Leu235 of the constant heavy chain region.

In any embodiment, the antibody comprises a heavy chain constant region that comprises the amino acid sequence as set forth in any one of SEQ ID NOs: 235 to 237, preferably wherein the heavy chain constant region comprises the sequence set forth in SEQ ID NO:236.

In still a further embodiment, the heavy chain of the antibody comprises the sequence set forth in any one of SEQ ID NOs: 239 to 242, preferably as set forth in SEQ ID NO: 239.

Still further, in preferred embodiments, the light chain constant region of the antibody comprises the sequence as set forth in SEQ ID NO: 238. More preferably, the antibody comprises a light chain comprising the amino acid sequence as set forth in SEQ ID NO:243.

In a particularly preferred embodiment, the antibody comprises the amino acid sequence set forth in SEQ ID NO: 239 and the sequence set forth in SEQ ID NO: 243.

The present invention also provides a modified antibody of class IgG with reduced FcRn binding affinity compared to that of an unmodified monoclonal antibody of class IgG, or to a wild-type antibody of the class IgG comprising:
a heavy chain constant region having one or more amino acid substitutions compared to a wild-type antibody of the class IgG, wherein the one or more amino acid substitutions reduce the affinity of the antibody for the neonatal Fc receptor (FcRn), thereby reducing the serum half-life of the modified antibody compared to a wild-type antibody of class IgG,
wherein said antibody binds specifically to carbonic anhydrase IX (CAIX) and wherein the antibody comprises:
FR1-CDR1-FR2-CDR2-FR3-CDR3-FR4-linker-FR1a-CDR1a-FR2a-CDR2a-FR3a-CDR3a-FR4a
wherein:
FR1, FR2, FR3 and FR4 are each framework regions;
CDR1, CDR2 and CDR3 are each complementarity determining regions;
FR1a, FR2a, FR3a and FR4a are each framework regions;
CDR1a, CDR2a and CDR3a are each complementarity determining regions;
wherein the sequence of any of the complementarity determining regions have an amino acid sequence as described in Table 2 below. Preferably, the framework regions have an amino acid sequence also as described in Table 2 below, including amino acid variation at particular residues which can be determined by aligning the various framework regions derived from each antibody. The invention also includes where CDR1, CDR2 and CDR3 are sequences from the VH, CDR1a, CDR2a and CDR3a are sequences from VL, or where CDR1, CDR2 and CDR The present invention also provides a modified antibody of class IgG with reduced FcRn binding affinity compared to that of an unmodified monoclonal antibody of class IgG, or to a wild-type antibody of the class IgG comprising:
a heavy chain constant region wherein one or more amino acid residues at positions His310, His433, His435, His436, Ile253 are different from the residues present in the unmodified antibody, or a wild-type antibody of the class IgG,
wherein said antibody binds specifically to carbonic anhydrase IX (CAIX) and wherein the antibody comprises:
FR1-CDR1-FR2-CDR2-FR3-CDR3-FR4-linker-FR1a-CDR1a-FR2a-CDR2a-FR3a-CDR3a-FR4a
wherein:
FR1, FR2, FR3 and FR4 are each framework regions;
CDR1, CDR2 and CDR3 are each complementarity determining regions;
FR1a, FR2a, FR3a and FR4a are each framework regions;
CDR1a, CDR2a and CDR3a are each complementarity determining regions;
wherein the sequence of any of the complementarity determining regions have an amino acid sequence as described in Table 2 below. Preferably, the framework regions have an amino acid sequence also as described in Table 2 below, including amino acid variation at particular residues which can be determined by aligning the various framework regions derived from each antibody. The invention also includes where CDR1, CDR2 and CDR3 are sequences from the VH, CDR1a, CDR2a and CDR3a are sequences from VL, or where CDR1, CDR2 and CDR3 are sequences from the VL, CDR1a, CDR2a and CDR3a are sequences from VH.

In one embodiment, the antibody that specifically binds to CAIX comprises an antigen binding site that consists essentially of or consists of an amino acids sequence of (in order of N to C terminus or C to N terminus) SEQ ID NO: 52, 68, 84, 100 or 116.

In a further embodiment, the antibody that specifically binds to CAIX comprises at least one of:
(i) a VH comprising a complementarity determining region (CDR) 1 comprising a sequence at least about 80%, at least 85%, at least 90%, at least 92%, at least 95%, at least 97%, at least 99% identical to a sequence set forth in SEQ ID NO 49, 65, 81, 97 or 113, a CDR2 comprising a sequence at least about 80%, at least 85%, at least 90%, at least 92%, at least 95%, at least 97%, at least 99% identical to a sequence set in SEQ ID NO:50, 66, 82, 98 or 114, and a CDR3 comprising a sequence at least about 80%, at least 85%, at least 90%, at least 92%, at least 95%, at least 97%, at least 99% identical to a sequence set forth in SEQ ID NO: 51, 67, 83, 99 or 115;
(ii) a VH comprising a sequence at least about 95% or 96% or 97% or 98% or 99% identical to a sequence set forth in SEQ ID NO: 52, 68, 84, 100 or 116;
(iii) a VL comprising a CDR1 comprising a sequence at least about 80%, at least 85%, at least 90%, at least 92%, at least 95%, at least 97%, at least 99% identical to a sequence set forth in SEQ ID NO: 129, 145, 161, 177, 193, or 209, a CDR2 comprising a sequence at least about 80%, at least 85%, at least 90%, at least 92%, at least 95%, at least 97%, at least 99% identical to a sequence set forth in SEQ ID NO: 130, 146, 162, 178, 194 or 210 and a CDR3 comprising a sequence at least about 80%, at least 85%, at least 90%, at least 92%, at least 95%, at least 97%, at least 99% identical to a sequence set forth in SEQ ID NO: 131, 147, 163, 179, 195, or 211;
(iv) a VL comprising a sequence at least about 95% identical to a sequence set forth in SEQ ID NO:132, 148, 164, 180, 196 or 212;
(v) a VH comprising a CDR1 comprising a sequence set forth in SEQ ID NO: 49, 65, 81, 97 or 113, a CDR2 comprising a sequence set forth between in SEQ ID NO: 50, 66, 82, 98 or 114 and a CDR3 comprising a sequence set forth in SEQ ID NO: 51, 67, 83, 99 or 115;
(vi) a VH comprising a sequence set forth in SEQ ID NO: 52, 68, 84, 100 or 116;
(vii) a VL comprising a CDR1 comprising a sequence set SEQ ID NO: 129, 145, 161, 177, 193, or 209, a CDR2 comprising a sequence set forth in SEQ ID NO: 130, 146, 162, 178, 194, or 210 and a CDR3 comprising a sequence set forth in SEQ ID NO: 131, 147, 163, 179, 195 or 211;
(viii) a VL comprising a sequence set forth in SEQ ID NO: 132, 148, 164, 180, 196 or 212;
(ix) a VH comprising a CDR1 comprising a sequence set forth in SEQ ID NO: 49, 65, 81, 97 or 113, a CDR2 comprising a sequence set forth between in SEQ ID NO: 50, 66, 82, 98 or 114 and a CDR3 comprising a sequence set forth in SEQ ID NO: 51, 67, 83, 99 or 115; and a VL comprising a CDR1 comprising a sequence set SEQ ID NO: 129, 145, 161, 177, 193, or 209, a CDR2 comprising a sequence set forth in SEQ ID NO: 130, 146, 162, 178, 194, or 210 and a CDR3 comprising a sequence set forth in SEQ ID NO: 131, 147, 163, 179, 195 or 211; or
(x) a VH comprising a sequence set forth in SEQ ID NO: 52, 68, 84, 100 or 116 and a VL comprising a sequence set forth in SEQ ID NO: 132, 148, 164, 180, 196 or 212.

Preferably, the heavy chain constant region comprises amino acid substitutions at both His310 and His435. The antibody may also comprise amino acid substitutions at residues equivalent to Ser228 and Leu235 of the constant heavy chain region.

Preferably, the antibody comprises a heavy chain constant region comprising the sequence as set forth in any one of SEQ ID NOs:225 to 228, preferably as set forth in SEQ ID NO: 226.

In a still further embodiment, the antibody preferably comprises a heavy chain comprising the sequence set forth in any one of SEQ ID NOs:230 to 233, preferably as set forth in SEQ ID NO: 231.

In any embodiment, the antibody comprises a light chain constant region comprising the amino acid sequence as set forth in SEQ ID NO:229. Preferably, the antibody comprises a light chain comprising the amino acid sequence as set forth in SEQ ID NO:234.

In a particular preferred embodiment, the antibody comprises the sequence set forth in SEQ ID NO:231 and the sequence set forth in SEQ ID NO: 234.

The present invention also provides a molecule comprising an immunoglobulin moiety and a non-protein agent conjugated thereto,
wherein, the immunoglobulin moiety specifically binds to a tumour associated antigen,
wherein the immunoglobulin moiety has reduced or abolished affinity for the FcRn receptor compared to a wild-type immunoglobulin; and
wherein the non-protein agent comprises a therapeutic moiety such as a cytotoxin or a radioactive element.

The immunoglobulin moiety may comprise any antibody that is useful for binding to a tumour-associated antigen, included but not limited to trastuzumab (Herceptin®), rituximab (Rituxan®), bevacizumab (Avastin®), dinutuximab (Unituxin®), panitumumab (Vectibix®), pembrolizumab (Keytruda®), nivolumab (Opdivo®), tositumomab (Bexxar®), ibritumomab (Zevalin®), gemtuzumab (Mylotarg®), brentuximab (Adcetris®), Inotuzumab (Besponsa®), glembatumumab (CDX-011), anetumab (BAY 94-9343), mirvetuximab (IMGN853) depatuxizumab (ABT-414), rovalpituzumab (Rova-T) and vadastuximab talirine (SGN-CD33A), or any other antibody as described herein.

The present invention provides a molecule comprising an immunoglobulin moiety and a non-protein agent conjugated thereto,
wherein, the immunoglobulin moiety specifically binds to PSMA and comprises an antigen binding site including:
FR1-CDR1-FR2-CDR2-FR3-CDR3-FR4-linker-FR1a-CDR1a-FR2a-CDR2a-FR3a-CDR3a-FR4a
wherein:
FR1, FR2, FR3 and FR4 are each framework regions;
CDR1, CDR2 and CDR3 are each complementarity determining regions;
FR1a, FR2a, FR3a and FR4a are each framework regions;
CDR1a, CDR2a and CDR3a are each complementarity determining regions;
wherein the sequence of any of the complementarity determining regions have an amino acid sequence as described in Table 1 below;
wherein the immunoglobulin moiety has reduced or abolished affinity for the FcRn receptor compared to a wild-type immunoglobulin; and
wherein the non-protein agent comprises a therapeutic moiety such as a cytotoxin or a radioactive element.

Preferably, the framework regions have an amino acid sequence also as described in Table 1 below, including amino acid variation at particular residues which can be determined by aligning the various framework regions derived from each antibody. The invention also includes where CDR1, CDR2 and CDR3 are sequences from the VH, CDR1a, CDR2a and CDR3a are sequences from VL, or where CDR1, CDR2 and CDR3 are sequences from the VL, CDR1a, CDR2a and CDR3a are sequences from VH. Preferably, the immunoglobulin moiety has amino acid substitutions at residues equivalent to His310 and/or His435 in the constant heavy chain region. The immunoglobulin moiety may also comprise amino acid substitutions at residues equivalent to Ser228 and Leu235 of the constant heavy chain region.

Preferably, the non-protein agent comprises a radioactive element.

The present invention also provides a molecule comprising an immunoglobulin moiety and a non-protein agent conjugated thereto,
wherein, the immunoglobulin moiety specifically binds to PSMA and comprises: an antigen binding site that consists essentially of or consists of an amino acids sequence of (in order of N to C terminus or C to N terminus) SEQ ID NO: 4 or 20;
wherein the immunoglobulin moiety has reduced or abolished affinity for the FcRn receptor compared to a wild-type immunoglobulin; and
wherein the non-protein agent comprises a therapeutic moiety such as a cytotoxin or a radioactive element.

Preferably, the immunoglobulin moiety has amino acid substitutions at residues equivalent to His310 and/or His435 in the constant heavy chain region. The immunoglobulin moiety may also comprise amino acid substitutions at residues equivalent to Ser228 and Leu235 of the constant heavy chain region.

The present invention also provides a molecule comprising an immunoglobulin moiety and a non-protein agent conjugated thereto,
wherein, the immunoglobulin moiety has reduced or abolished affinity for the FcRn receptor compared to a wild-type immunoglobulin and wherein the immunoglobulin moiety specifically binds to PSMA and comprises at least one of:
(i) a VH comprising a complementarity determining region (CDR) 1 comprising a sequence at least about 80%, at least 85%, at least 90%, at least 92%, at least 95%, at least 97%, at least 99% identical to a sequence set forth in SEQ ID NO 1 or 17, a CDR2 comprising a sequence at least about 80%, at least 85%, at least 90%, at least 92%, at least 95%, at least 97%, at least 99% identical to a sequence set in SEQ ID NO: 2 or 18, and a CDR3 comprising a sequence at least about 80%, at least 85%, at least 90%, at least 92%, at least 95%, at least 97%, at least 99% identical to a sequence set forth in SEQ ID NO: 3 or 19;
(ii) a VH comprising a sequence at least about 95% or 96% or 97% or 98% or 99% identical to a sequence set forth in SEQ ID NO: 4 or 20;
(iii) a VL comprising a CDR1 comprising a sequence at least about 80%, at least 85%, at least 90%, at least 92%, at least 95%, at least 97%, at least 99% identical to a sequence set forth in SEQ ID NO: 33, a CDR2 comprising a sequence at least about 80%, at least 85%, at least 90%, at least 92%, at least 95%, at least 97%, at least 99% identical to a sequence set forth in SEQ ID NO: 34 and a CDR3 comprising a sequence at least about 80%, at least 85%, at least 90%, at least 92%, at least 95%, at least 97%, at least 99% identical to a sequence set forth in SEQ ID NO: 35;
(iv) a VL comprising a sequence at least about 95% identical to a sequence set forth in SEQ ID NO:36;
(v) a VH comprising a CDR1 comprising a sequence set forth in SEQ ID NO: 1 or 17, a CDR2 comprising a sequence set forth between in SEQ ID NO: 2 or 18 and a CDR3 comprising a sequence set forth in SEQ ID NO: 3 or 19;
(vi) a VH comprising a sequence set forth in SEQ ID NO: 4 or 20;
(vii) a VL comprising a CDR1 comprising a sequence set SEQ ID NO: 33, a CDR2 comprising a sequence set forth in SEQ ID NO: 34 and a CDR3 comprising a sequence set forth in SEQ ID NO: 45;
(viii) a VL comprising a sequence set forth in SEQ ID NO: 36;
(ix) a VH comprising a CDR1 comprising a sequence set forth in SEQ ID NO: 1 or 17, a CDR2 comprising a sequence set forth between in SEQ ID NO: 2 or 18 and a CDR3 comprising a sequence set forth in SEQ ID NO: 3 or 19; and a VL comprising a CDR1 comprising a sequence set SEQ ID NO: 33, a CDR2 comprising a sequence set forth in SEQ ID NO: 34 and a CDR3 comprising a sequence set forth in SEQ ID NO: 35; or
(x) a VH comprising a sequence set forth in SEQ ID NO: 4 or 20 and a VL comprising a sequence set forth in SEQ ID NO: 36.

Preferably, the immunoglobulin moiety has amino acid substitutions at residues equivalent to His310 and/or His435 in the constant heavy chain region. The immunoglobulin moiety may also comprise amino acid substitutions at residues equivalent to Ser228 and Leu235 of the constant heavy chain region. Preferably, the non-protein agent comprises a radioactive element.

In any embodiment, the immunoglobulin comprises a heavy chain constant region that comprises the amino acid sequence as set forth in any one of SEQ ID NOs: 235 to 237, preferably wherein the heavy chain constant region comprises the sequence set forth in SEQ ID NO:236.

In still a further embodiment, the immunoglobulin comprises the sequence set forth in any one of SEQ ID NOs:239 to 242, preferably 239.

In any embodiment, the immunoglobulin comprises a light chain constant region comprising the sequence of SEQ ID NO:238. In an embodiment the immunoglobulin comprises a light chain having the amino acid sequence as set forth in SEQ ID NO:243.

In a particularly preferred embodiment, the immunoglobulin comprises the sequences set forth in SEQ ID NOs:239 and 243.

The present invention also provides a molecule comprising an immunoglobulin moiety and a non-protein agent conjugated thereto,
wherein, the immunoglobulin moiety specifically binds to CAIX and comprises an antigen binding site including:
FR1-CDR1-FR2-CDR2-FR3-CDR3-FR4-linker-FR1a-CDR1a-FR2a-CDR2a-FR3a-CDR3a-FR4a
wherein:
FR1, FR2, FR3 and FR4 are each framework regions;
CDR1, CDR2 and CDR3 are each complementarity determining regions;
FR1a, FR2a, FR3a and FR4a are each framework regions;
CDR1a, CDR2a and CDR3a are each complementarity determining regions;
wherein the sequence of any of the complementarity determining regions have an amino acid sequence as described in Table 2 below,
wherein the immunoglobulin moiety has reduced or abolished affinity for the FcRn receptor compared to a wild-type immunoglobulin; and
wherein the non-protein agent comprises a therapeutic moiety such as a cytotoxin or a radioactive element.

Preferably, the framework regions have an amino acid sequence also as described in Table 2 below, including amino acid variation at particular residues which can be determined by aligning the various framework regions derived from each antibody. The invention also includes where CDR1, CDR2 and CDR3 are sequences from the VH, CDR1a, CDR2a and CDR3a are sequences from VL, or where CDR1, CDR2 and CDR3 are sequences from the VL, CDR1a, CDR2a and CDR3a are sequences from VH.

Preferably, the immunoglobulin moiety has amino acid substitutions at residues equivalent to His310 and/or His435 in the constant heavy chain region. The immunoglobulin moiety may also comprise amino acid substitutions at residues equivalent to Ser228 and Leu235 of the constant heavy chain region. Preferably, the non-protein agent comprises a radioactive element.

The present invention also provides a molecule comprising an immunoglobulin moiety and a non-protein agent conjugated thereto,
wherein, the immunoglobulin moiety specifically binds to CAIX and comprises: an antigen binding site that consists essentially of or consists of an amino acids sequence of (in order of N to C terminus or C to N terminus) SEQ ID NO: 52, 68, 84, 100 or 116,
wherein the immunoglobulin moiety has reduced or abolished affinity for the FcRn receptor compared to a wild-type immunoglobulin; and
wherein the non-protein agent comprises a therapeutic moiety such as a cytotoxin or a radioactive element.

Preferably, the immunoglobulin moiety has amino acid substitutions at residues equivalent to His310 and/or His435 in the constant heavy chain region. The immunoglobulin moiety may also comprise amino acid substitutions at residues equivalent to Ser228 and Leu235 of the constant heavy chain region. Preferably, the non-protein agent comprises a radioactive element.

The present invention also provides a molecule comprising an immunoglobulin moiety and a non-protein agent conjugated thereto, wherein, the immunoglobulin moiety has reduced or abolished affinity for the FcRn receptor compared to a wild-type immunoglobulin and specifically binds to CAIX and comprises at least one of:
(i) a VH comprising a complementarity determining region (CDR) 1 comprising a sequence at least about 80%, at least 85%, at least 90%, at least 92%, at least 95%, at least 97%, at least 99% identical to a sequence set forth in SEQ ID NO 49, 65, 81, 97 or 113, a CDR2 comprising a sequence at least about 80%, at least 85%, at least 90%, at least 92%, at least 95%, at least 97%, at least 99% identical to a sequence set in SEQ ID NO:50, 66, 82, 98 or 114, and a CDR3 comprising a sequence at least about 80%, at least 85%, at least 90%, at least 92%, at least 95%, at least 97%, at least 99% identical to a sequence set forth in SEQ ID NO: 51, 67, 83, 99 or 115;
(ii) a VH comprising a sequence at least about 95% or 96% or 97% or 98% or 99% identical to a sequence set forth in SEQ ID NO: 52, 68, 84, 100 or 116;
(iii) a VL comprising a CDR1 comprising a sequence at least about 80%, at least 85%, at least 90%, at least 92%, at least 95%, at least 97%, at least 99% identical to a sequence set forth in SEQ ID NO: 129, 145, 161, 177, 193, or 209, a CDR2 comprising a sequence at least about 80%, at least 85%, at least 90%, at least 92%, at least 95%, at least 97%, at least 99% identical to a sequence set forth in SEQ ID NO: 130, 146, 162, 178, 194 or 210 and a CDR3 comprising a sequence at least about 80%, at least 85%, at least 90%, at least 92%, at least 95%, at least 97%, at least 99% identical to a sequence set forth in SEQ ID NO: 131, 147, 163, 179, 195, or 211;
(iv) a VL comprising a sequence at least about 95% identical to a sequence set forth in SEQ ID NO:132, 148, 164, 180, 196 or 212;
(v) a VH comprising a CDR1 comprising a sequence set forth in SEQ ID NO: 49, 65, 81, 97 or 113, a CDR2 comprising a sequence set forth between in SEQ ID NO: 50, 66, 82, 98 or 114 and a CDR3 comprising a sequence set forth in SEQ ID NO: 51, 67, 83, 99 or 115;
(vi) a VH comprising a sequence set forth in SEQ ID NO: 52, 68, 84, 100 or 116;
(vii) a VL comprising a CDR1 comprising a sequence set SEQ ID NO: 129, 145, 161, 177, 193, or 209, a CDR2 comprising a sequence set forth in SEQ ID NO: 130, 146, 162, 178, 194, or 210 and a CDR3 comprising a sequence set forth in SEQ ID NO: 131, 147, 163, 179, 195 or 211;
(viii) a VL comprising a sequence set forth in SEQ ID NO: 132, 148, 164, 180, 196 or 212;
(ix) a VH comprising a CDR1 comprising a sequence set forth in SEQ ID NO: 49, 65, 81, 97 or 113, a CDR2 comprising a sequence set forth between in SEQ ID NO: 50, 66, 82, 98 or 114 and a CDR3 comprising a sequence set forth in SEQ ID NO: 51, 67, 83, 99 or 115; and a VL comprising a CDR1 comprising a sequence set SEQ ID NO: 129, 145, 161, 177, 193, or 209, a CDR2 comprising a sequence set forth in SEQ ID NO: 130, 146, 162, 178, 194, or 210 and a CDR3 comprising a sequence set forth in SEQ ID NO: 131, 147, 163, 179, 195 or 211; or
(x) a VH comprising a sequence set forth in SEQ ID NO: 52, 68, 84, 100 or 116 and a VL comprising a sequence set forth in SEQ ID NO: 132, 148, 164, 180, 196 or 212.

Preferably, the non-protein agent comprises a radioactive element. Preferably, the immunoglobulin moiety has amino acid substitutions at residues equivalent to His310 and/or His435 in the constant heavy chain region. The immunoglobulin moiety may also comprise amino acid substitutions at residues equivalent to Ser228 and Leu235 of the constant heavy chain region. Preferably, the non-protein agent comprises a radioactive element.

In any embodiment, the immunoglobulin comprises a heavy chain constant region comprising the sequence as set forth in any one of SEQ ID NOs:225 to 228, preferably 226.

In a still further embodiment, the immunoglobulin comprises a heavy chain comprising the sequence set forth in any one of SEQ ID NOs:230 to 233, preferably as set forth in SEQ ID NO: 231.

In any embodiment, the immunoglobulin comprises a light chain constant region comprising a sequence as set forth in SEQ ID NO:229. In an embodiment, the light chain comprises the sequence of SEQ ID NO:234.

In a particular preferred embodiment, the immunoglobulin comprises the amino acid sequences a set forth in SEQ ID NOs:231 and 234.

The present invention also provides a method of treating cancer in an individual, the method comprising administering to an individual in need thereof, a molecule comprising an immunoglobulin moiety and a non-protein agent conjugated as hereinbefore described.

The present invention also provides a method for generating an antibody suitable for use in radioimmunotherapy, the method comprising:
providing an antibody with an antigen binding site that specifically binds to an epitope present on a cell or tissue requiring radioimmunotherapy;
introducing at least one amino acid substitution into the heavy chain constant region of the antibody to generate a modified antibody, wherein the at least one amino acid substitution is selected from the group consisting of amino acid substitutions at residues His310, His435, and Ile253, thereby causing an alteration of the binding affinity for FcRn and/or the serum half-life of said antibody;
conjugating the modified antibody with a radioactive element
thereby generating an antibody suitable for use in radioimmunotherapy.

In certain embodiments, the antibody is an antibody as described herein, including an antibody having any of the complementarity determining regions, framework regions, variable light or variable heavy regions as described in Tables 1 and 2 below. Preferably, the modified antibody also comprises amino acid substitutions at residues equivalent to Ser228 and Leu235 of the constant heavy chain region.

Preferably, the radioactive element is conjugated to the modified antibody using a chelating agent, for example DOTA.

The present invention also provides a method of generating an antibody-radioisotope immunoconjugate for use in the treatment of a disease, the method comprising:
providing an antibody with an antigen binding site that specifically binds to an epitope present on a cell or tissue requiring radioimmunotherapy;
introducing at least one amino acid substitution into the heavy chain constant region of the antibody to generate a modified antibody, wherein the at least one amino acid substitution is selected from the group consisting of substitutions at amino acid residues His310, His435, and Ile253, thereby causing an alteration of the binding affinity for FcRn and/or the serum half-life of said antibody;
conjugating the modified antibody with a radioactive element
thereby generating an antibody-radioisotope immunoconjugate for use in the treatment of a disease.

Preferably, the antibody is an antibody as described herein, including an antibody having any of the complementarity determining regions, framework regions, variable light or variable heavy regions as described in Tables 1 and 2 below. Preferably, the modified antibody also comprises amino acid substitutions at residues equivalent to Ser228 and Leu235 of the constant heavy chain region Preferably, the radioactive element is conjugated to the modified antibody using a chelating agent, for example DOTA. More preferably, the modified antibody comprises amino acid substitutions His310Ala and His435Gln.

Preferably the disease is a cancer, including prostate cancer or renal cell carcinoma.

The present invention further provides for a method of producing a modified antibody with an altered binding affinity for FcRn and/or an altered serum half-life compared with an unmodified form of the antibody, wherein said method comprises:
(a) providing an expression vector (preferably a replicable expression vector) comprising a suitable promoter operably linked to a nucleic acid molecule encoding at least a constant region of an immunoglobulin heavy chain wherein at least one amino acid from the heavy chain constant region selected from the group consisting of amino acid residues His310, His435, and Ile253 is substituted with an amino acid which is different from that present in an unmodified antibody, thereby causing an alteration in FcRn binding affinity and/or serum half-life;
(b) transforming host cells with said vector; and
(c) culturing said transformed host cells to produce said modified antibody.

Optionally, such a method further comprises: preparing a second expression vector (preferably a replicable expression vector) comprising a promoter operably linked to DNA encoding a complementary immunoglobulin light chain and further transforming said cell line with said second vector.

A method for altering the serum half-life of an antibody for use in radioimmunotherapy, the method comprising:
providing an antibody with an antigen binding site that specifically binds to an epitope present on a cell or tissue requiring radioimmunotherapy;
introducing at least one amino acid substitution into the heavy chain constant region of the antibody to generate a modified antibody, wherein the at least one amino acid substitution is selected from the group consisting of
substitutions at amino acid residues His310, His435, and Ile253, thereby causing an alteration of the binding affinity for FcRn and the serum half-life of said antibody.

Preferably, the method further includes conjugating the modified antibody with a radioactive element. Preferably, the antibody is an antibody as described herein, for binding to PSMA or CAIX including an antibody having any of the complementarity determining regions, framework regions, variable light or variable heavy regions as described in Tables 1 and 2 below. Preferably, the modified antibody also comprises amino acid substitutions at residues equivalent to Ser228 and Leu235 of the constant heavy chain region, including Ser228Pro and/or Leu235Glu. More preferably, the modified antibody comprises amino acid substitutions His310Ala and His435Gln.

A method for reducing the toxicity of an antibody for use in radioimmunotherapy, the method comprising
providing an antibody with an antigen binding site that specifically binds to an epitope present on a cell or tissue requiring radioimmunotherapy;
introducing at least one amino acid substitution into the heavy chain constant region of the antibody to generate a modified antibody, wherein the at least one amino acid substitution is selected from the group consisting of substitutions at amino acid residues His310, His435, and Ile253, wherein the amino acid substitutions reduce the serum half-life and/or increase the clearance of the modifies antibody from the circulation,
thereby reducing the toxicity of the antibody when it is conjugated to a radioactive element for use in radioimmunotherapy.

In any embodiment, reducing the toxicity of an antibody includes reducing a number of toxic effects which would otherwise result from longer-term residence of radioisotope in the circulation (including haematological toxicity, absorption into bone and bone marrow irradiation).

In any embodiment, the toxicity of a radio-labelled antibody or radioimmunoconjugate herein described is assessed by determining the tumour:blood ratio of the antibody or immunoconjugate following administration to an individual.

In any embodiment of the invention, the tumour:blood ratio of the modified antibodies of the present invention is at least 2 times greater, at least 3 times greater, at least 4 times greater, at least 6 times greater, at least 8 times greater or at least 10 times greater, or more, than for unmodified antibodies that do not have the modifications to the heavy chain constant region as herein described, when the ratio is determined at least 8 hours following administration of the antibody. Alternatively, the ratio is determined at least 24, 48, 72 or 120 hours following administration of the antibody to an individual. In certain embodiments, the tumour:blood ratio of the modified antibodies of the present invention is at least 50 times greater, at least 100 times greater, at least 200 times greater or at least 300 times greater than for unmodified antibodies that do not have the modifications to the heavy chain constant region as herein described, when the ratio is determined at least 120 hours following administration of the antibody.

In any embodiment of the invention, the modified antibodies herein described having reduced or altered serum half-life compared to unmodified antibodies, have a serum clearance rate that is at least two times faster, at least three times faster or more, than the unmodified antibodies.

In particularly preferred embodiments of the invention, the antibodies described herein are suitable for use in a theranostic pair, wherein the theranostic pair comprises 1) the antibody coupled to an imaging agent and 2) the antibody coupled to an agent for therapy. For example, the antibody may firstly be used as a diagnostic when coupled to a radioisotope suitable for use in radioimaging, Secondly, the antibody may be used as a therapeutic when coupled to a radioisotope or cytotoxic agent suitable for use in therapy.

The present invention also provides an in vivo method of diagnosing, monitoring or prognosing a disease, disorder or infection in a subject comprising:
(a) administering to a subject an effective amount of a modified antibody as herein described, said modified antibody specifically binding to an antigen associated with a disease, disorder or infection;
(b) allowing the modified antibody to concentrate at sites in said subject where said antigen is found; and
(c) detecting said modified antibody;
whereby detection of said modified antibody above a background or standard level indicates that the subject has said disease disorder or infection.

The present invention provides an antigen binding site that binds to or specifically binds to prostate specific membrane antigen (PSMA). Preferably, the antigen binding site of the invention binds to or specifically binds to human PSMA.

The invention provides an antigen binding site for binding to PSMA, the antigen binding site comprising:
FR1-CDR1-FR2-CDR2-FR3-CDR3-FR4-linker-FR1a-CDR1a-FR2a-CDR2a-FR3a-CDR3a-FR4a
wherein:
FR1, FR2, FR3 and FR4 are each framework regions;
CDR1, CDR2 and CDR3 are each complementarity determining regions;
FR1a, FR2a, FR3a and FR4a are each framework regions;
CDR1a, CDR2a and CDR3a are each complementarity determining regions;
wherein the sequence of any of the framework regions or complementarity determining regions are as described herein.

The invention provides an antigen binding site for binding to PSMA, the antigen binding site including:
FR1-CDR1-FR2-CDR2-FR3-CDR3-FR4-linker-FR1a-CDR1a-FR2a-CDR2a-FR3a-CDR3a-FR4a
wherein:
FR1, FR2, FR3 and FR4 are each framework regions;
CDR1, CDR2 and CDR3 are each complementarity determining regions;
FR1a, FR2a, FR3a and FR4a are each framework regions;
CDR1a, CDR2a and CDR3a are each complementarity determining regions;
wherein the sequence of any of the complementarity determining regions have an amino acid sequence as described in Table 1 below. Preferably, the framework regions have an amino acid sequence also as described in Table 1 below, including amino acid variation at particular residues which can be determined by aligning the various framework regions derived from each antibody. The invention also includes where CDR1, CDR2 and CDR3 are sequences from the VH, CDR1a, CDR2a and CDR3a are sequences from VL, or where CDR1, CDR2 and CDR3 are sequences from the VL, CDR1a, CDR2a and CDR3a are sequences from VH.

The invention provides an antigen binding site comprising, consisting essentially of or consisting of an amino acids sequence of (in order of N to C terminus or C to N terminus) SEQ ID NO: 4 or 20.

The present invention also provides an antigen binding site comprising an antigen binding domain of an antibody, wherein the antigen binding domain binds to or specifically binds to PSMA, wherein the antigen binding domain comprises at least one of:
(i) a VH comprising a complementarity determining region (CDR) 1 comprising a sequence at least about 80%, at least 85%, at least 90%, at least 92%, at least 95%, at least 97%, at least 99% identical to a sequence set forth in SEQ ID NO 1 or 17, a CDR2 comprising a sequence at least about 80%, at least 85%, at least 90%, at least 92%, at least 95%, at least 97%, at least 99% identical to a sequence set in SEQ ID NO: 2 or 18, and a CDR3 comprising a sequence at least about 80%, at least 85%, at least 90%, at least 92%, at least 95%, at least 97%, at least 99% identical to a sequence set forth in SEQ ID NO: 3 or 19;
(ii) a VH comprising a sequence at least about 95% or 96% or 97% or 98% or 99% identical to a sequence set forth in SEQ ID NO: 4 or 20;
(iii) a VL comprising a CDR1 comprising a sequence at least about 80%, at least 85%, at least 90%, at least 92%, at least 95%, at least 97%, at least 99% identical to a sequence set forth in SEQ ID NO: 33, a CDR2 comprising a sequence at least about 80%, at least 85%, at least 90%, at least 92%, at least 95%, at least 97%, at least 99% identical to a sequence set forth in SEQ ID NO: 34 and a CDR3 comprising a sequence at least about 80%, at least 85%, at least 90%, at least 92%, at least 95%, at least 97%, at least 99% identical to a sequence set forth in SEQ ID NO: 35;
(iv) a VL comprising a sequence at least about 95% identical to a sequence set forth in SEQ ID NO:36;
(v) a VH comprising a CDR1 comprising a sequence set forth in SEQ ID NO: 1 or 17, a CDR2 comprising a sequence set forth between in SEQ ID NO: 2 or 18 and a CDR3 comprising a sequence set forth in SEQ ID NO: 3 or 19;
(vi) a VH comprising a sequence set forth in SEQ ID NO: 4 or 20;
(vii) a VL comprising a CDR1 comprising a sequence set SEQ ID NO: 33, a CDR2 comprising a sequence set forth in SEQ ID NO: 34 and a CDR3 comprising a sequence set forth in SEQ ID NO: 45;
(viii) a VL comprising a sequence set forth in SEQ ID NO: 36;
(ix) a VH comprising a CDR1 comprising a sequence set forth in SEQ ID NO: 1 or 17, a CDR2 comprising a sequence set forth between in SEQ ID NO: 2 or 18 and a CDR3 comprising a sequence set forth in SEQ ID NO: 3 or 19; and a VL comprising a CDR1 comprising a sequence set SEQ ID NO: 33, a CDR2 comprising a sequence set forth in SEQ ID NO: 34 and a CDR3 comprising a sequence set forth in SEQ ID NO: 35; or
(x) a VH comprising a sequence set forth in SEQ ID NO: 4 or 20 and a VL comprising a sequence set forth in SEQ ID NO: 36.

In any aspect of the invention, the antigen binding domain further comprises at least one of:
(i) a VH comprising a framework region (FR) 1 comprising a sequence at least about 80%, at least 85%, at least 90%, at least 92%, at least 95%, at least 97%, at least 99% identical to a sequence set forth in SEQ ID NO: 9 or 25, a FR2 comprising a sequence at least about 80%, at least 85%, at least 90%, at least 92%, at least 95%, at least 97%, at least 99% identical to a sequence set in SEQ ID NO10 or 26, a FR3 comprising a sequence at least about 80%, at least 85%, at least 90%, at least 92%, at least 95%, at least 97%, at least 99% identical to a sequence set forth in SEQ ID NO:11 or 27, and a FR4 comprising a sequence at least about 80%, at least 85%, at least 90%, at least 92%, at least 95%, at least 97%, at least 99% identical to a sequence set forth in SEQ ID NO: 12 or 28;
(ii) a VL comprising a FR1 comprising a sequence at least about 80%, at least 85%, at least 90%, at least 92%, at least 95%, at least 97%, at least 99% identical to a sequence set forth in SEQ ID NO: 41, a FR2 comprising a sequence at least about 80%, at least 85%, at least 90%, at least 92%, at least 95%, at least 97%, at least 99% identical to a sequence set forth in SEQ ID NO: 42, a FR3 comprising a sequence at least about 80%, at least 85%, at least 90%, at least 92%, at least 95%, at least 97%, at least 99% identical to a sequence set forth in SEQ ID NO:43, and a FR4 comprising a sequence at least about 80%, at least 85%, at least 90%, at least 92%, at least 95%, at least 97%, at least 99% identical to a sequence set forth in SEQ ID NO: 44;
(iii) a VH comprising a FR1 comprising a sequence set forth in SEQ ID NO: 9 or 25, a FR2 comprising a sequence set forth between in SEQ ID NO: 10 or 26, a FR3 comprising a sequence set forth in SEQ ID NO: 11 or 27, and a FR4 comprising a sequence set forth in SEQ ID NO: 12 or 28;
(iv) a VL comprising a FR1 comprising a sequence set forth in SEQ ID NO: 41, a FR2 comprising a sequence set forth between in SEQ ID NO: 42, a FR3 comprising a sequence set forth in SEQ ID NO: 43, and a FR4 comprising a sequence set forth in SEQ ID NO: 44; or
(v) a VH comprising a FR1 comprising a sequence set forth in SEQ ID NO: 9 or 25, a FR2 comprising a sequence set forth between in SEQ ID NO: 10 or 26, a FR3 comprising a sequence set forth in SEQ ID NO: 11 or 27, and a FR4 comprising a sequence set forth in SEQ ID NO: 12 or 28; and a VL comprising a FR1 comprising a sequence set forth in SEQ ID NO: 41, a FR2 comprising a sequence set forth between in SEQ ID NO: 42, a FR3 comprising a sequence set forth in SEQ ID NO: 43, and a FR4 comprising a sequence set forth in SEQ ID NO: 44.

In any embodiment, the antigen binding site comprises a heavy chain constant region that comprises the amino acid sequence as set forth in any one of SEQ ID NOs: 235 to 237, preferably wherein the heavy chain constant region comprises the sequence set forth in SEQ ID NO:236.

In still a further embodiment, the antigen binding site comprises the sequence set forth in any one of SEQ ID NOs:239 to 242, preferably 239.

In any embodiment, the antigen binding site comprises a light chain constant region comprising the sequence of SEQ ID NO:238. In an embodiment the light chain of the antigen binding site comprises the sequence of SEQ ID NO:243.

In a particularly preferred embodiment, the antigen binding site comprises the sequences set forth in SEQ ID NOs:239 and 243.

The present invention also provides an antigen binding site that binds to or specifically binds to carbonic anhydrase IX (CAIX). Preferably, the antigen binding site of the invention binds to or specifically binds to human CAIX.

The invention provides an antigen binding site for binding to CAIX, the antigen binding site comprising:
FR1-CDR1-FR2-CDR2-FR3-CDR3-FR4-linker-FR1a-CDR1a-FR2a-CDR2a-FR3a-CDR3a-FR4a
wherein:
FR1, FR2, FR3 and FR4 are each framework regions;
CDR1, CDR2 and CDR3 are each complementarity determining regions;
FR1a, FR2a, FR3a and FR4a are each framework regions;
CDR1a, CDR2a and CDR3a are each complementarity determining regions;
wherein the sequence of any of the framework regions or complementarity determining regions are as described herein.

The invention provides an antigen binding site for binding to CAIX, the antigen binding site including:
FR1-CDR1-FR2-CDR2-FR3-CDR3-FR4-linker-FR1a-CDR1a-FR2a-CDR2a-FR3a-CDR3a-FR4a
wherein:
FR1, FR2, FR3 and FR4 are each framework regions;
CDR1, CDR2 and CDR3 are each complementarity determining regions;
FR1a, FR2a, FR3a and FR4a are each framework regions;
CDR1a, CDR2a and CDR3a are each complementarity determining regions;
wherein the sequence of any of the complementarity determining regions have an amino acid sequence as described in Table 2 below. Preferably, the framework regions have an amino acid sequence also as described in Table 2 below, including amino acid variation at particular residues which can be determined by aligning the various framework regions derived from each antibody. The invention also includes where CDR1, CDR2 and CDR3 are sequences from the VH, CDR1a, CDR2a and CDR3a are sequences from VL, or where CDR1, CDR2 and CDR3 are sequences from the VL, CDR1a, CDR2a and CDR3a are sequences from VH.

The invention provides an antigen binding site comprising, consisting essentially of or consisting of an amino acids sequence of (in order of N to C terminus or C to N terminus) SEQ ID NO: 52, 68, 84, 100 or 116.

The present invention also provides an antigen binding site comprising an antigen binding domain of an antibody, wherein the antigen binding domain binds to or specifically binds to CAIX, wherein the antigen binding domain comprises at least one of:
(i) a VH comprising a complementarity determining region (CDR) 1 comprising a sequence at least about 80%, at least 85%, at least 90%, at least 92%, at least 95%, at least 97%, at least 99% identical to a sequence set forth in SEQ ID NO 49, 65, 81, 97 or 113, a CDR2 comprising a sequence at least about 80%, at least 85%, at least 90%, at least 92%, at least 95%, at least 97%, at least 99% identical to a sequence set in SEQ ID NO:50, 66, 82, 98 or 114, and a CDR3 comprising a sequence at least about 80%, at least 85%, at least 90%, at least 92%, at least 95%, at least 97%, at least 99% identical to a sequence set forth in SEQ ID NO: 51, 67, 83, 99 or 115;
(ii) a VH comprising a sequence at least about 95% or 96% or 97% or 98% or 99% identical to a sequence set forth in SEQ ID NO: 52, 68, 84, 100 or 116;
(iii) a VL comprising a CDR1 comprising a sequence at least about 80%, at least 85%, at least 90%, at least 92%, at least 95%, at least 97%, at least 99% identical to a sequence set forth in SEQ ID NO: 129, 145, 161, 177, 193, or 209, a CDR2 comprising a sequence at least about 80%, at least 85%, at least 90%, at least 92%, at least 95%, at least 97%, at least 99% identical to a sequence set forth in SEQ ID NO: 130, 146, 162, 178, 194 or 210 and a CDR3 comprising a sequence at least about 80%, at least 85%, at least 90%, at least 92%, at least 95%, at least 97%, at least 99% identical to a sequence set forth in SEQ ID NO: 131, 147, 163, 179, 195, or 211;
(iv) a VL comprising a sequence at least about 95% identical to a sequence set forth in SEQ ID NO:132, 148, 164, 180, 196 or 212;
(v) a VH comprising a CDR1 comprising a sequence set forth in SEQ ID NO: 49, 65, 81, 97 or 113, a CDR2 comprising a sequence set forth between in SEQ ID NO: 50, 66, 82, 98 or 114 and a CDR3 comprising a sequence set forth in SEQ ID NO: 51, 67, 83, 99 or 115;
(vi) a VH comprising a sequence set forth in SEQ ID NO: 52, 68, 84, 100 or 116;
(vii) a VL comprising a CDR1 comprising a sequence set SEQ ID NO: 129, 145, 161, 177, 193, or 209, a CDR2 comprising a sequence set forth in SEQ ID NO: 130, 146, 162, 178, 194, or 210 and a CDR3 comprising a sequence set forth in SEQ ID NO: 131, 147, 163, 179, 195 or 211;
(viii) a VL comprising a sequence set forth in SEQ ID NO: 132, 148, 164, 180, 196 or 212;
(ix) a VH comprising a CDR1 comprising a sequence set forth in SEQ ID NO: 49, 65, 81, 97 or 113, a CDR2 comprising a sequence set forth between in SEQ ID NO: 50, 66, 82, 98 or 114 and a CDR3 comprising a sequence set forth in SEQ ID NO: 51, 67, 83, 99 or 115; and a VL comprising a CDR1 comprising a sequence set SEQ ID NO: 129, 145, 161, 177, 193, or 209, a CDR2 comprising a sequence set forth in SEQ ID NO: 130, 146, 162, 178, 194, or 210 and a CDR3 comprising a sequence set forth in SEQ ID NO: 131, 147, 163, 179, 195 or 211; or
(x) a VH comprising a sequence set forth in SEQ ID NO: 52, 68, 84, 100 or 116 and a VL comprising a sequence set forth in SEQ ID NO: 132, 148, 164, 180, 196 or 212.

In any aspect of the invention, the antigen binding domain further comprises at least one of:
(i) a VH comprising a framework region (FR) 1 comprising a sequence at least about 80%, at least 85%, at least 90%, at least 92%, at least 95%, at least 97%, at least 99% identical to a sequence set forth in SEQ ID NO:57, 73, 89, 105, or 124, a FR2 comprising a sequence at least about 80%, at least 85%, at least 90%, at least 92%, at least 95%, at least 97%, at least 99% identical to a sequence set in SEQ ID NO: 58, 74, 90, 106, or 122, a FR3 comprising a sequence at least about 80%, at least 85%, at least 90%, at least 92%, at least 95%, at least 97%, at least 99% identical to a sequence set forth in SEQ ID NO:59, 75, 91, 107, or 123, and a FR4 comprising a sequence at least about 80%, at least 85%, at least 90%, at least 92%, at least 95%, at least 97%, at least 99% identical to a sequence set forth in SEQ ID NO: 60, 76, 92, 108, or 124;
(ii) a VL comprising a FR1 comprising a sequence at least about 80%, at least 85%, at least 90%, at least 92%, at least 95%, at least 97%, at least 99% identical to a sequence set forth in SEQ ID NO: 137, 153, 169, 185, 201, or 217, a FR2 comprising a sequence at least about 80%, at least 85%, at least 90%, at least 92%, at least 95%, at least 97%, at least 99% identical to a sequence set forth in SEQ ID NO: 138, 154, 170, 186, 202, or 218, a FR3 comprising a sequence at least about 80%, at least 85%, at least 90%, at least 92%, at least 95%, at least 97%, at least 99% identical to a sequence set forth in SEQ ID NO:139, 155, 171, 187, 203, or 219, and a FR4 comprising a sequence at least about 80%, at least 85%, at least 90%, at least 92%, at least 95%, at least 97%, at least 99% identical to a sequence set forth in SEQ ID NO: 140, 156, 172, 188, 204, or 220;
(iii) a VH comprising a FR1 comprising a sequence set forth in SEQ ID NO: 57, 73, 89, 105, or 124, a FR2 comprising a sequence set forth between in SEQ ID NO: 58, 74, 90, 106, or 122, a FR3 comprising a sequence set forth in SEQ ID NO: 59, 75, 91, 107, or 123, and a FR4 comprising a sequence set forth in SEQ ID NO: 60, 76, 92, 108, or 124;
(iv) a VL comprising a FR1 comprising a sequence set forth in SEQ ID NO: 137, 153, 169, 185, 201, or 217, a FR2 comprising a sequence set forth between in SEQ ID NO: 138, 154, 170, 186, 202, or 218, a FR3 comprising a sequence set forth in SEQ ID NO: 139, 155, 171, 187, 203, or 219, and a FR4 comprising a sequence set forth in SEQ ID NO: 140, 156, 172, 188, 204, or 220; or
(v) a VH comprising a FR1 comprising a sequence set forth in SEQ ID NO: 57, 73, 89, 105, or 124, a FR2 comprising a sequence set forth between in SEQ ID NO: 58, 74, 90, 106, or 122, a FR3 comprising a sequence set forth in SEQ ID NO: 59, 75, 91, 107, or 123, and a FR4 comprising a sequence set forth in SEQ ID NO: 60, 76, 92, 108, or 124; and a VL comprising a FR1 comprising a sequence set forth in SEQ ID NO: 137, 153, 169, 185, 201, or 217, a FR2 comprising a sequence set forth between in SEQ ID NO: 138, 154, 170, 186, 202, or 218, a FR3 comprising a sequence set forth in SEQ ID NO: 139, 155, 171, 187, 203, or 219, and a FR4 comprising a sequence set forth in SEQ ID NO: 140, 156, 172, 188, 204, or 220.

In one embodiment, the VH comprises a sequence at least about 95% or 96% or 97% or 98% or 99% identical to, or comprises a sequence set forth in SEQ ID NO: 84 or 100 and the VL comprises a sequence at least about 95% or 96% or 97% or 98% or 99% identical to, or comprises a sequence set forth in SEQ ID NO: 164, 180 or 196. Preferably, the VH comprises a sequence at least about 95% or 96% or 97% or 98% or 99% identical to, or comprises a sequence set forth in SEQ ID NO: 84 or 100 and the VL comprises a sequence at least about 95% or 96% or 97% or 98% or 99% identical to, or comprises a sequence set forth in SEQ ID NO: 180 or 196. More preferably, the VH comprises a sequence at least about 95% or 96% or 97% or 98% or 99% identical to, or comprises a sequence set forth in SEQ ID NO: 84 and the VL comprises a sequence at least about 95% or 96% or 97% or 98% or 99% identical to, or comprises a sequence set forth in SEQ ID NO: 196. Alternatively, the VH comprises a sequence at least about 95% or 96% or 97% or 98% or 99% identical to, or comprises a sequence set forth in SEQ ID NO: 100 and the VL comprises a sequence at least about 95% or 96% or 97% or 98% or 99% identical to, or comprises a sequence set forth in SEQ ID NO: 180 or 196, preferably 196.

The foregoing antigen binding sites can also be referred to as antigen binding domains of antibodies.

Preferably, an antigen binding site as described herein is an antibody or antigen binding fragment thereof. Typically, the antigen binding site is an antibody, for example, a monoclonal antibody.

As described herein, the antigen binding site may be in the form of:
(i) a single chain Fv fragment (scFv);
(ii) a dimeric scFv (di-scFv);
(iii) one of (i) or (ii) linked to a constant region of an antibody, Fc or a heavy chain constant domain (CH) 2 and/or CH3; or
(iv) one of (i) or (ii) linked to a protein that binds to an immune effector cell.

Further, as described herein, the antigen binding site may be in the form of:
(i) a diabody;
(ii) a triabody;
(iii) a tetrabody;
(iv) a Fab;
(v) a F(ab′)2;
(vi) a Fv;
(vii) one of (i) to (vi) linked to a constant region of an antibody, Fc or a heavy chain constant domain (CH) 2 and/or CH3; or
(viii) one of (i) to (vi) linked to a protein that binds to an immune effector cell.

In any aspect or embodiment, the antibody is a naked antibody. Specifically, the antibody is in a non-conjugated form and is not adapted to form a conjugate.

The invention also provides a fusion protein comprising an antigen binding site, immunoglobulin variable domain, antibody, dab (single domain antibody), di-scFv, scFv, Fab, Fab′, F(ab′)2, Fv fragment, diabody, triabody, tetrabody, linear antibody, single-chain antibody molecule, or multispecific antibody as described herein.

The invention also provides a conjugate in the form of an antigen binding site, immunoglobulin variable domain, antibody, dab, di-scFv, scFv, Fab, Fab′, F(ab′)2, Fv fragment, diabody, triabody, tetrabody, linear antibody, single-chain antibody molecule, or multispecific antibody or fusion protein as described herein conjugated to a label or a cytotoxic agent.

The invention also provides an antibody for binding to an antigen binding site, immunoglobulin variable domain, antibody, dab, di-scFv, scFv, Fab, Fab′, F(ab′)2, Fv fragment, diabody, triabody, tetrabody, linear antibody, single-chain antibody molecule, or multispecific antibody, fusion protein, or conjugate as described herein.

In a preferred embodiment, the antigen binding site is an IgG immunoglobulin comprising one or more amino acid substitutions in the antibody constant domain, CH2-CH3 region, which modify the binding of the antibody to the neonatal Fc receptor (FcRn) relative to a wild-type antibody Fc region. The one or more amino acid modifications change the affinity of the antibody constant domain, Fc region, or FcRn binding fragment thereof, for the FcRn and thereby alter the serum half-life of the antigen binding site.

Preferably the substitution alters the binding affinity for FcRn and/or the serum half-life of said modified antibody relative to the unmodified wild-type antibody. The present invention further provides for a modified antibody having a reduced binding affinity for FcRn and/or a reduced serum half-life as compared with the unmodified antibody, wherein any one or more amino acid residues at positions Ile253 or His310 from the CH2 domain and/or residue His435 from the CH3 domain, is substituted with another amino acid which is different from that present in an unmodified antibody or to an unmodified IgG.

In one example, the one or more amino acid modifications is selected from an amino acid substitution at residue equivalent to H310 and H435. In a further example, the antibody comprises amino acid substitutions at both His310 and His435 residues.

The amino acid substitutions may include substitution from a histidine residue to: alanine, glutamine, glutamic acid or aspartic acid. Preferably, the amino acid substitution at His310 is to alanine. Preferably the amino acid substitution at His435 is to glutamine. Preferably, the amino acid substitution at Ile253 is alanine.

In a further embodiment, the antigen binding site is an antibody that comprises one or more amino acid substitutions which modify the binding of the antibody to activating Fc gamma receptors. The one or more amino acid modifications change the affinity of the antibody constant domain, Fc region, or Fc gamma receptor binding fragment, for any one or more Fc gamma receptors. Preferably, the amino acid modification is at a residue equivalent to Leu235. More preferably, the amino acid modification is from Leu235 to glutamic acid.

In one embodiment, the amino acid modification is a hinge stabilising mutation at Ser228. Preferably the amino acid modification at Ser228 is to proline.

In one embodiment of the invention, the antibody comprises mutations at Ser228, Leu235, His310 and His435. Preferably, the amino acid modifications are Ser228Pro, Leu235Glu, His310Ala and His435Gln.

The amino acid modifications are preferably made in an antibody having an IgG1 isotype, or on an IgG4 isotype.

In a preferred embodiment, the antibody comprises a heavy chain constant region as set forth in any one of SEQ ID NOs:225 to 228 or 235 to 238.

The invention also provides a nucleic acid encoding an antigen binding site, immunoglobulin variable domain, antibody, dab, di-scFv, scFv, Fab, Fab′, F(ab′)2, Fv fragment, diabody, triabody, tetrabody, linear antibody, single-chain antibody molecule, or multispecific antibody, fusion protein or conjugate as described herein.

In one example, such a nucleic acid is included in an expression construct in which the nucleic acid is operably linked to a promoter. Such an expression construct can be in a vector, e.g., a plasmid.

In examples of the invention directed to single polypeptide chain antigen binding sites, the expression construct may comprise a promoter linked to a nucleic acid encoding that polypeptide chain.

In examples directed to multiple polypeptide chains that form an antigen binding site, an expression construct comprises a nucleic acid encoding a polypeptide comprising, e.g., a VH operably linked to a promoter and a nucleic acid encoding a polypeptide comprising, e.g., a VL operably linked to a promoter.

In another example, the expression construct is a bicistronic expression construct, e.g., comprising the following operably linked components in 5′ to 3′ order:
(i) a promoter
(ii) a nucleic acid encoding a first polypeptide;
(iii) an internal ribosome entry site; and
(iv) a nucleic acid encoding a second polypeptide,
wherein the first polypeptide comprises a VH and the second polypeptide comprises a VL, or vice versa.

The present invention also contemplates separate expression constructs one of which encodes a first polypeptide comprising a VH and another of which encodes a second polypeptide comprising a VL. For example, the present invention also provides a composition comprising:
(i) a first expression construct comprising a nucleic acid encoding a polypeptide comprising a VH operably linked to a promoter; and
(ii) a second expression construct comprising a nucleic acid encoding a polypeptide comprising a VL operably linked to a promoter.

The invention provides a cell comprising a vector or nucleic acid described herein. Preferably, the cell is isolated, substantially purified or recombinant. In one example, the cell comprises the expression construct of the invention or:
(i) a first expression construct comprising a nucleic acid encoding a polypeptide comprising a VH operably linked to a promoter; and
(ii) a second expression construct comprising a nucleic acid encoding a polypeptide comprising a VL operably linked to a promoter, wherein the first and second polypeptides associate to form an antigen binding site of the present invention.

Examples of cells of the present invention include bacterial cells, yeast cells, insect cells or mammalian cells.

The invention also provides a pharmaceutical composition comprising an antigen binding site, or comprising a CDR and/or FR sequence as described herein, or an immunoglobulin variable domain, antibody, dab (single domain antibody), di-scFv, scFv, Fab, Fab′, F(ab′)2, Fv fragment, diabody, triabody, tetrabody, linear antibody, single-chain antibody molecule, or multispecific antibody, fusion protein, or conjugate as described herein and a pharmaceutically acceptable carrier, diluent or excipient.

The invention also provides a diagnostic composition comprising an antigen binding site, or comprising a CDR and/or FR sequence as described herein, or antigen binding site, immunoglobulin variable domain, antibody, dab, di-scFv, scFv, Fab, Fab′, F(ab′)2, Fv fragment, diabody, triabody, tetrabody, linear antibody, single-chain antibody molecule, or multispecific antibody, fusion protein or conjugate as described herein, a diluent and optionally a label. Preferably, the antigen binding site is a monoclonal antibody conjugated to a radioisotope.

The invention also provides a kit or article of manufacture comprising an antigen binding site, or comprising a CDR and/or FR sequence as described herein or an immunoglobulin variable domain, antibody, dab, di-scFv, scFv, Fab, Fab′, F(ab′)2, Fv fragment, diabody, triabody, tetrabody, linear antibody, single-chain antibody molecule, or multispecific antibody, fusion protein or conjugate as described herein.

Preferably, the antigen binding site is a monoclonal antibody conjugated to a radioisotope.

An antigen binding site, a protein or antibody as described herein may comprise a human constant region, e.g., an IgG constant region, such as an IgG1, IgG2, IgG3 or IgG4 constant region or mixtures thereof. In the case of an antibody or protein comprising a VH and a VL, the VH can be linked to a heavy chain constant region and the VL can be linked to a light chain constant region.

In one example a protein or antibody as described herein or a composition of a protein or antibody as described herein, comprises a heavy chain constant region, comprising a stabilized heavy chain constant region, comprising a mixture of sequences fully or partially with or without the C-terminal lysine residue.

In one example, an antibody of the invention comprises a VH disclosed herein linked or fused to an IgG4 constant region or stabilized IgG4 constant region (e.g., as discussed above) and the VL is linked to or fused to a kappa light chain constant region.

The functional characteristics of an antigen binding site of the invention will be taken to apply mutatis mutandis to an antibody of the invention.

An antigen binding site as described herein may be purified, substantially purified, isolated and/or recombinant.

The present invention also provides a method for treating or preventing cancer in a subject, the method comprising administering an antigen binding site of the invention to the subject. In this regard, an antigen binding site can be used to prevent a relapse of a condition, and this is considered preventing the condition.

Exemplary cancers include prostate cancer. It will be understood that the antibodies having affinity for PSMA will be useful for this purpose.

Other exemplary cancers include renal cancer. It will be understood that the antibodies of the invention having affinity for CAIX will be useful for this purpose.

The present invention also provides an in vivo method of diagnosing, monitoring or prognosing a disease, disorder or infection in a subject comprising:
(a) administering to a subject an effective amount of the antibody herein described, said antibody specifically binding to an antigen associated with a disease, disorder or infection;
(b) allowing the antibody to concentrate at sites in said subject where said antigen is found; and
(c) detecting said antibody;
whereby detection of said antibody above a background or standard level indicates that the subject has said disease disorder or infection.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1: Average half-life, and Tukey's multiple comparison of half-life, of the antibodies of the invention. Error bars represent standard error of the mean. A: ChGmAb=chimeric girentuximab. HuGmAb=humanised girentuximab; HuGmAb-FcRn=humanised girentuximab with amino acid substitutions H310A and 435Ω. HuGmAb-FcRg=humanised girentuximab with amino acid substitutions S228P, 235E, H310A and 435Q. B. J591 IgG=control HuJ591 antibody for binding PSMA. ANT4044-K-DOTA=antibody ANT4044 lysine conjugated to DOTA. ANT4044-A2-K-DOTA—antibody ANT4044-A2 lysine conjugated to DOTA. ANT4044-FcRn-K-DOTA=antibody ANT4044 with amino acid substitutions in the FcRn-binding region, lysine conjugated to DOTA. ANT4044-FcRg-K-DOTA=antibody ANT4044 with amino acid substitutions in the FcRn and Fc gamma receptor binding regions, lysine conjugated to DOTA.

FIG. 2: Average area under the curve (AUC) and clearance (CL) for selected antibodies of the invention. J591 IgG=control HuJ591 antibody for binding PSMA. ANT4044-K-DOTA=antibody ANT4044 lysine conjugated to DOTA. ANT4044-A2-K-DOTA-antibody ANT4044-A2 lysine conjugated to DOTA. ANT4044-FcRn-K-DOTA=antibody ANT4044 with amino acid substitutions in the FcRn-binding region, lysine conjugated to DOTA. ANT4044-FcRg-K-DOTA=antibody ANT4044 with amino acid substitutions in the FcRn and Fc gamma receptor binding regions, lysine conjugated to DOTA.

FIG. 3: Biodistribution of antibodies at 8 hrs as determined by ROI analysis of PET images.

FIG. 4: Biodistribution of antibodies at 24 hrs as determined by ROI analysis of PET images.

FIG. 5: Biodistribution of all antibodies at 48 hrs as determined by ex vivo gamma count.

FIG. 6: Biodistribution of antibodies at 48 hrs as determined by ROI analysis of PET images.

FIG. 7: Blood concentration of antibodies out to 5 days post-injection.

FIG. 8: Tumour accumulation of antibodies as determined by imaging (8 h, 24 h, 48 hr)

FIG. 9: The ratio of antibody in tumour to blood. The tumour:blood ratio (in vivo tumour:tail blood) for each of the antibodies was determined for the 8 hr, 24 hr and 48 hr time points. The ratio is significantly higher for JN006 and JN007 at all time points compared with antibodies JN005 and hJ591.

FIG. 10: The ratio of antibody in tumour to blood. The tumour:blood ratio (ex vivo:x vivo) for each of the antibodies was determined for at 48 hr and 120 hr time points. The ratio is significantly higher for JN006 and JN007 at all time points compared with antibodies JN005 and hJ591.

FIG. 11: In vivo imaging and in vivo distribution of an exemplary antibody of the invention. SPECT imaging in LNCap xenograft mice that received an anti-PSMA, FcRn-K-DOTA-Lu-modified antibody of the invention.

FIG. 12: Blood pharmacokinetics of an exemplary antibody of the invention. Levels of radioactivity measured in blood of mice following administration of an anti-PSMA, FcRn-K-DOTA-Lu-modified antibody of the invention.

FIG. 13: Efficacy study in LNCap-bearing xenograft mice that were treated with an exemplary antibody of the invention. Treatment with an anti-PSMA, K-DOTA-Lu FcRn-modified antibody of the invention significantly suppressed tumour growth as evidenced by no change in tumour volume on day 14 as compared to day 0.

In the control (PBS) group, there was an overall increase in tumour volume with tumours becoming significant larger at 9, 12 and 14 days when compared to the corresponding time in FcRn-K-DOTA-Lu treated group.

FIG. 14: Tumour:blood ratios in LNCap-bearing xenograft mice following administration of an exemplary antibody of the invention. Tumour:blood ratios are shown for mice that received treatment with an anti-PSMA, K-DOTA-Lu antibody of the invention that is modified to reduce FcRn-binding (HuX592R-DOTA-Lu177). Control mice were administered an anti-PSMA, K-DOTA-Lu antibody (HuJ591-DOTA-Lu177). The ratio is higher for the mice that received the FcRn modified antibody compared to mice that received the unmodified antibody, particularly at 24 hour and 48 hour time points.

FIG. 15: Time activity curves of uptake of ⁸⁹Zr-DFOsq-FcRn-GmAb and ⁸⁹Zr-DFOsq-cGmAb-CHO into tumour, and ratios of tumor to tissues. PET images were analysed and results are shown as mean±SEM, n=3, **** P<0.0001.

FIG. 16: Ex-vivo biodistribution analysis. Mice were euthanised at 24, 48 and 144 hr p.i. with ⁸⁹Zr-DFOsq-FcRn-GmAb (top panel) and ⁸⁹Zr-DFOsq-cGmAb-CHO (lower panel). Tissues were harvested, weighed and counted on a gamma counter. Tracer uptake is expressed as percent injected dose/gram tissue. Data represents mean±SEM, n=3, except 24 hr cGmAb-CHO where n=2.

FIG. 17: ¹⁷⁷Lu-labelled HuX592R and HuJ591 biodistribution in healthy male Balb/c nude mice measured by ex vivo gamma counting. A shows HuX592R biodistribution assessed at 24, 48 and 72 hours post administration while B compares biodistribution of HuX592R with HuJ591 at 72 hours post administration.

FIG. 18: Regression of LNCaP xenograft tumours in each therapeutic cohort following administration of HuX592R (FcRn-K-DOTA-Lu), or no treatment control.

FIG. 19: Plot of cohort survival over the day period of the study following administration of TLX592 (FcRn-K-DOTA-Lu), TLX591 (K-DOTA-Lu antibody, also referred to herein as HuJ591-DOTA-Lu177) or no treatment control.

FIG. 20: Therapeutic efficacy of an exemplary ²²⁵-Ac-DOTA- or ¹⁷⁷-Lu-DOTA-labelled humanised anti-CAIX binding antibody of the invention. Change in mean tumour size (mm³) over time following administration of ²²⁵-Ac-DOTA-hG250 or ¹⁷⁷-Lu-DOTA-hG250, compared to no-treatment control.

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

Further aspects of the present invention and further embodiments of the aspects described in the preceding paragraphs will become apparent from the following description, given by way of example and with reference to the accompanying drawings.

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

The present invention is directed in part to the identification of a new approach for reducing the toxicity of radioimmunoconjugates for use in radioimmunotherapy. In particular, the method of the present invention reduces toxicity without significantly impacting on the therapeutic potential of the radioimmunoconjugates.

As previously outlined above, radiation damage to healthy tissues and cells is a major problem associated with radioimmunotherapy. The toxicity severely limits the radiation dosage of RAIT and reduces the effectiveness of tumor treatment.

However, the present inventors have developed antibodies for use in radioimmunotherapy that have reduced serum half-life compared to wild-type antibodies by virtue of modifications to the constant heavy chain of the antibody, reducing the affinity of the antibodies for the neonatal Fc receptor (FcRn). The antibodies developed by the present inventors therefore have significant benefits for use in various immunotherapies.

While modification of the FcRn-binding domain of therapeutic antibodies has previously been reported, such modifications have been developed with the aim of increasing FcRn affinity, for example to increase serum half-life of therapeutic antibodies and prolong residence of the therapeutic antibodies in the circulation. In contrast to the approaches of the prior art, the present invention is aimed at reducing serum half-life of a therapeutic agent.

Surprisingly, the inventors have found that despite reducing serum half-life (and increasing the rate of clearance from the systemic circulation following administration), the antibodies of the present invention have a similar capacity as unmodified antibodies to be delivered to and to accumulate at the tumour site. These results are surprising in that they show that tumour loading for modified and unmodified antibodies is not statistically different, indicating that the approach adopted by the present inventors, while significantly reducing serum half-life and thereby toxicity, does not negatively impact on the ability of the antibodies to bind to their target epitopes, nor on the capacity of the antibodies to be delivered to the target sites.

More importantly, the inventors have shown that the modified antibodies of the present invention remain resident at the tumour site, despite having increased clearance. The work of the present inventors therefore indicates that modification of the FcRn or the FcRn and Fc gamma receptor binding domains of radiolabelled antibodies has significant utility in reducing the amount of radioisotope in the circulation, without impacting on the therapeutic potential of the antibody with respect to its capacity to accumulate in the tumour. This has numerous benefits, including reducing a number of toxic effects which would otherwise result from longer-term residence of radioisotope in the circulation (including haematological toxicity, as a result of bone marrow irradiation and absorption into bone). Moreover, considering that the dose-limiting toxicity for many RAIT therapies thus far has been haematologic toxicities as a direct result of this extended blood circulation and irradiation of the bone marrow, this inventions affords the likelihood of acceptable dosing at higher levels; thus leading to a more effective therapy. absorption into bone and bone marrow irradiation).

Unexpectedly, the inventors have also found that the amino acid modifications to the constant heavy chain, while abrogating and FcRn binding does not impact on the ability of the antibodies to bind to Protein G and some Protein A purification resins. Thus the antibodies of the present invention, having reduced serum half-life compared to other immunotherapeutics, can be produced using the same existing/standardised production platform developed for regular antibodies. This is a key advantage over several of the many other engineered antibody formats e.g. minibodies, diabodies etc, which are cumbersome to produce and less stable than IgG molecules. Also, as a molecule format that is ‘native’ to the body, full-length antibodies also tend to have reduced likelihood of an immunogenic response than engineered antibodies or antibody fragments.

A further advantage of the antibodies of the present invention is their particular suitability for application in the field of theranostics. More specifically, as described above, the reduced serum half-life of the antibodies, when coupled to a radioactive isotope, makes the antibodies particularly useful for therapy, since the antibodies are able to deliver a suitable amount of radioactive agent to the tumour, while being rapidly cleared from the circulation. In addition, the reduced serum half-life of the antibodies makes them particularly suitable for use in diagnostic methods, where rapid clearing of the radioisotope coupled to the antibody and selected for imaging is desirable. The use of the antibody in the first instance as a diagnostic thereby informs the use and dosing of the therapeutic form of the antibody (i.e., when the antibody is coupled to a radioisotope that is suitable for therapy). Thus the antibodies of the invention find utility when coupled to different radioisotopes and subsequently employed as a “theranostic pair”.

In addition to the above, the present inventors herein describe novel humanised antibodies for binding to carbonic anhydrase (CAIX) and antibodies for binding to prostate specific membrane antigen (PSMA). These antibodies are described herein both for use as unmodified antibodies, or as antibodies with modifications to the FcRn and Fc gamma receptor binding portions of the heavy constant chains.

General Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter. Thus, as used herein, the singular forms “a”, “an” and “the” include plural aspects, and vice versa, unless the context clearly dictates otherwise. For example, reference to “a” includes a single as well as two or more; reference to “an” includes a single as well as two or more; reference to “the” includes a single as well as two or more and so forth.

Those skilled in the art will appreciate that the present invention is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

One skilled in the art will recognize many methods and materials similar or equivalent to those described herein, which could be used in the practice of the present invention. The present invention is in no way limited to the methods and materials described.

All of the patents and publications referred to herein are incorporated by reference in their entirety.

The present invention is not to be limited in scope by the specific examples described herein, which are intended for the purpose of exemplification only. Functionally-equivalent products, compositions and methods are clearly within the scope of the present invention.

Any example or embodiment of the present invention herein shall be taken to apply mutatis mutandis to any other example or embodiment of the invention unless specifically stated otherwise.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The description and definitions of variable regions and parts thereof, immunoglobulins, antibodies and fragments thereof herein may be further clarified by the discussion in Kabat Sequences of Proteins of Immunological Interest, National Institutes of Health, Bethesda, Md., 1987 and 1991, Bork et al., J Mol. Biol. 242, 309-320, 1994, Chothia and Lesk J. Mol Biol. 196:901-917, 1987, Chothia et al. Nature 342, 877-883, 1989 and/or or Al-Lazikani et al., J Mol Biol 273, 927-948, 1997.

The term “and/or”, e.g., “X and/or Y” shall be understood to mean either “X and Y” or “X or Y” and shall be taken to provide explicit support for both meanings or for either meaning.

As used herein the term “derived from” shall be taken to indicate that a specified integer may be obtained from a particular source albeit not necessarily directly from that source.

### Selected Definitions

As used herein, tumour:blood ratio refers to the ratio of the amount of an antibody (or radiolabelled antibody) to the amount of the same antibody in the blood of an individual. The skilled person will be familiar with standard techniques for calculating tumour:blood ratios. For example, ex vivo activity concentrations of radioisotope (or labelled antibody) are measured and expressed as percent of the decay-corrected injected activity per gram of tissue (or blood) and approximated as percentage injected dose/g (% ID/g). The tumour to blood ratio is then calculated as the activity detected in tumour relative to the activity detected in blood.

As used herein, the term ‘theranostic’ refers to the ability of compounds/materials to be used for diagnosis as well as for therapy. The term “theranostics reagent” relates to any reagent which is both suitable for detection, diagnostic and/or the treatment of a disease or condition of a patient. The aim of theranostic compounds/materials is to overcome undesirable differences in biodistribution and selectivity, which can exist between distinct diagnostic and therapeutic agents. With a theranostic pair, the theranostic compound containing the imaging radionuclide is first administered to the patient in order to identify the disease or to locate the affected area in the body. Once identified/located, the disease can be treated by administering the theranostic compound containing the therapeutic radionuclide in a target specific way as the biodistribution of the imaging and therapy radionuclides are the same.

In the context of the present invention, the antibodies of the invention are particularly useful for inclusion in theranostic pairs, for example, where the antibody is conjugated to a radioisotope for imaging or diagnostic purposes, and the same antibody is conjugated with a different radioisotope or a cytotoxic agent which is suitable for therapy. The antigen binding site of the antibody directs or targets the diagnostic radioisotope to the site of the tumour to facilitate diagnosis (including tumour distribution, tumour size, tumour density), while the same antigen binding site of the antibody directs the radioisotope to the tumour for therapy.

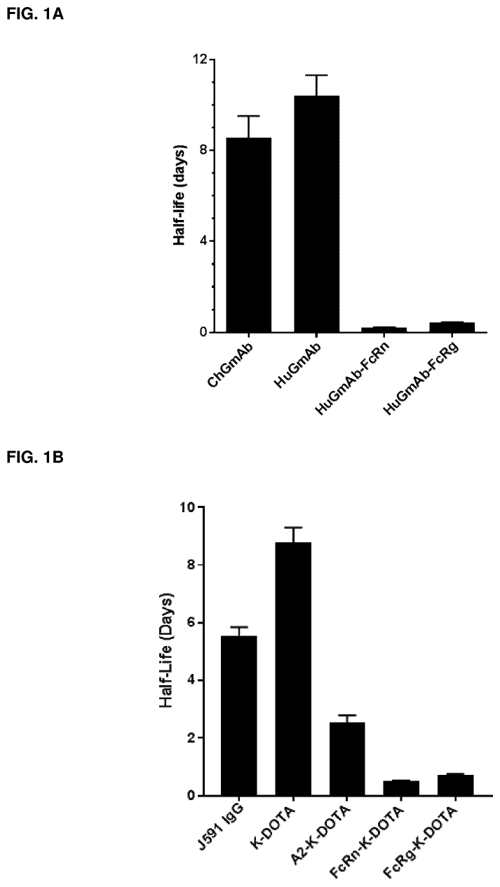

The term “Fc region”, sometimes referred to as “Fc” or “Fc domain”, as used herein refers the portion of an IgG molecule that correlates to a crystallizable fragment obtained by papain digestion of an IgG molecule. The Fc region consists of the C-terminal half of the two heavy chains of an IgG molecule that are linked by disulfide bonds. It has no antigen binding activity but contains the carbohydrate moiety and the binding sites for complement and Fc receptors, including the FcRn receptor. The Fc region contains the entire second constant domain CH2 (residues 231-340 of human IgG1, according to the EU Index numbering system, also defined as residues 244 to 360 in the Kabat system) and the third constant domain CH3 (residues 341-447 EU Index/361-478 Kabat) (e.g., see SEQ ID NO 1 of WO2015175874 or FIG. 1C for the sequence of CH2 and SEQ ID NO:2; FIG. 1D for the sequence of CH3, incorporated herein by reference; see also http://www.imgt.org/IMGTScientificChart/Numbering/Hu_IGHGnber.html #refs for a comparison of the numbering conventions used for various residues in the Fc region of immunoglobulins).

As used herein, the “EU index” or “EU numbering scheme” refers to the numbering of the EU antibody (Edelman et al., 1969, Proc Natl Acad Sci USA 63:78-85, hereby entirely incorporated by reference.) As used herein, the “Kabat system” refers to the Kabat Sequences of Proteins of Immunological Interest, National Institutes of Health, Bethesda, Md., 1987 and 1991. The skilled person will be able to readily determine whether a given amino acid sequence is numbered according to either EU or Kabat systems.

The term “isolated protein” or “isolated polypeptide” is a protein or polypeptide that by virtue of its origin or source of derivation is not associated with naturally-associated components that accompany it in its native state; is substantially free of other proteins from the same source. A protein may be rendered substantially free of naturally associated components or substantially purified by isolation, using protein purification techniques known in the art. By “substantially purified” is meant the protein is substantially free of contaminating agents, e.g., at least about 70% or 75% or 80% or 85% or 90% or 95% or 96% or 97% or 98% or 99% free of contaminating agents.

The term “recombinant” shall be understood to mean the product of artificial genetic recombination. Accordingly, in the context of a recombinant protein comprising an antibody antigen binding domain, this term does not encompass an antibody naturally-occurring within a subject's body that is the product of natural recombination that occurs during B cell maturation. However, if such an antibody is isolated, it is to be considered an isolated protein comprising an antibody antigen binding domain. Similarly, if nucleic acid encoding the protein is isolated and expressed using recombinant means, the resulting protein is a recombinant protein comprising an antibody antigen binding domain. A recombinant protein also encompasses a protein expressed by artificial recombinant means when it is within a cell, tissue or subject, e.g., in which it is expressed.

The term “protein” shall be taken to include a single polypeptide chain, i.e., a series of contiguous amino acids linked by peptide bonds or a series of polypeptide chains covalently or non-covalently linked to one another (i.e., a polypeptide complex). For example, the series of polypeptide chains can be covalently linked using a suitable chemical or a disulphide bond. Examples of non-covalent bonds include hydrogen bonds, ionic bonds, Van der Waals forces, and hydrophobic interactions.

The term “polypeptide” or “polypeptide chain” will be understood from the foregoing paragraph to mean a series of contiguous amino acids linked by peptide bonds.

As used herein, the term “antigen binding site” is used interchangeably with “antigen binding domain” and shall be taken to mean a region of an antibody that is capable of specifically binding to an antigen, i.e., a VH or a VL or an Fv comprising both a VH and a VL. The antigen binding domain need not be in the context of an entire antibody, e.g., it can be in isolation (e.g., a domain antibody) or in another form, e.g., as described herein, such as a scFv.

For the purposes for the present disclosure, the term “antibody” includes a protein capable of specifically binding to one or a few closely related antigens by virtue of an antigen binding domain contained within a Fv. This term includes four chain antibodies (e.g., two light chains and two heavy chains), recombinant or modified antibodies (e.g., chimeric antibodies, humanized antibodies, human antibodies, CDR-grafted antibodies, primatized antibodies, de-immunized antibodies, synhumanized antibodies, half-antibodies, bispecific antibodies). An antibody generally comprises constant domains, which can be arranged into a constant region or constant fragment or fragment crystallizable (Fc). Exemplary forms of antibodies comprise a four-chain structure as their basic unit. Full-length antibodies comprise two heavy chains (˜50 to 70 kD) covalently linked and two light chains (˜23 kDa each). A light chain generally comprises a variable region (if present) and a constant domain and in mammals is either a K light chain or a λ light chain. A heavy chain generally comprises a variable region and one or two constant domain(s) linked by a hinge region to additional constant domain(s). Heavy chains of mammals are of one of the following types α, δ, ε, γ, or μ. Each light chain is also covalently linked to one of the heavy chains. For example, the two heavy chains and the heavy and light chains are held together by inter-chain disulfide bonds and by non-covalent interactions. The number of inter-chain disulfide bonds can vary among different types of antibodies. Each chain has an N-terminal variable region (VH or VL wherein each are ˜110 amino acids in length) and one or more constant domains at the C-terminus. The constant domain of the light chain (CL which is ˜110 amino acids in length) is aligned with and disulfide bonded to the first constant domain of the heavy chain (CH1 which is 330 to 440 amino acids in length). The light chain variable region is aligned with the variable region of the heavy chain. The antibody heavy chain can comprise 2 or more additional CH domains (such as, CH2, CH3 and the like) and can comprise a hinge region between the CH1 and CH2 constant domains. Antibodies can be of any type (e.g., IgG, IgE, IgM, IgD, IgA, and IgY), class (e.g., IgG1, IgG2, IgG3, IgG4, IgA1 and IgA2) or subclass. In one example, the antibody is a murine (mouse or rat) antibody or a primate (such as, human) antibody. In one example the antibody heavy chain is missing a C-terminal lysine residue. In one example, the antibody is humanized, synhumanized, chimeric, CDR-grafted or deimmunized.

The terms “full-length antibody”, “intact antibody” or “whole antibody” are used interchangeably to refer to an antibody in its substantially intact form, as opposed to an antigen binding fragment of an antibody. Specifically, whole antibodies include those with heavy and light chains including an Fc region. The constant domains may be wild-type sequence constant domains (e.g., human wild-type sequence constant domains) or amino acid sequence variants thereof.

As used herein, “variable region” refers to the portions of the light and/or heavy chains of an antibody as defined herein that is capable of specifically binding to an antigen and, includes amino acid sequences of complementarity determining regions (CDRs); i.e., CDR1, CDR2, and CDR3, and framework regions (FRs). For example, the variable region comprises three or four FRs (e.g., FR1, FR2, FR3 and optionally FR4) together with three CDRs. VH refers to the variable region of the heavy chain. VL refers to the variable region of the light chain.

As used herein, the term “complementarity determining regions” (syn. CDRs; i.e., CDR1, CDR2, and CDR3) refers to the amino acid residues of an antibody variable region the presence of which are major contributors to specific antigen binding. Each variable region domain (VH or VL) typically has three CDRs identified as CDR1, CDR2 and CDR3. The CDRs of VH are also referred to herein as CDR H1, CDR H2 and CDR H3, respectively, wherein CDR H1 corresponds to CDR 1 of VH, CDR H2 corresponds to CDR 2 of VH and CDR H3 corresponds to CDR 3 of VH. Likewise, the CDRs of VL are referred to herein as CDR L1, CDR L2 and CDR L3, respectively, wherein CDR L1 corresponds to CDR 1 of VL, CDR L2 corresponds to CDR 2 of VL and CDR L3 corresponds to CDR 3 of VL. In one example, the amino acid positions assigned to CDRs and FRs are defined according to Kabat Sequences of Proteins of Immunological Interest, National Institutes of Health, Bethesda, Md., 1987 and 1991 (also referred to herein as “the Kabat numbering system”). In another example, the amino acid positions assigned to CDRs and FRs are defined according to the Enhanced Chothia Numbering Scheme (http://www.bioinfo.org.uk/mdex.html). The present invention is not limited to FRs and CDRs as defined by the Kabat numbering system, but includes all numbering systems, including the canonical numbering system or of Chothia and Lesk J. Mol. Biol. 196: 901-917, 1987; Chothia et al., Nature 342: 877-883, 1989; and/or Al-Lazikani et al., J. Mol. Biol. 273: 927-948, 1997; the numbering system of Honnegher and Plükthun J. Mol. Biol. 309: 657-670, 2001; or the IMGT system discussed in Giudicelli et al., Nucleic Acids Res. 25: 206-211 1997. In one example, the CDRs are defined according to the Kabat numbering system. Optionally, heavy chain CDR2 according to the Kabat numbering system does not comprise the five C-terminal amino acids listed herein or any one or more of those amino acids are substituted with another naturally-occurring amino acid. In this regard, Padlan et al., FASEB J., 9: 133-139, 1995 established that the five C-terminal amino acids of heavy chain CDR2 are not generally involved in antigen binding.

“Framework regions” (FRs) are those variable region residues other than the CDR residues. The FRs of VH are also referred to herein as FR H1, FR H2, FR H3 and FR H4, respectively, wherein FR H1 corresponds to FR 1 of VH, FR H2 corresponds to FR 2 of VH, FR H3 corresponds to FR 3 of VH and FR H4 corresponds to FR 4 of VH. Likewise, the FRs of VL are referred to herein as FR L1, FR L2, FR L3 and FR L4, respectively, wherein FR L1 corresponds to FR 1 of VL, FR L2 corresponds to FR 2 of VL, FR L3 corresponds to FR 3 of VL and FR L4 corresponds to FR 4 of VL.

As used herein, the term “Fv” shall be taken to mean any protein, whether comprised of multiple polypeptides or a single polypeptide, in which a VL and a VH associate and form a complex having an antigen binding domain, i.e., capable of specifically binding to an antigen. The VH and the VL which form the antigen binding domain can be in a single polypeptide chain or in different polypeptide chains. Furthermore, an Fv of the invention (as well as any protein of the invention) may have multiple antigen binding domains which may or may not bind the same antigen. This term shall be understood to encompass fragments directly derived from an antibody as well as proteins corresponding to such a fragment produced using recombinant means. In some examples, the VH is not linked to a heavy chain constant domain (CH) 1 and/or the VL is not linked to a light chain constant domain (CL). Exemplary Fv containing polypeptides or proteins include a Fab fragment, a Fab′ fragment, a F(ab′) fragment, a scFv, a diabody, a triabody, a tetrabody or higher order complex, or any of the foregoing linked to a constant region or domain thereof, e.g., CH2 or CH3 domain, e.g., a minibody. A “Fab fragment” consists of a monovalent antigen-binding fragment of an immunoglobulin, and can be produced by digestion of a whole antibody with the enzyme papain, to yield a fragment consisting of an intact light chain and a portion of a heavy chain or can be produced using recombinant means. A “Fab′ fragment” of an antibody can be obtained by treating a whole antibody with pepsin, followed by reduction, to yield a molecule consisting of an intact light chain and a portion of a heavy chain comprising a VH and a single constant domain. Two Fab′ fragments are obtained per antibody treated in this manner. A Fab′ fragment can also be produced by recombinant means. A “F(ab′)2 fragment” of an antibody consists of a dimer of two Fab′ fragments held together by two disulfide bonds, and is obtained by treating a whole antibody molecule with the enzyme pepsin, without subsequent reduction. A “Fab2” fragment is a recombinant fragment comprising two Fab fragments linked using, for example a leucine zipper or a CH3 domain. A “single chain Fv” or “scFv” is a recombinant molecule containing the variable region fragment (Fv) of an antibody in which the variable region of the light chain and the variable region of the heavy chain are covalently linked by a suitable, flexible polypeptide linker.

As used herein, the term “binds” in reference to the interaction of an antigen binding site or an antigen binding domain thereof with an antigen means that the interaction is dependent upon the presence of a particular structure (e.g., an antigenic determinant or epitope) on the antigen. For example, an antibody recognizes and binds to a specific protein structure rather than to proteins generally. If an antibody binds to epitope “A”, the presence of a molecule containing epitope “A” (or free, unlabelled “A”), in a reaction containing labeled “A” and the protein, will reduce the amount of labelled “A” bound to the antibody.

As used herein, the term “specifically binds” or “binds specifically” shall be taken to mean that an antigen binding site of the invention reacts or associates more frequently, more rapidly, with greater duration and/or with greater affinity with a particular antigen or cell expressing same than it does with alternative antigens or cells. For example, an antigen binding site binds to PSMA or CAIX with materially greater affinity (e.g., 1.5 fold or 2 fold or 5 fold or 10 fold or 20 fold or 40 fold or 60 fold or 80 fold to 100 fold or 150 fold or 200 fold) than it does to other antigens.

As used herein, the term “epitope” (syn. “antigenic determinant”) shall be understood to mean a region of a cell surface protein (such as PSMA or CAIX) to which an antigen binding site comprising an antigen binding domain of an antibody binds.

As used herein, the term “condition” refers to a disruption of or interference with normal function, and is not to be limited to any specific condition, and will include diseases or disorders.

As used herein, the terms “preventing”, “prevent” or “prevention” include administering an antigen binding site of the invention to thereby stop or hinder the development of at least one symptom of a condition. This term also encompasses treatment of a subject in remission to prevent or hinder relapse.

As used herein, the terms “treating”, “treat” or “treatment” include administering an antigen binding site described herein to thereby reduce or eliminate at least one symptom of a specified disease or condition.

As used herein, the term “subject” shall be taken to mean any animal including humans, for example a mammal. Exemplary subjects include but are not limited to humans and non-human primates. For example, the subject is a human.

### Modified Antibodies

The present invention relates in part to modifications to IgG antibodies, which include one or more amino acid substitutions to a region of the antibody which reduces or abolishes the affinity of the antibody for FcRn, thereby reducing the serum half-life of the antibodies.

It will be understood that in accordance with the present invention, any antibody for which reduced serum half-life is desired can be modified according to the methods described herein. Moreover, it will be understood that in preferred embodiments, the antibody that is modified to reduce serum half-life is an antibody that is useful in diagnostic or therapeutic applications, and more specifically in theranostic applications.

Examples of suitable antibodies for use in accordance with the methods of the present invention include, trastuzumab (Herceptin®), rituximab (Rituxan®), bevacizumab (Avastin®), dinutuximab (Unituxin®), panitumumab (Vectibix®), pembrolizumab (Keytruda®), nivolumab (Opdivo®), tositumomab (Bexxar®) ibritumomab (Zevalin®). However, it will also be understood that the present invention is not intended to be limited to the specific antibody, provided that the antibody would otherwise be susceptible to binding by FcRn.

The present invention also contemplates the use of antibody drug conjugates for targeting tumour antigens, wherein the conjugates include cytotoxic payload. Examples of such antibodies include gemtuzumab (Mylotarg®), brentuximab (Adcetris®), Inotuzumab (Besponsa®), glembatumumab (CDX-011), anetumab (BAY 94-9343), mirvetuximab (IMGN853) depatuxizumab (ABT-414), rovalpituzumab (Rova-T) and vadastuximab talirine (SGN-CD33A). Further examples are described in Lambert et al., 2017, Adv Ther (2017) 34:1015-1035, incorporated herein by reference.

In certain embodiments, the antibodies suitable for modification according the present invention, to reduce affinity for FcRn, are antibodies having one or more of the sequences as shown in Tables 1 and 2.

The present invention also provides an antigen binding site or a nucleic acid encoding same having at least 80% identity to a sequence disclosed herein. In one example, an antigen binding site or nucleic acid of the invention comprises sequence at least about 85% or 90% or 95% or 97% or 98% or 99% identical to a sequence disclosed herein.

Alternatively, or additionally, the antigen binding site comprises a CDR (e.g., three CDRs) at least about 80% or 85% or 90% or 95% or 97% or 98% or 99% identical to CDR(s) of a V_{H} or V_{L} as described herein according to any example.

In another example, a nucleic acid of the invention comprises a sequence at least about 80% or 85% or 90% or 95% or 97% or 98% or 99% identical to a sequence encoding an antigen binding site having a function as described herein according to any example. The present invention also encompasses nucleic acids encoding an antigen binding site of the invention, which differs from a sequence exemplified herein as a result of degeneracy of the genetic code.

The % identity of a nucleic acid or polypeptide is determined by GAP (Needleman and Wunsch. Mol. Biol. 48, 443-453, 1970) analysis (GCG program) with a gap creation penalty=5, and a gap extension penalty=0.3. The query sequence is at least 50 residues in length, and the GAP analysis aligns the two sequences over a region of at least 50 residues. For example, the query sequence is at least 100 residues in length and the GAP analysis aligns the two sequences over a region of at least 100 residues. For example, the two sequences are aligned over their entire length.

The present invention also contemplates a nucleic acid that hybridizes under stringent hybridization conditions to a nucleic acid encoding an antigen binding site described herein. A “moderate stringency” is defined herein as being a hybridization and/or washing carried out in 2×SSC buffer, 0.1% (w/v) SDS at a temperature in the range 45° C. to 65° C., or equivalent conditions. A “high stringency” is defined herein as being a hybridization and/or wash carried out in 0.1×SSC buffer, 0.1% (w/v) SDS, or lower salt concentration, and at a temperature of at least 65° C., or equivalent conditions. Reference herein to a particular level of stringency encompasses equivalent conditions using wash/hybridization solutions other than SSC known to those skilled in the art. For example, methods for calculating the temperature at which the strands of a double stranded nucleic acid will dissociate (also known as melting temperature, or Tm) are known in the art. A temperature that is similar to (e.g., within 5° C. or within 10° C.) or equal to the Tm of a nucleic acid is considered to be high stringency. Medium stringency is to be considered to be within 10° C. to 20° C. or 10° C. to 15° C. of the calculated Tm of the nucleic acid.

The present invention also contemplates mutant forms of an antigen binding site of the invention comprising one or more conservative amino acid substitutions compared to a sequence set forth herein. In some examples, the antigen binding site comprises 10 or fewer, e.g., 9 or 8 or 7 or 6 or 5 or 4 or 3 or 2 or 1 conservative amino acid substitutions. A “conservative amino acid substitution” is one in which the amino acid residue is replaced with an amino acid residue having a similar side chain and/or hydropathicity and/or hydrophilicity.

Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), #-branched side chains (e.g., threonine, valine, isoleucine) and aromatic side chains (e.g., tyrosine, phenylalanine, tryptophan, histidine). Hydropathic indices are described, for example in Kyte and Doolittle J. Mol. Biol., 157: 105-132, 1982 and hydrophilic indices are described in, e.g., U.S. Pat. No. 4,554,101.

The present invention also contemplates non-conservative amino acid changes. For example, of particular interest are substitutions of charged amino acids with another charged amino acid and with neutral or positively charged amino acids. In some examples, the antigen binding site comprises 10 or fewer, e.g., 9 or 8 or 7 or 6 or 5 or 4 or 3 or 2 or 1 non-conservative amino acid substitutions.

In one example, the mutation(s) occur within a FR of an antigen binding domain of an antigen binding site of the invention. In another example, the mutation(s) occur within a CDR of an antigen binding site of the invention.

Exemplary methods for producing mutant forms of an antigen binding site include:
mutagenesis of DNA (Thie et al., Methods Mol. Biol. 525: 309-322, 2009) or RNA (Kopsidas et al., Immunol. Lett. 107:163-168, 2006; Kopsidas et al. BMC Biotechnology, 7: 18, 2007; and WO1999/058661);
introducing a nucleic acid encoding the polypeptide into a mutator cell, e.g., XL-1 Red, XL-mutS and XL-mutS-Kanr bacterial cells (Stratagene);
DNA shuffling, e.g., as disclosed in Stemmer, Nature 370: 389-91, 1994; and
site directed mutagenesis, e.g., as described in Dieffenbach (ed) and Dveksler (ed) (In: PCR Primer: A Laboratory Manual, Cold Spring Harbor Laboratories, N Y, 1995).

Exemplary methods for determining biological activity of the mutant antigen binding sites of the invention will be apparent to the skilled artisan and/or described herein, e.g., antigen binding. For example, methods for determining antigen binding, competitive inhibition of binding, affinity, association, dissociation and therapeutic efficacy are described herein.

### Constant Regions

The present invention encompasses antigen binding sites and/or antibodies described herein comprising a constant region of an antibody. This includes antigen binding fragments of an antibody fused to an Fc.

Sequences of constant regions useful for producing the proteins of the present invention may be obtained from a number of different sources. In some examples, the constant region or portion thereof of the protein is derived from a human antibody. The constant region or portion thereof may be derived from any antibody class, including IgM, IgG, IgD, IgA and IgE, and any antibody isotype, including IgG1, IgG2, IgG3 and IgG4. In one example, the constant region is human isotype IgG4 or a stabilized IgG4 constant region.

### Preferred Modifications

The present invention specifically contemplates modifications to an antibody or antigen binding site comprising an Fc region or constant region.

The neonatal Fc-receptor (FcRn) is important for the metabolic fate of antibodies of the IgG class in vivo. The FcRn functions to salvage IgG from the lysosomal degradation pathway, resulting in reduced clearance and increased half-life. It is a heterodimeric protein consisting of two polypeptides: a 50 kDa class I major histocompatibility complex-like protein (a-FcRn) and a 15 kDa p2-microglobulin (β2ηI). FcRn binds with high affinity to the CH2-CH3 portion of the Fc-region of an antibody of the class IgG. The interaction between an antibody of the class IgG and the FcRn is pH dependent and occurs in a 1:2 stoichiometry, i.e. one IgG antibody molecule can interact with two FcRn molecules via its two heavy chain Fc-region polypeptides (see e.g. Huber, A. H., et al, J. Mol. Biol. 230 (1993) 1077-1083).

Thus, an IgG's in vitro FcRn binding properties/characteristics are indicative of its in vivo pharmacokinetic properties in the blood circulation. In the interaction between the FcRn and the Fc-region of an antibody of the IgG class different amino acid residues of the heavy chain CH2- and CH3-domain are participating.

Different mutations that influence the FcRn binding and therewith the half-live in the blood circulation are known. Fc-region residues critical to the mouse Fc-region-mouse FcRn interaction have been identified by site-directed mutagenesis (see e.g. Dall'Acqua, W. F., et al. J. Immunol 169 (2002) 5171-5180). Residues Ile253, His310, His433, Asn434 and His435 (numbering according to EU index numbering system) are involved in the interaction (Medesan, C, et al., Eur. J. Immunol. 26 (1996) 2533-2536; Firan, M., et al, Int. Immunol. 13 (2001) 993-1002; Kim, J. K., et al, Eur. J. Immunol. 24 (1994) 542-548). (Using the Kabat system, the relevant residues are Ile266, His329, His464, Asn465 and His466). Residues Ile253, His310, and His435 were found to be critical for the interaction of human Fc-region with murine FcRn (Kim, J. K., et al, Eur. J. Immunol. 29 (1999) 2819-2885).

More specifically, the antibody may comprise one or more amino acid substitutions that decrease the half-life of the protein. For example, the antibody comprises a Fc region comprising one or more amino acid substitutions that decrease the affinity of the Fc region for the neonatal Fc region (FcRn).

The present invention also provides for an antibody having a constant region substantially identical to a naturally occurring class IgG antibody constant region wherein at least one amino acid residue selected from the group consisting of residues His310, His435, and Ile253 is different from that present in the naturally occurring class IgG antibody, thereby altering FcRn binding affinity and/or serum half-life of said antibody relative to the naturally occurring antibody. In preferred embodiments, the naturally occurring class IgG antibody comprises a heavy chain constant region of a human IgG1, IgG2, IgG2M3, IgG3 or IgG4 molecule.

Also in preferred embodiments, amino acid residue 310 or residue 435 from the heavy chain constant region of the antibody having a constant region substantially identical to the naturally occurring class IgG antibody is any amino acid that is not histidine and which reduces the affinity of the constant region for FcRn. For example, the amino acid at residue 310 or 435 may be alanine, glutamic acid, aspartic acid, leucine, isoleucine, arginine, proline, glutamine, methionine, serine, threonine, lysine, asparagine, phenylalanine, tyrosine, tryptophan, cysteine, valine or glycine.

Preferably, the residue at position 310 is selected from alanine, or glutamic acid or glutamine; or amino acid residue 435 from the heavy chain constant region is selected from arginine, glutamine or alanine. In other preferred embodiments, the antibody having a constant region substantially identical to a naturally occurring class IgG antibody has an alanine residue at position 310 and glutamine residue at position 435.

In a preferred embodiment of the present invention, the binding affinity for FcRn and/or the serum half-life of the modified antibody is decreased by at least about 30%, 50%, 80%, 2-fold, 3-fold, 4-fold, 5-fold, 10-fold, 15-fold, 20-fold, 25-fold, 30-fold, 40-fold, 50-fold, 60-fold, 70-fold, 80-fold, 90-fold, or 100-fold. In a preferred embodiment of the present invention, the binding affinity for FcRn and/or the serum half-life of said modified antibody is reduced by at least about 20%, 30%, 40%, 50%, 60%, 70%, 80%, 85%, 90%, 95%, 97%, 98%, or 99%.

In addition, the antibodies of the present invention may comprise one or more mutations which modify the affinity of the antibodies for any one or more Fc gamma receptors.

In preferred embodiments of the invention, the Fc region of the constant region retains the ability to induce effector functions. In one example, the Fc region of the constant region contains one or more amino acid substitutions that modulate effector function, including increasing effector function compared to a wild-type IgG.

In one example, the Fc region of the constant region has a reduced ability to induce effector function, e.g., compared to a native or wild-type human IgG1 or IgG3 Fc region. In one example, the effector function is antibody-dependent cell-mediated cytotoxicity (ADCC) and/or antibody-dependent cell-mediated phagocytosis (ADCP) and/or complement-dependent cytotoxicity (CDC). Methods for assessing the level of effector function of an Fc region containing protein are known in the art and/or described herein.

In one example, the amino acid substitution that modifies that ability of the antibody to induce effector function is an amino acid substitution at residue Ile253 from the heavy chain constant region. In one example, the substitution is to any amino acid selected from be alanine, glutamic acid, aspartic acid, leucine, isoleucine, arginine, proline, glutamine, methionine, serine, threonine, lysine, asparagine, phenylalanine, tyrosine, tryptophan, cysteine, valine or glycine, wherein the substitution reduces the ability of the antibody to induce effector function. In preferred embodiments, the substitution from Ile at residue 253 is to arginine, proline, or aspartate, more preferably alanine.

In one example, the Fc region is an IgG4 Fc region (i.e., from an IgG4 constant region), e.g., a human IgG4 Fc region. Sequences of suitable IgG4 Fc regions will be apparent to the skilled person and/or available in publicly available databases (e.g., available from National Center for Biotechnology Information).

In one example, the constant region is a stabilized IgG4 constant region. The term “stabilized IgG4 constant region” will be understood to mean an IgG4 constant region that has been modified to reduce Fab arm exchange or the propensity to undergo Fab arm exchange or formation of a half-antibody or a propensity to form a half antibody. “Fab arm exchange” refers to a type of protein modification for human IgG4, in which an IgG4 heavy chain and attached light chain (half-molecule) is swapped for a heavy-light chain pair from another IgG4 molecule. Thus, IgG4 molecules may acquire two distinct Fab arms recognizing two distinct antigens (resulting in bispecific molecules). Fab arm exchange occurs naturally in vivo and can be induced in vitro by purified blood cells or reducing agents such as reduced glutathione. A “half antibody” forms when an IgG4 antibody dissociates to form two molecules each containing a single heavy chain and a single light chain.

In one example, a stabilized IgG4 constant region comprises a proline at position 241 of the hinge region according to the system of Kabat (Kabat et al., Sequences of Proteins of Immunological Interest Washington DC United States Department of Health and Human Services, 1987 and/or 1991). This position corresponds to position 228 of the hinge region according to the EU numbering system. In human IgG4, this residue is generally a serine. Following substitution of the serine for proline, the IgG4 hinge region comprises a sequence CPPC. In this regard, the skilled person will be aware that the “hinge region” is a proline-rich portion of an antibody heavy chain constant region that links the Fc and Fab regions that confers mobility on the two Fab arms of an antibody. The hinge region includes cysteine residues which are involved in inter-heavy chain disulfide bonds. It is generally defined as stretching from Glu226 to Pro243 of human IgG1 according to the numbering system of Kabat (or Glu216 to Pro230 using the EU index). Hinge regions of other IgG isotypes may be aligned with the IgG1 sequence by placing the first and last cysteine residues forming inter-heavy chain disulphide (S-S) bonds in the same positions (see for example WO2010/080538).

Additional examples of stabilized IgG4 antibodies are antibodies in which arginine at position 409 in a heavy chain constant region of human IgG4 (according to the EU numbering system) is substituted with lysine, threonine, methionine, or leucine (e.g., as described in WO2006/033386). The Fc region of the constant region may additionally or alternatively comprise a residue selected from the group consisting of: alanine, valine, glycine, isoleucine and leucine at the position corresponding to 405 (according to the EU numbering system). Optionally, the hinge region comprises a proline at position 241 (i.e., a CPPC sequence) (as described above).

In another example, the Fc region is a region modified to have reduced effector function, i.e., a “non-immunostimulatory Fc region”. For example, the Fc region is an IgG1 Fc region comprising a substitution at one or more positions selected from the group consisting of 268, 309, 330 and 331. In another example, the Fc region is an IgG1 Fc region comprising one or more of the following changes E233P, L234V, L235A and deletion of G236 and/or one or more of the following changes A327G, A330S and P331 S (Armour et al., Eur J Immunol. 29:2613-2624, 1999; Shields et al., J Biol Chem. 276(9):6591-604, 2001). Additional examples of non-immunostimulatory Fc regions are described, for example, in Dall'Acqua et al., J Immunol. 177: 1129-1138 2006; and/or Hezareh J Virol; 75: 12161-12168, 2001).

In another example, the Fc region is a chimeric Fc region, e.g., comprising at least one C_{H}² domain from an IgG4 antibody and at least one C_{H}³ domain from an IgG1 antibody, wherein the Fc region comprises a substitution at one or more amino acid positions selected from the group consisting of 240, 262, 264, 266, 297, 299, 307, 309, 323, 399, 409 and 427 (EU numbering) (e.g., as described in WO2010/085682). Exemplary substitutions include 240F, 262L, 264T, 266F, 297Q, 299A, 299K, 307P, 309K, 309M, 309P, 323F, 399S, and 427F.

### Antibody Production

Preferably, an antigen binding site described herein according to any example is recombinant.

In the case of a recombinant protein, nucleic acid encoding same can be cloned into expression constructs or vectors, which are then transfected into host cells, such as E. coli cells, yeast cells, insect cells, or mammalian cells, such as simian COS cells, Chinese Hamster Ovary (CHO) cells, human embryonic kidney (HEK) cells, or myeloma cells that do not otherwise produce the protein. Exemplary cells used for expressing a protein are CHO cells, myeloma cells or HEK cells. Molecular cloning techniques to achieve these ends are known in the art and described, for example in Ausubel et al., (editors), Current Protocols in Molecular Biology, Greene Pub. Associates and Wiley-Interscience (1988, including all updates until present) or Sambrook et al., Molecular Cloning: A Laboratory Manual, Cold Spring Harbor Laboratory Press (1989). A wide variety of cloning and in vitro amplification methods are suitable for the construction of recombinant nucleic acids. Methods of producing recombinant antibodies are also known in the art, see, e.g., U.S. Pat. No. 4,816,567 or U.S. Pat. No. 5,530,101.

Following isolation, the nucleic acid is inserted operably linked to a promoter in an expression construct or expression vector for further cloning (amplification of the DNA) or for expression in a cell-free system or in cells.

As used herein, the term “promoter” is to be taken in its broadest context and includes the transcriptional regulatory sequences of a genomic gene, including the TATA box or initiator element, which is required for accurate transcription initiation, with or without additional regulatory elements (e.g., upstream activating sequences, transcription factor binding sites, enhancers and silencers) that alter expression of a nucleic acid, e.g., in response to a developmental and/or external stimulus, or in a tissue specific manner. In the present context, the term “promoter” is also used to describe a recombinant, synthetic or fusion nucleic acid, or derivative which confers, activates or enhances the expression of a nucleic acid to which it is operably linked. Exemplary promoters can contain additional copies of one or more specific regulatory elements to further enhance expression and/or alter the spatial expression and/or temporal expression of said nucleic acid.

As used herein, the term “operably linked to” means positioning a promoter relative to a nucleic acid such that expression of the nucleic acid is controlled by the promoter.

Many vectors for expression in cells are available. The vector components generally include, but are not limited to, one or more of the following: a signal sequence, a sequence encoding a protein (e.g., derived from the information provided herein), an enhancer element, a promoter, and a transcription termination sequence. The skilled artisan will be aware of suitable sequences for expression of a protein. Exemplary signal sequences include prokaryotic secretion signals (e.g., pelB, alkaline phosphatase, penicillinase, Ipp, or heat-stable enterotoxin II), yeast secretion signals (e.g., invertase leader, α factor leader, or acid phosphatase leader) or mammalian secretion signals (e.g., herpes simplex gD signal).

Exemplary promoters active in mammalian cells include cytomegalovirus immediate early promoter (CMV-IE), human elongation factor 1-α promoter (EF1), small nuclear RNA promoters (U1a and U1b), α-myosin heavy chain promoter, Simian virus 40 promoter (SV40), Rous sarcoma virus promoter (RSV), Adenovirus major late promoter, β-actin promoter; hybrid regulatory element comprising a CMV enhancer/β-actin promoter or an immunoglobulin promoter or active fragment thereof. Examples of useful mammalian host cell lines are monkey kidney CV1 line transformed by SV40 (COS-7, ATCC CRL 1651); human embryonic kidney line (293 or 293 cells subcloned for growth in suspension culture; baby hamster kidney cells (BHK, ATCC CCL 10); or Chinese hamster ovary cells (CHO).

Typical promoters suitable for expression in yeast cells such as for example a yeast cell selected from the group comprising Pichia pastoris, Saccharomyces cerevisiae and S. pombe, include, but are not limited to, the ADH1 promoter, the GAL1 promoter, the GAL4 promoter, the CUP1 promoter, the PHO5 promoter, the not promoter, the RPR1 promoter, or the TEF1 promoter.

Means for introducing the isolated nucleic acid or expression construct comprising same into a cell for expression are known to those skilled in the art. The technique used for a given cell depends on the known successful techniques. Means for introducing recombinant DNA into cells include microinjection, transfection mediated by DEAE-dextran, transfection mediated by liposomes such as by using lipofectamine (Gibco, MD, USA) and/or cellfectin (Gibco, MD, USA), PEG-mediated DNA uptake, electroporation and microparticle bombardment such as by using DNA-coated tungsten or gold particles (Agracetus Inc., WI, USA) amongst others.

The host cells used to produce the protein may be cultured in a variety of media, depending on the cell type used. Commercially available media such as Ham's FI0 (Sigma), Minimal Essential Medium ((MEM), (Sigma), RPMI-1640 (Sigma), and Dulbecco's Modified Eagle's Medium ((DMEM), Sigma) are suitable for culturing mammalian cells. Media for culturing other cell types discussed herein are known in the art.

### Isolation of Proteins

Methods for isolating a protein are known in the art and/or described herein.

Where an antigen binding site is secreted into culture medium, supernatants from such expression systems can be first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants. Alternatively, or additionally, supernatants can be filtered and/or separated from cells expressing the protein, e.g., using continuous centrifugation.

The antigen binding site prepared from the cells can be purified using, for example, ion exchange, hydroxyapatite chromatography, hydrophobic interaction chromatography, gel electrophoresis, dialysis, affinity chromatography (e.g., protein A affinity chromatography or protein G chromatography), or any combination of the foregoing. These methods are known in the art and described, for example in WO99/57134 or Ed Harlow and David Lane (editors) Antibodies: A Laboratory Manual, Cold Spring Harbor Laboratory, (1988).

The skilled artisan will also be aware that a protein can be modified to include a tag to facilitate purification or detection, e.g., a poly-histidine tag, e.g., a hexa-histidine tag, or an influenza virus hemagglutinin (HA) tag, or a Simian Virus 5 (V5) tag, or a FLAG tag, or a glutathione S-transferase (GST) tag. The resulting protein is then purified using methods known in the art, such as, affinity purification. For example, a protein comprising a hexa-his tag is purified by contacting a sample comprising the protein with nickel-nitrilotriacetic acid (Ni-NTA) that specifically binds a hexa-his tag immobilized on a solid or semi-solid support, washing the sample to remove unbound protein, and subsequently eluting the bound protein. Alternatively, or in addition a ligand or antibody that binds to a tag is used in an affinity purification method.

### Linking of Radioisotopes to Antibodies

In any embodiment of the invention, the antibodies herein described may be directly or indirectly linked to a diagnostic or therapeutic agent, Preferably, the diagnostic or therapeutic agent is a radioisotope.

Examples of suitable isotopes include: actinium-225 (²²⁵Ac), astatine-211 (²¹¹At), bismuth-212 and bismuth-213 (²¹²Bi, ²¹³Bi), copper-64 and copper-67 (⁶⁴Cu, ⁶⁷Cu), gallium-67 and gallium-68 (⁶⁷Ga and ⁶⁸Ga), indium-111 (¹¹¹In), iodine-123, -124, -125 or -131 (¹²³I, ¹²⁴I, ¹²⁵I, ¹³¹I) (123), lead-212 (²¹²Pb), lutetium-77 (¹⁷⁷Lu), radium-223 (²²³Ra), samarium-153 (¹⁵³Sm), scandium-44 and scandium-47 (44Sc, ⁴⁷Sc), strontium-90 (⁹⁰Sr), technetium-99 (^{99m}Tc), yttrium-86 and yttrium-90 (⁸⁶Y, ⁹⁰Y), zirconium-89 (⁸⁹Zr). The skilled person will be familiar with which radioisotopes are preferable for use as diagnostic agents and which are preferably for use as therapeutics.

It will be understood that the radioisotopes may be conjugated to the antibodies of the invention directly (via a chelating agent or prosthetic group or linker) or indirectly via binding to single or multiple amino acid residues in the antibody (e.g. halogenation of tyrosine residues).

In alternative embodiments, chelating agents or linkers may be used in order to conjugate the radioisotope to the antibody. In one example, the antibodies can be conjugated to a chelating moiety, selected from the group consisting of: TMT (6,6″-bis[N,N″,N′″-tetra(carboxymethyl)aminomethyl)-4′-(3-amino-4-methoxyphenyl)-2,2′:6′,2″-terpyridine), DOTA (1,4,7,10-tetraazacyclododecane-NN′,N″(N′″-tetraacetic acid, also known as tetraxetan), TCMC (the tetra-primary amide of DOTA), DO3A (1,4,7,10-Tetraazacyclododecane-1,4,7-tris(acetic acid)-10-(2-thioethyl)acetamide), CB-DO2A (4,10-bis(carboxymethyl)-1,4,7,10-tetraazabicyclo[5.5.2]tetradecan), NOTA (1,4,7-triazacyclononane-triacetic acid) Diamsar (3,6,10,13,16,19-hexaazabicyclo[6.6.6]eicosane-1,8-diamine), DTPA (Pentetic acid or diethylenetriaminepentaacetic acid), CHX-A″-DTPA ([(R)-2-Amino-3-(4-isothiocyanatophenyl)propyl]-trans-(S,S)-cyclohexane-1,2-diamine-pentaacetic acid), TETA (1,4,8,11-tetraazacyclotetradecane-1,4,8), 11-tetraacetic acid, Te2A (4,11-bis(carboxymethyl)-1,4,8,11-tetraazabicyclo[6.6.2]hexadecane), HBED, DFO (Desferrioxamine), DFOsq (DFO-squaramide) and HOPO (3,4,3-(LI-1,2-HOPO) or other chelating agent as described herein.

Chelators with radiometals and other halogenated radioisotopes may be bound to the antibodies of the invention via one or more amino acid residues or reactive moieties in the antibody, including but not limited to one or more lysine residues, tyrosine residues or thiol moieties.

In another example, the modified antibody is conjugated to a bifunctional linker, for example, bromoacetyl, thiols, succinimide ester, TFP ester, a maleimide, or using any amine or thiol-modifying chemistry known in the art.

The skilled person will be familiar with standard methods for conjugating chelating agents to antibodies and derivatives or fragments thereof. In addition, the skilled person will be familiar with approaches for selecting a relevant chelating agent for pairing with a radiometal, for example as described in Chem. Soc. Rev., 2014, 43, 260, incorporated herein by reference.

### Assaying Activity of an Antigen Binding Site

### Binding to PSMA or CAIX

It will be apparent to the skilled artisan from the disclosure herein that the preferred antigen binding sites of the present invention bind to PSMA or to CAIX. Methods for assessing binding to a protein are known in the art, e.g., as described in Scopes (In: Protein purification: principles and practice, Third Edition, Springer Verlag, 1994). Such a method generally involves immobilizing the antigen binding site and contacting it with labeled antigen. Following washing to remove non-specific bound protein, the amount of label and, as a consequence, bound antigen is detected. Of course, the antigen binding site can be labeled and the antigen immobilized. Panning-type assays can also be used. Alternatively, or additionally, surface plasmon resonance assays can be used.

### Therapeutic, Diagnostic and Theranostic Methods

The antibodies of the present invention are useful for treating a number of conditions requiring treatment by radioimmunotherapy. Typically, such conditions include cancer.

Exemplary cancers include cystic and solid tumors, bone and soft tissue tumors, including tumors in anal tissue, bile duct, bladder, blood cells, bowel, brain, breast, carcinoid, cervix, eye, esophagus, head and neck, kidney, larynx, leukemia, liver, lung, lymph nodes, lymphoma, melanoma, mesothelioma, myeloma, ovary, pancreas, penis, prostate, skin (e.g. squamous cell carcinoma), sarcomas, stomach, testes, thyroid, vagina, vulva. Soft tissue tumors include Benign schwannoma Monosomy, Desmoid tumor, lipo-blastoma, lipoma, uterine leiomyoma, clear cell sarcoma, dermatofibrosarcoma, Ewing sarcoma, extraskeletal myxoid chondrosarcoma, liposarcooma myxoid, Alveolar rhabdomyosarcoma and synovial sarcoma. Specific bone tumors include nonossifying fibroma, unicameral bone cyst, enchon-droma, aneurismal bone cyst, osteoblastoma, chondroblastoma, chondromyxofibroma, ossifying fibroma and adamantinoma, Giant cell tumor, fibrous dysplasia, Ewing's sarcoma eosinophilic granuloma, osteosarcoma, chondroma, chondrosarcoma, malignant fibrous histiocytoma and metastatic carcinoma. Leukemias include acute lymphoblastic, acute myeloblastic, chronic lymphocytic and chronic myeloid.

Other examples include breast tumors, colorectal tumors, adenocarcinomas, mesothelioma, bladder tumors, prostate tumors, germ cell tumor, hepatoma/cholongio, carcinoma, neuroendocrine tumors, pituitary neoplasm, small round cell tumor, squamous cell cancer, melanoma, atypical fibroxanthoma, seminomas, nonseminomas, stromal leydig cell tumors, Sertoli cell tumors, skin tumors, kidney tumors, testicular tumors, brain tumors, ovarian tumors, stomach tumors, oral tumors, bladder tumors, bone tumors, cervical tumors, esophageal tumors, laryngeal tumors, liver tumors, lung tumors, vaginal tumors and Wilm's tumor.

Preferably, the antigen binding sites of the present invention are useful for treating cancer that are characterised by the presence of PSMA or of CAIX. For example, the antibodies that bind to PSMA are useful for treating cancers characterised by increased expression of PSMA, including prostate cancer. The antibodies that bind to CAIX are useful for treating cancers characterised by increased expression of CAIX, including renal cell carcinoma.

The skilled person will be familiar with methods for selecting suitable diagnostic agents for use with the antibodies of the invention, including radioisotopes for use in radioimaging for diagnosing conditions described herein. Further, the skilled person will be familiar with imaging techniques for use in conjunction with the diagnostic reagents described herein.

As used herein, a theranostic method is a method for the in vitro and/or in vivo visualization, identification and/or detection of tumour cells and/or metastases as well as a method of treatment of cancer.

In one embodiment, the present invention includes a theranostic method that comprises:
(1) administering a diagnostically-effective amount an antibody of the present invention to a patient or subject, wherein the antibody comprises at least one diagnostically useful label, and,
(2) administering a therapeutically-effective amount of an antibody of the present invention to a patient or subject in need thereof,
wherein the antibody comprises a tumour therapeutic(s) (e.g, radioisotope, toxin(s), drug(s)).

Preferably, step 1 and step 2 are conducted sequentially and the antibody in step 1 and step 2 are the same.

### Antibody Binding Domain Containing Proteins

### Single-Domain Antibodies

In some examples, an antigen binding site or protein of the invention is or comprises a single-domain antibody (which is used interchangeably with the term “domain antibody” or “dAb”). A single-domain antibody is a single polypeptide chain comprising all or a portion of the heavy chain variable region of an antibody. In certain examples, a single-domain antibody is a human single-domain antibody (Domantis, Inc., Waltham, MA; see, e.g., U.S. Pat. No. 6,248,516).

### Diabodies, Triabodies, Tetrabodies

In some examples, a protein of the invention is or comprises a diabody, triabody, tetrabody or higher order protein complex such as those described in WO98/044001 and/or WO94/007921.

For example, a diabody is a protein comprising two associated polypeptide chains, each polypeptide chain comprising the structure V_{L}-X-V_{H} or V_{H}-X-V_{L}, wherein V_{L} is an antibody light chain variable region, V_{H} is an antibody heavy chain variable region, X is a linker comprising insufficient residues to permit the V_{H} and V_{L} in a single polypeptide chain to associate (or form an Fv) or is absent, and wherein the V_{H} of one polypeptide chain binds to a V_{L} of the other polypeptide chain to form an antigen binding domain, i.e., to form a Fv molecule capable of specifically binding to one or more antigens. The V_{L} and V_{H} can be the same in each polypeptide chain or the V_{L} and V_{H} can be different in each polypeptide chain so as to form a bispecific diabody (i.e., comprising two Fvs having different specificity).

Single Chain Fv (scFv)

The skilled artisan will be aware that scFvs comprise V_{H} and V_{L} regions in a single polypeptide chain and a polypeptide linker between the V_{H} and V_{L} which enables the scFv to form the desired structure for antigen binding (i.e., for the V_{H} and V_{L} of the single polypeptide chain to associate with one another to form a Fv). For example, the linker comprises in excess of 12 amino acid residues with (Gly₄Ser)₃ being one of the more favored linkers for a scFv.

The present invention also contemplates a disulfide stabilized Fv (or diFv or dsFv), in which a single cysteine residue is introduced into a FR of V_{H} and a FR of VL and the cysteine residues linked by a disulfide bond to yield a stable Fv.

Alternatively, or in addition, the present invention encompasses a dimeric scFv, i.e., a protein comprising two scFv molecules linked by a non-covalent or covalent linkage, e.g., by a leucine zipper domain (e.g., derived from Fos or Jun). Alternatively, two scFvs are linked by a peptide linker of sufficient length to permit both scFvs to form and to bind to an antigen, e.g., as described in US20060263367.

### Heavy Chain Antibodies

Heavy chain antibodies differ structurally from many other forms of antibodies, in so far as they comprise a heavy chain, but do not comprise a light chain. Accordingly, these antibodies are also referred to as “heavy chain only antibodies”. Heavy chain antibodies are found in, for example, camelids and cartilaginous fish (also called IgNAR).

The variable regions present in naturally occurring heavy chain antibodies are generally referred to as “VHH domains” in camelid antibodies and V-NAR in IgNAR, in order to distinguish them from the heavy chain variable regions that are present in conventional 4-chain antibodies (which are referred to as “V_{H} domains”) and from the light chain variable regions that are present in conventional 4-chain antibodies (which are referred to as “VL domains”).

A general description of heavy chain antibodies from camelids and the variable regions thereof and methods for their production and/or isolation and/or use is found inter alia in the following references WO94/04678, WO97/49805 and WO 97/49805.

A general description of heavy chain antibodies from cartilaginous fish and the variable regions thereof and methods for their production and/or isolation and/or use is found inter alia in WO2005/118629.

### Other Antibodies and Proteins Comprising Antigen Binding Domains Thereof.

The present invention also contemplates other antibodies and proteins comprising antigen-binding domains thereof, such as:
(i) “key and hole” bispecific proteins as described in U.S. Pat. No. 5,731,168;
(ii) heteroconjugate proteins, e.g., as described in U.S. Pat. No. 4,676,980;
(iii) heteroconjugate proteins produced using a chemical cross-linker, e.g., as described in U.S. Pat. No. 4,676,980; and
   (iv) Fab3 (e.g., as described in EP19930302894).

### Compositions

In some examples, an antigen binding site as described herein can be administered orally, parenterally, by inhalation spray, adsorption, absorption, topically, rectally, nasally, bucally, vaginally, intraventricularly, via an implanted reservoir in dosage formulations containing conventional non-toxic pharmaceutically-acceptable carriers, or by any other convenient dosage form. The term “parenteral” as used herein includes subcutaneous, intravenous, intramuscular, intraperitoneal, intrathecal, intraventricular, intrasternal, and intracranial injection or infusion techniques.

Methods for preparing an antigen binding site into a suitable form for administration to a subject (e.g. a pharmaceutical composition) are known in the art and include, for example, methods as described in Remington's Pharmaceutical Sciences (18th ed., Mack Publishing Co., Easton, Pa., 1990) and U.S. Pharmacopeia: National Formulary (Mack Publishing Company, Easton, Pa., 1984).

The pharmaceutical compositions of this invention are particularly useful for parenteral administration, such as intravenous administration or administration into a body cavity or lumen of an organ or joint. The compositions for administration will commonly comprise a solution of an antigen binding site dissolved in a pharmaceutically acceptable carrier, for example an aqueous carrier. A variety of aqueous carriers can be used, e.g., buffered saline and the like. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of an antigen binding site of the present invention in these formulations can vary widely, and will be selected primarily based on fluid volumes, viscosities, body weight and the like in accordance with the particular mode of administration selected and the patient's needs. Exemplary carriers include water, saline, Ringer's solution, dextrose solution, and 5% human serum albumin. Nonaqueous vehicles such as mixed oils and ethyl oleate may also be used. Liposomes may also be used as carriers. The vehicles may contain minor amounts of additives that enhance isotonicity and chemical stability, e.g., buffers and preservatives.

### Dosages and Timing of Administration

Suitable dosages of an antigen binding site of the present invention will vary depending on the specific an antigen binding site, the condition to be treated and/or the subject being treated. It is within the ability of a skilled physician to determine a suitable dosage, e.g., by commencing with a sub-optimal dosage and incrementally modifying the dosage to determine an optimal or useful dosage. Alternatively, to determine an appropriate dosage for treatment/prophylaxis, data from the cell culture assays or animal studies are used, wherein a suitable dose is within a range of circulating concentrations that include the ED₅₀ of the active compound with little or no toxicity. The dosage may vary within this range depending upon the dosage form employed and the route of administration utilized. A therapeutically/prophylactically effective dose can be estimated initially from cell culture assays. A dose may be formulated in animal models to achieve a circulating plasma concentration range that includes the IC₅₀ (i.e., the concentration or amount of the compound which achieves a half-maximal inhibition of symptoms) as determined in cell culture. Such information can be used to more accurately determine useful doses in humans. Levels in plasma maybe measured, for example, by high performance liquid chromatography.

In some examples, a method of the present invention comprises administering a prophylactically or therapeutically effective amount of a protein described herein.

The term “therapeutically effective amount” is the quantity which, when administered to a subject in need of treatment, improves the prognosis and/or state of the subject and/or that reduces or inhibits one or more symptoms of a clinical condition described herein to a level that is below that observed and accepted as clinically diagnostic or clinically characteristic of that condition. The amount to be administered to a subject will depend on the particular characteristics of the condition to be treated, the type and stage of condition being treated, the mode of administration, and the characteristics of the subject, such as general health, other diseases, age, sex, genotype, and body weight. A person skilled in the art will be able to determine appropriate dosages depending on these and other factors. Accordingly, this term is not to be construed to limit the present invention to a specific quantity, e.g., weight or amount of protein(s), rather the present invention encompasses any amount of the antigen binding site(s) sufficient to achieve the stated result in a subject.

As used herein, the term “prophylactically effective amount” shall be taken to mean a sufficient quantity of a protein to prevent or inhibit or delay the onset of one or more detectable symptoms of a clinical condition. The skilled artisan will be aware that such an amount will vary depending on, for example, the specific antigen binding site(s) administered and/or the particular subject and/or the type or severity or level of condition and/or predisposition (genetic or otherwise) to the condition. Accordingly, this term is not to be construed to limit the present invention to a specific quantity, e.g., weight or amount of antigen binding site(s), rather the present invention encompasses any amount of the antigen binding site(s) sufficient to achieve the stated result in a subject.

### Kits

The present invention additionally comprises a kit comprising one or more of the following:
(i) an antibody of the invention or expression construct(s) encoding same;
(ii) a molecule of the invention;
(iii) a complex of the invention; or
(iii) a pharmaceutical composition of the invention.

In the case of a kit for detecting cancer, the kit can additionally comprise a detection means, e.g., linked to an antigen binding site of the invention.

In the case of a kit for therapeutic/prophylactic use, the kit can additionally comprise a pharmaceutically acceptable carrier.

Optionally a kit of the invention is packaged with instructions for use in a method described herein according to any example.

TABLE 1
Summary of amino acid and nucleotide sequences for PSMA-binding
antibodies of the invention

| | | SEQ | |
|---|---|---|---|
| | | ID | |
| Antibody ID | Region | NO: | Amino acid or nucleotide sequence |
| ANT4044 Variable | HCDR1 (protein) | 1 | EYTIH |
| Heavy chain | HCDR2 (protein) | 2 | NINPNNGGTTYNQKFED |
| | HCDR3 (protein) | 3 | GWNFDY |
| | VH (protein) | 4 | EVQLVQSGAEVKKPGASVKVSCKASGYTFTEYTIHW |
| | | | VRQAPGKGLEWIGNINPNNGGTTYNQKFEDRVTITV |
| | | | DKSTSTAYMELSSLRSEDTAVYYCAAGVVNFDYWGQ |
| | | | GTTVTVSS |
| | HCDR1 (DNA) | 5 | GAATACACCATCCAC |
| | HCDR2 (DNA) | 6 | AACATTAATCCTAACAATGGTGGTACTACCTACAAC |
| | | | CAGAAGTTCGAGGAC |
| | HCDR3 (DNA) | 7 | GGTTGGAACTTTGACTAC |
| | VH (DNA) | 8 | GAGGTCCAGCTGGTGCAGTCTGGAGCTGAGGTGA |
| | | | AGAAGCCTGGGGCCTCAGTGAAGGTCTCCTGCAA |
| | | | GGCTTCTGGATACACATTCACTGAATACACCATCC |
| | | | ACTGGGTGAGGCAGGCCCCTGGAAAGGGCCTTGA |
| | | | GTGGATTGGAAACATTAATCCTAACAATGGTGGTA |
| | | | CTACCTACAACCAGAAGTTCGAGGACAGAGTCACA |
| | | | ATCACTGTAGACAAGTCCACCAGCACAGCCTACAT |
| | | | GGAGCTCAGCAGCCTGAGATCTGAGGATACTGCA |
| | | | GTCTATTACTGTGCAGCTGGTTGGAACTTTGACTA |
| | | | CTGGGGCCAAGGCACCACGGTCACCGTCTCCTCA |
| | HFR1 (protein) | 9 | EVQLVQSGAEVKKPGASVKVSCKASGYTFT |
| | HFR2 (protein) | 10 | WVRQAPGKGLEWIG |
| | HFR3 (protein) | 11 | RVTITVDKSTSTAYMELSSLRSEDTAVYYCAA |
| | HFR4 (protein) | 12 | WGQGTTVTVSS |
| | HFR1 (DNA) | 12 | GAGGTCCAGCTGGTGCAGTCTGGAGCTGAGGTGA |
| | | | AGAAGCCTGGGGCCTCAGTGAAGGTCTCCTGCAA |
| | | | GGCTTCTGGATACACATTCACT |
| | HFR2 (DNA) | 14 | TGGGTGAGGCAGGCCCCTGGAAAGGGCCTTGAGT |
| | | | GGATTGGA |
| | HFR3 (DNA) | 15 | AGAGTCACAATCACTGTAGACAAGTCCACCAGCAC |
| | | | AGCCTACATGGAGCTCAGCAGCCTGAGATCTGAG |
| | | | GATACTGCAGTCTATTACTGTGCAGCT |
| | HFR4 (DNA) | 16 | TGGGGCCAAGGCACCACGGTCACCGTCTCCTCA |
| ANT4044-A2 | HCDR1 (protein) | 17 | EYTIH |
| Variable Heavy | HCDR2 (protein) | 18 | NINPNNGGTTYNQKFED |
| chain | HCDR3 (protein) | 19 | YWLFDY |
| | VH (protein) | 20 | EVQLVQSGAEVKKPGASVKVSCKASGYTFTEYTIHW |
| | | | VRQAPGKGLEWIGNINPNNGGTTYNQKFEDRVTITV |
| | | | DKSTSTAYMELSSLRSEDTAVYYCAAYWLFDYWGQ |
| | | | GTTVTVSS |
| | HCDR1 (DNA) | 21 | GAATACACCATCCAC |
| | HCDR2 (DNA) | 22 | AACATTAATCCTAACAATGGTGGTACTACCTACAAC |
| | | | CAGAAGTTCGAGGAC |
| | HCDR3 (DNA) | 23 | TACTGGCTGTTCGACTAC |
| | VH (DNA) | 24 | GAGGTCCAGCTGGTGCAGTCTGGAGCTGAGGTGA |
| | | | AGAAGCCTGGGGCCTCAGTGAAGGTCTCCTGCAA |
| | | | GGCTTCTGGATACACATTCACTGAATACACCATCC |
| | | | ACTGGGTGAGGCAGGCCCCTGGAAAGGGCCTTGA |
| | | | GTGGATTGGAAACATTAATCCTAACAATGGTGGTA |
| | | | CTACCTACAACCAGAAGTTCGAGGACAGAGTCACA |
| | | | ATCACTGTAGACAAGTCCACCAGCACAGCCTACAT |
| | | | GGAGCTCAGCAGCCTGAGATCTGAGGATACTGCA |
| | | | GTCTATTACTGTGCAGCTTACTGGCTGTTCGACTA |
| | | | CTGGGGCCAAGGCACCACGGTCACCGTCTCCTCA |
| | HFR1 (protein) | 25 | EVQLVQSGAEVKKPGASVKVSCKASGYTFT |
| | HFR2 (protein) | 26 | WVRQAPGKGLEWIG |
| | HFR3 (protein) | 27 | RVTITVDKSTSTAYMELSSLRSEDTAVYYCAA |
| | HFR4 (protein) | 28 | WGQGTTVTVSS |
| | HFR1 (DNA) | 29 | GAGGTCCAGCTGGTGCAGTCTGGAGCTGAGGTGA |
| | | | AGAAGCCTGGGGCCTCAGTGAAGGTCTCCTGCAA |
| | | | GGCTTCTGGATACACATTCACT |
| | HFR2 (DNA) | 30 | TGGGTGAGGCAGGCCCCTGGAAAGGGCCTTGAGT |
| | | | GGATTGGA |
| | HFR3 (DNA) | 31 | AGAGTCACAATCACTGTAGACAAGTCCACCAGCAC |
| | | | AGCCTACATGGAGCTCAGCAGCCTGAGATCTGAG |
| | | | GATACTGCAGTCTATTACTGTGCAGCT |
| | HFR4 (DNA) | 32 | TGGGGCCAAGGCACCACGGTCACCGTCTCCTCA |
| ANT4044/ | LCDR1 (protein) | 33 | KASQDVGTAVD |
| ANT4044-A2 | LCDR2 | 34 | WASTRHT |
| Variable Light | (protein) | | |
| chain | LCDR3 (protein) | 35 | QQYNSYPLT |
| | VL (protein) | 36 | DIQMTQSPSTLSASVGDRVTITCKASQDVGTAVDWY |
| | | | QQKPGQAPKLLIYWASTRHTGVPDRFSGSGSGTDFT |
| | | | LTISRLQPEDFAVYYCQQYNSYPLTFGQGTKVDIK |
| | LCDR1 (DNA) | 37 | AAGGCCAGTCAGGATGTGGGTACTGCTGTAGAC |
| | LCDR2 (DNA) | 38 | TGGGCATCCACCCGGCACACT |
| | LCDR3 (DNA) | 39 | CAGCAATATAACAGCTATCCTCTCACG |
| | VL (DNA) | 40 | GACATTCAGATGACCCAGTCTCCCAGCACCCTGTC |
| | | | CGCATCAGTAGGAGACAGGGTCACCATCACTTGCA |
| | | | AGGCCAGTCAGGATGTGGGTACTGCTGTAGACTG |
| | | | GTATCAACAGAAACCAGGGCAAGCTCCTAAACTAC |
| | | | TGATTTACTGGGCATCCACCCGGCACACTGGAGTC |
| | | | CCTGATCGCTTCAGCGGCAGTGGATCTGGGACAG |
| | | | ATTTCACTCTCACCATCAGCAGACTGCAGCCTGAA |
| | | | GACTTTGCAGTTTATTACTGTCAGCAATATAACAGC |
| | | | TATCCTCTCACGTTCGGCCAGGGGACCAAGGTGG |
| | | | ATATCAAA |
| | LFR1 | 41 | DIQMTQSPSTLSASVGDRVTITC |
| | (protein) | | |
| | LFR2 (protein) | 42 | WYQQKPGQAPKLLIY |
| | LFR3 (protein) | 43 | GVPDRFSGSGSGTDFTLTISRLQPEDFAVYYC |
| | LFR4 | 44 | FGQGTKVDIK |
| | (protein) | | |
| | LFR1 | 45 | GACATTCAGATGACCCAGTCTCCCAGCACCCTGTC |
| | (DNA) | | CGCATCAGTAGGAGACAGGGTCACCATCACTTGC |
| | LFR2(DNA) | 46 | TGGTATCAACAGAAACCAGGGCAAGCTCCTAAACT |
| | | | ACTGATTTAC |
| | LFR3 (DNA) | 47 | GGAGTCCCTGATCGCTTCAGCGGCAGTGGATCTG |
| | | | GGACAGATTTCACTCTCACCATCAGCAGACTGCAG |
| | | | CCTGAAGACTTTGCAGTTTATTACTGT |
| | LFR4 | 48 | TTCGGCCAGGGGACCAAGGTGGATATCAAA |
| | (DNA) | | |
| ANT4044/ANT4044- | IgG1 HC | 235 | ASTKGPSVFPLAPSSKSTSGGTAALGCLVKDYFPEP |
| A2_unmodified | (protein) | | VTVSWNSGALTSGVHTFPAVLQSSGLYSLSSVVTVP |
| human IgG1 | | | SSSLGTQTYICNVNHKPSNTKVDKRVEPKSCDKTHT |
| heavy chain | | | CPPCPAPELLGGPSVFLFPPKPKDTLMISRTPEVTCV |
| constant region | | | VVDVSHEDPEVKFNWYVDGVEVHNAKTKPREEQYN |
| | | | STYRVVSVLTVLHQDWLNGKEYKCKVSNKALPAPIEK |
| | | | TISKAKGQPREPQVYTLPPSREEMTKNQVSLTCLVK |
| | | | GFYPSDIAVEWESNGQPENNYKTTPPVLDSDGSFFL |
| | | | YSKLTVDKSRWQQGNVFSCSVMHEALHNHYTQKSL |
| | | | SLSPGK |
| ANT4044/ANT4044- | IgG1 H310A | 236 | ASTKGPSVFPLAPSSKSTSGGTAALGCLVKDYFPEP |
| A2 modified | H435Q HC | | VTVSWNSGALTSGVHTFPAVLQSSGLYSLSSVVTVP |
| human IgG1 | (protein) | | SSSLGTQTYICNVNHKPSNTKVDKRVEPKSCDKTHT |
| heavy chain | | | CPPCPAPELLGGPSVFLFPPKPKDTLMISRTPEVTCV |
| constant region | | | VVDVSHEDPEVKFNWYVDGVEVHNAKTKPREEQYN |
| | | | STYRVVSVLTVLAQDWLNGKEYKCKVSNKALPAPIEK |
| | | | TISKAKGQPREPQVYTLPPSREEMTKNQVSLTCLVK |
| | | | GFYPSDIAVEVVESNGQPENNYKTTPPVLDSDGSFFL |
| | | | YSKLTVDKSRWQQGNVFSCSVMHEALHNQYTQKSL |
| | | | SLSPGK |
| ANT4044/ANT4044- | IgG4 S228P | 237 | ASTKGPSVFPLAPCSRSTSESTAALGCLVKDYFPEPV |
| A2_modified | L235E H310A | | TVSWNSGALTSGVHTFPAVLQSSGLYSLSSVVTVPS |
| human IgG4 | H435Q HC | | SSLGTKTYTCNVDHKPSNTKVDKRVESKYGPPCPPC |
| constant chain | (protein) | | PAPEFEGGPSVFLFPPKPKDTLMISRTPEVTCVVVDV |
| region | | | SQEDPEVQFNWYVDGVEVHNAKTKPREEQFNSTYR |
| | | | VVSVLTVLAQDWLNGKEYKCKVSNKGLPSSIEKTISK |
| | | | AKGQPREPQVYTLPPSQEEMTKNQVSLTCLVKGFYP |
| | | | SDIAVEWESNGQPENNYKTTPPVLDSDGSFFLYSRL |
| | | | TVDKSRWQEGNVFSCSVMHEALHNQYTQKSLSLSL |
| | | | GK |
| ANT4044/ANT4044- | Human κ LC | 238 | RTVAAPSVFIFPPSDEQLKSGTASVVCLLNNFYPREA |
| A2 kappa light | constant | | KVQWKVDNALQSGNSQESVTEQDSKDSTYSLSSTLT |
| chain constant | region | | LSKADYEKHKVYACEVTHQGLSSPVTKSFNRGEC |
| region | | | |
| ANT4044 | FcRn-null, IgG1 | 239 | EVQLVQSGAEVKKPGASVKVSCKASGYTFTEYTIHW |
| RADmAb IgG1 | allotype G1m(3) | | VRQAPGKGLEWIGNINPNNGGTTYNQKFEDRVTITV |
| heavy chain | H310A H435Q | | DKSTSTAYMELSSLRSEDTAVYYCAAGWNFDYWGQ |
| (HuX592r) | | | GTTVTVSSASTKGPSVFPLAPSSKSTSGGTAALGCLV |
| | | | KDYFPEPVTVSWNSGALTSGVHTFPAVLQSSGLYSL |
| | | | SSVVTVPSSSLGTQTYICNVNHKPSNTKVDKRVEPKS |
| | | | CDKTHTCPPCPAPELLGGPSVFLFPPKPKDTLMISRT |
| | | | PEVTCVVVDVSHEDPEVKFNWYVDGVEVHNAKTKP |
| | | | REEQYNSTYRVVSVLTVLAQDWLNGKEYKCKVSNK |
| | | | ALPAPIEKTISKAKGQPREPQVYTLPPSREEMTKNQV |
| | | | SLTCLVKGFYPSDIAVEWESNGQPENNYKTTPPVLD |
| | | | SDGSFFLYSKLTVDKSRWQQGNVFSCSVMHEALHN |
| | | | QYTQKSLSLSPGK |
| ANT4044 | FcRn+FcRγ-null, | 240 | EVQLVQSGAEVKKPGASVKVSCKASGYTFTEYTIHW |
| RADmAb IgG4 | IgG4 S228P | | VRQAPGKGLEWIGNINPNNGGTTYNQKFEDRVTITV |
| heavy chain | L235E H310A | | DKSTSTAYMELSSLRSEDTAVYYCAAGWNFDYWGQ |
| | H435Q | | GTTVTVSSASTKGPSVFPLAPCSRSTSESTAALGCLV |
| | | | KDYFPEPVTVSVVNSGALTSGVHTFPAVLQSSGLYSL |
| | | | SSVVTVPSSSLGTKTYTCNVDHKPSNTKVDKRVESK |
| | | | YGPPCPPCPAPEFEGGPSVFLFPPKPKDTLMISRTPE |
| | | | VTCVVVDVSQEDPEVQFNWYVDGVEVHNAKTKPRE |
| | | | EQFNSTYRVVSVLTVLAQDWLNGKEYKCKVSNKGLP |
| | | | SSIEKTISKAKGQPREPQVYTLPPSQEEMTKNQVSLT |
| | | | CLVKGFYPSDIAVEWESNGQPENNYKTTPPVLDSDG |
| | | | SFFLYSRLTVDKSRWQEGNVFSCSVMHEALHNQYT |
| | | | QKSLSLSLGK |
| ANT4044-A2 | FcRn-null, IgG1 | 241 | EVQLVQSGAEVKKPGASVKVSCKASGYTFTEYTIHW |
| RADmAb IgG1 | H310A H435Q | | VRQAPGKGLEWIGNINPNNGGTTYNQKFEDRVTITV |
| heavy chain | | | DKSTSTAYMELSSLRSEDTAVYYCAAYWLFDYWGQ |
| | | | GTTVTVSSASTKGPSVFPLAPSSKSTSGGTAALGCLV |
| | | | KDYFPEPVTVSVVNSGALTSGVHTFPAVLQSSGLYSL |
| | | | SSVVTVPSSSLGTQTYICNVNHKPSNTKVDKRVEPKS |
| | | | CDKTHTCPPCPAPELLGGPSVFLFPPKPKDTLMISRT |
| | | | PEVTCVVVDVSHEDPEVKFNWYVDGVEVHNAKTKP |
| | | | REEQYNSTYRVVSVLTVLAQDWLNGKEYKCKVSNK |
| | | | ALPAPIEKTISKAKGQPREPQVYTLPPSREEMTKNQV |
| | | | SLTCLVKGFYPSDIAVEWESNGQPENNYKTTPPVLD |
| | | | SDGSFFLYSKLTVDKSRWQQGNVFSCSVMHEALHN |
| | | | QYTQKSLSLSPGK |
| ANT4044-A2 | FcRn+FcRγ-null | 242 | EVQLVQSGAEVKKPGASVKVSCKASGYTFTEYTIHW |
| RADmAb IgG4 | IgG4 S228P | | VRQAPGKGLEWIGNINPNNGGTTYNQKFEDRVTITV |
| heavy chain | L235E H310A | | DKSTSTAYMELSSLRSEDTAVYYCAAYVVLFDYWGQ |
| | H435Q | | GTTVTVSSASTKGPSVFPLAPCSRSTSESTAALGCLV |
| | | | KDYFPEPVTVSWNSGALTSGVHTFPAVLQSSGLYSL |
| | | | SSVVTVPSSSLGTKTYTCNVDHKPSNTKVDKRVESK |
| | | | YGPPCPPCPAPEFEGGPSVFLFPPKPKDTLMISRTPE |
| | | | VTCVVVDVSQEDPEVQFNWYVDGVEVHNAKTKPRE |
| | | | EQFNSTYRVVSVLTVLAQDWLNGKEYKCKVSNKGLP |
| | | | SSIEKTISKAKGQPREPQVYTLPPSQEEMTKNQVSLT |
| | | | CLVKGFYPSDIAVEWESNGQPENNYKTTPPVLDSDG |
| | | | SFFLYSRLTVDKSRWQEGNVFSCSVMHEALHNQYT |
| | | | QKSLSLSLGK |
| ANT4044- | Vk Light chain | 243 | DIQMTQSPSTLSASVGDRVTITCKASQDVGTAVDWY |
| ANT4044-A2 Vk | | | QQKPGQAPKLLIYWASTRHTGVPDRFSGSGSGTDFT |
| Light chain | | | LTISRLQPEDFAVYYCQQYNSYPLTFGQGTKVDIKRT |
| | | | VAAPSVFIFPPSDEQLKSGTASVVCLLNNFYPREAKV |
| | | | QWKVDNALQSGNSQESVTEQDSKDSTYSLSSTLTLS |
| | | | KADYEKHKVYACEVTHQGLSSPVTKSFNRGEC |

TABLE 2
Summary of amino acid and nucleotide sequences for CAIX-binding
antibodies of the invention

| | | SEQ | |
|---|---|---|---|
| | | ID | |
| Antibody ID | Region | NO: | Amino acid or nucleotide sequence |
| Girentuximab | HCDR1 (protein) | 49 | NYYMS |
| chimeric VHO | HCDR2 (protein) | 50 | AINSDGGITYYLDTVKG |
| Variable Heavy | HCDR3 (protein) | 51 | HRSGYFSMDY |
| chain | VH (protein) | 52 | DVKLVESGGGLVKLGGSLKLSCAASGFTFSNYYMSWV |
| | | | RQTPEKRLELVAAINSDGGITYYLDTVKGRFTISRDNAK |
| | | | NTLYLQMSSLKSEDTALFYCARHRSGYFSMDYWGQG |
| | | | TSVTVSS |
| | HCDR1 (DNA) | 53 | AACTATTACATGTCT |
| | HCDR2 (DNA) | 54 | GCCATTAATAGTGATGGTGGTATCACCTACTATCTAG |
| | | | ACACTGTGAAGGGC |
| | HCDR3 (DNA) | 55 | CACCGCTCGGGCTACTTTTCTATGGACTAC |
| | VH (DNA) | 56 | GACGTGAAGCTCGTGGAGTCTGGGGGAGGCTTAGT |
| | | | GAAGCTTGGAGGATCCCTGAAACTCTCCTGTGCAGC |
| | | | CTCTGGATTCACTTTCAGTAACTATTACATGTCTTGG |
| | | | GTTCGCCAGACTCCAGAGAAGAGGCTGGAGTTGGT |
| | | | CGCAGCCATTAATAGTGATGGTGGTATCACCTACTA |
| | | | TCTAGACACTGTGAAGGGCCGATTCACCATTTCAAG |
| | | | AGACAATGCCAAGAACACCCTGTACCTGCAAATGAG |
| | | | CAGTCTGAAGTCTGAGGACACAGCCTTGTTTTACTG |
| | | | TGCAAGACACCGCTCGGGCTACTTTTCTATGGACTA |
| | | | CTGGGGTCAAGGAACCTCAGTCACCGTCTCCTCA |
| | HFR1 (protein) | 57 | DVKLVESGGGLVKLGGSLKLSCAASGFTFS |
| | HFR2 (protein) | 58 | WVRQTPEKRLELVA |
| | HFR3 (protein) | 59 | RFTISRDNAKNTLYLQMSSLKSEDTALFYCAR |
| | HFR4 (protein) | 60 | WGQGTSVTVSS |
| | HFR1 (DNA) | 61 | GACGTGAAGCTCGTGGAGTCTGGGGGAGGCTTAGT |
| | | | GAAGCTTGGAGGATCCCTGAAACTCTCCTGTGCAGC |
| | | | CTCTGGATTCACTTTCAGT |
| | HFR2 (DNA) | 62 | TGGGTTCGCCAGACTCCAGAGAAGAGGCTGGAGTT |
| | | | GGTCGCA |
| | HFR3 (DNA) | 63 | CGATTCACCATTTCAAGAGACAATGCCAAGAACACC |
| | | | CTGTACCTGCAAATGAGCAGTCTGAAGTCTGAGGAC |
| | | | ACAGCCTTGTTTTACTGTGCAAGA |
| | HFR4 (DNA) | 64 | TGGGGTCAAGGAACCTCAGTCACCGTCTCCTCA |
| Girentuximab | HCDR1 (protein) | 65 | NYYMS |
| humanised VH1 | HCDR2 (protein) | 66 | AINSDGGITYYLDTVKG |
| Variable Heavy | HCDR3 (protein) | 67 | HRSGYFSMDY |
| chain | VH (protein) | 68 | DVKLVESGGGLVKPGGSLRLSCAASGFTFSNYYMSW |
| | | | VRQAPGKGLELVAAINSDGGITYYLDTVKGRFTISRDN |
| | | | AKNTLYLQMSSLKSEDTALYYCARHRSGYFSMDYWG |
| | | | QGTSVTVSS |
| | HCDR1 (DNA) | 69 | AACTACTACATGAGC |
| | HCDR2 (DNA) | 70 | GCCATTAACAGTGACGGTGGCATCACCTACTACCTG |
| | | | GACACCGTGAAGGGC |
| | HCDR3 (DNA) | 71 | CACAGGAGCGGCTACTTCTCTATGGACTAC |
| | VH (DNA) | 72 | GACGTGAAGCTGGTGGAGTCTGGGGGAGGCTTGGT |
| | | | CAAGCCTGGAGGATCCCTGAGACTCTCCTGTGCAG |
| | | | CCTCTGGATTCACCTTCAGTAACTACTACATGAGCT |
| | | | GGGTGCGCCAGGCTCCAGGGAAGGGGCTGGAGCT |
| | | | GGTTGCCGCCATTAACAGTGACGGTGGCATCACCTA |
| | | | CTACCTGGACACCGTGAAGGGCCGATTCACCATCTC |
| | | | CAGGGACAACGCCAAGAACACCCTGTATCTGCAAAT |
| | | | GAGCAGCCTGAAGAGCGAGGACACGGCCCTGTATT |
| | | | ACTGTGCGAGACACAGGAGCGGCTACTTCTCTATGG |
| | | | ACTACTGGGGCCAGGGCACCAGCGTCACTGTCTCC |
| | | | TCA |
| | HFR1 (protein) | 73 | DVKLVESGGGLVKPGGSLRLSCAASGFTFS |
| | HFR2 (protein) | 74 | WVRQAPGKGLELVA |
| | HFR3 (protein) | 75 | RFTISRDNAKNTLYLQMSSLKSEDTALYYCAR |
| | HFR4 (protein) | 76 | WGQGTSVTVSS |
| | HFR1 (DNA) | 77 | GACGTGAAGCTGGTGGAGTCTGGGGGAGGCTTGGT |
| | | | CAAGCCTGGAGGATCCCTGAGACTCTCCTGTGCAG |
| | | | CCTCTGGATTCACCTTCAGT |
| | HFR2 (DNA) | 78 | TGGGTGCGCCAGGCTCCAGGGAAGGGGCTGGAGC |
| | | | TGGTTGCC |
| | HFR3 (DNA) | 79 | CGATTCACCATCTCCAGGGACAACGCCAAGAACACC |
| | | | CTGTATCTGCAAATGAGCAGCCTGAAGAGCGAGGA |
| | | | CACGGCCCTGTATTACTGTGCGAGA |
| | HFR4 (DNA) | 80 | TGGGGCCAGGGCACCAGCGTCACTGTCTCCTCA |
| Girentuximab | HCDR1 (protein) | 81 | NYYMS |
| humanised VH3 | HCDR2 (protein) | 82 | AINSDGGITYYLDTVKG |
| Variable Heavy | HCDR3 (protein) | 83 | HRSGYFSMDY |
| chain | VH (protein) | 84 | DVKLVESGGGLVKPGGSLRLSCAASGFTFSNYYMSW |
| | | | VRQAPGKGLELVAAINSDGGITYYLDTVKGRFTISRDN |
| | | | AKNTLYLQMNSLRAEDTALYYCARHRSGYFSMDYWG |
| | | | QGTLVTVSS |
| | HCDR1 (DNA) | 85 | AACTACTACATGAGC |
| | HCDR2 (DNA) | 86 | CCATTAACAGTGACGGTGGCATCACCTACTACCTGG |
| | | | ACACCGTGAAGGGC |
| | HCDR3 (DNA) | 87 | CACAGGAGCGGCTACTTCTCTATGGACTAC |
| | VH (DNA) | 88 | GACGTGAAGCTGGTGGAGTCTGGGGGAGGCTTGGT |
| | | | CAAGCCTGGAGGATCCCTGAGACTCTCCTGTGCAG |
| | | | CCTCTGGATTCACCTTCAGTAACTACTACATGAGCT |
| | | | GGGTGCGCCAGGCTCCAGGGAAGGGGCTGGAGCT |
| | | | GGTTGCCGCCATTAACAGTGACGGTGGCATCACCTA |
| | | | CTACCTGGACACCGTGAAGGGCCGATTCACCATCTC |
| | | | CAGGGACAACGCCAAGAACACCCTGTATCTGCAAAT |
| | | | GAACAGCCTGAGGGCCGAGGACACGGCCCTGTATT |
| | | | ACTGTGCGAGACACAGGAGCGGCTACTTCTCTATGG |
| | | | ACTACTGGGGCCAGGGCACCCTGGTCACTGTCTCC |
| | | | TCA |
| | HFR1 (protein) | 89 | DVKLVESGGGLVKPGGSLRLSCAASGFTFS |
| | HFR2 (protein) | 90 | WVRQAPGKGLELVA |
| | HFR3 (protein) | 91 | RFTISRDNAKNTLYLQMNSLRAEDTALYYCAR |
| | HFR4 (protein) | 92 | WGQGTLVTVSS |
| | HFR1 (DNA) | 93 | GACGTGAAGCTGGTGGAGTCTGGGGGAGGCTTGGT |
| | | | CAAGCCTGGAGGATCCCTGAGACTCTCCTGTGCAG |
| | | | CCTCTGGATTCACCTTCAGT |
| | HFR2 (DNA) | 94 | TGGGTGCGCCAGGCTCCAGGGAAGGGGCTGGAGC |
| | | | TGGTTGCC |
| | HFR3 (DNA) | 95 | CGATTCACCATCTCCAGGGACAACGCCAAGAACACC |
| | | | CTGTATCTGCAAATGAACAGCCTGAGGGCCGAGGA |
| | | | CACGGCCCTGTATTACTGTGCGAGA |
| | HFR4 (DNA) | 96 | TGGGGCCAGGGCACCCTGGTCACTGTCTCCTCA |
| Girentuximab | HCDR1 (protein) | 97 | NYYMS |
| humanised VH4 | HCDR2 (protein) | 98 | AINSDGGITYYLDTVKG |
| Variable Heavy | HCDR3 (protein) | 99 | HRSGYFSMDY |
| chain | VH (protein) | 100 | EVQLVESGGGLVKPGGSLRLSCAASGFTFSNYYMSW |
| | | | VRQAPGKGLELVAAINSDGGITYYLDTVKGRFTISRDN |
| | | | AKNTLYLQMNSLRAEDTALYYCARHRSGYFSMDYWG |
| | | | QGTLVTVSS |
| | HCDR1 (DNA) | 101 | AACTACTACATGAGC |
| | HCDR2 (DNA) | 102 | GCCATTAACAGTGACGGTGGCATCACCTACTACCTG |
| | | | GACACCGTGAAGGGC |
| | HCDR3 (DNA) | 103 | CACAGGAGCGGCTACTTCTCTATGGACTAC |
| | VH (DNA) | 104 | GAGGTGCAGCTGGTGGAGTCTGGGGGAGGCTTGGT |
| | | | CAAGCCTGGAGGATCCCTGAGACTCTCCTGTGCAG |
| | | | CCTCTGGATTCACCTTCAGTAACTACTACATGAGCT |
| | | | GGGTGCGCCAGGCTCCAGGGAAGGGGCTGGAGCT |
| | | | GGTTGCCGCCATTAACAGTGACGGTGGCATCACCTA |
| | | | CTACCTGGACACCGTGAAGGGCCGATTCACCATCTC |
| | | | CAGGGACAACGCCAAGAACACCCTGTATCTGCAAAT |
| | | | GAACAGCCTGAGGGCCGAGGACACGGCCCTGTATT |
| | | | ACTGTGCGAGACACAGGAGCGGCTACTTCTCTATGG |
| | | | ACTACTGGGGCCAGGGCACCCTGGTCACTGTCTCC |
| | | | TCA |
| | HFR1 (protein) | 105 | EVQLVESGGGLVKPGGSLRLSCAASGFTFS |
| | HFR2 (protein) | 106 | WVRQAPGKGLELVA |
| | HFR3 (protein) | 107 | RFTISRDNAKNTLYLQMNSLRAEDTALYYCAR |
| | HFR4 (protein) | 108 | WGQGTLVTVSS |
| | HFR1 (DNA) | 109 | GAGGTGCAGCTGGTGGAGTCTGGGGGAGGCTTGGT |
| | | | CAAGCCTGGAGGATCCCTGAGACTCTCCTGTGCAG |
| | | | CCTCTGGATTCACCTTCAGT |
| | HFR2 (DNA) | 110 | TGGGTGCGCCAGGCTCCAGGGAAGGGGCTGGAGC |
| | | | TGGTTGCC |
| | HFR3 (DNA) | 111 | CGATTCACCATCTCCAGGGACAACGCCAAGAACACC |
| | | | CTGTATCTGCAAATGAACAGCCTGAGGGCCGAGGA |
| | | | CACGGCCCTGTATTACTGTGCGAGA |
| | HFR4 (DNA) | 112 | TGGGGCCAGGGCACCCTGGTCACTGTCTCCTCA |
| Girentuximab | HCDR1 (protein) | 113 | NYYMS |
| humanised VH5 | HCDR2 (protein) | 114 | AINSDGGITYYLDTVKG |
| Variable Heavy | HCDR3 (protein) | 115 | HRSGYFSMDY |
| chain | VH (protein) | 116 | EVQLVESGGGLVKPGGSLRLSCAASGFTFSNYYMSW |
| | | | VRQAPGKGLEWVAAINSDGGITYYLDTVKGRFTISRDN |
| | | | AKNTLYLQMNSLRAEDTALYYCARHRSGYFSMDYWG |
| | | | QGTLVTVSS |
| | HCDR1 (DNA) | 117 | AACTACTACATGAGC |
| | HCDR2 (DNA) | 118 | GCCATTAACAGTGACGGTGGCATCACCTACTACCTG |
| | | | GACACCGTGAAGGGC |
| | HCDR3 (DNA) | 119 | CACAGGAGCGGCTACTTCTCTATGGACTAC |
| | VH (DNA) | 120 | GAGGTGCAGCTGGTGGAGTCTGGGGGAGGCTTGGT |
| | | | CAAGCCTGGAGGATCCCTGAGACTCTCCTGTGCAG |
| | | | CCTCTGGATTCACCTTCAGTAACTACTACATGAGCT |
| | | | GGGTGCGCCAGGCTCCAGGGAAGGGGCTGGAGTG |
| | | | GGTTGCCGCCATTAACAGTGACGGTGGCATCACCTA |
| | | | CTACCTGGACACCGTGAAGGGCCGATTCACCATCTC |
| | | | CAGGGACAACGCCAAGAACACCCTGTATCTGCAAAT |
| | | | GAACAGCCTGAGGGCCGAGGACACGGCCCTGTATT |
| | | | ACTGTGCGAGACACAGGAGCGGCTACTTCTCTATGG |
| | | | ACTACTGGGGCCAGGGCACCCTGGTCACTGTCTCC |
| | | | TCA |
| | HFR1 (protein) | 121 | EVQLVESGGGLVKPGGSLRLSCAASGFTFS |
| | HFR2 (protein) | 122 | WVRQAPGKGLEWVA |
| | HFR3 (protein) | 123 | RFTISRDNAKNTLYLQMNSLRAEDTALYYCAR |
| | HFR4 (protein) | 124 | WGQGTLVTVSS |
| | HFR1 (DNA) | 125 | GAGGTGCAGCTGGTGGAGTCTGGGGGAGGCTTGGT |
| | | | CAAGCCTGGAGGATCCCTGAGACTCTCCTGTGCAG |
| | | | CCTCTGGATTCACCTTCAGT |
| | HFR2 (DNA) | 126 | TGGGTGCGCCAGGCTCCAGGGAAGGGGCTGGAGT |
| | | | GGGTTGCC |
| | HFR3 (DNA) | 127 | CGATTCACCATCTCCAGGGACAACGCCAAGAACACC |
| | | | CTGTATCTGCAAATGAACAGCCTGAGGGCCGAGGA |
| | | | CACGGCCCTGTATTACTGTGCGAGA |
| | HFR4 (DNA) | 128 | TGGGGCCAGGGCACCCTGGTCACTGTCTCCTCA |
| Girentuximab | LCDR1 (protein) | 129 | KASQNVVSAVA |
| Chimeric Vk0 | LCDR2 (protein) | 130 | SASNRYT |
| Variable light | LCDR3 (protein) | 131 | QQYSNYPWT |
| chain | VL (protein) | 132 | DIVMTQSQRFMSTTVGDRVSITCKASQNVVSAVAWYQ |
| | | | QKPGQSPKLLIYSASNRYTGVPDRFTGSGSGTDFTLTI |
| | | | SNMQSEDLADFFCQQYSNYPWTFGGGTKLEIK |
| | LCDR1 (DNA) | 133 | AAGGCCAGTCAGAATGTGGTTTCTGCTGTTGCC |
| | LCDR2 (DNA) | 134 | TCAGCATCCAATCGGTACACT |
| | LCDR3 (DNA) | 135 | CAACAATATAGCAACTATCCGTGGACG |
| | VL (DNA) | 136 | GACATTGTGATGACCCAGTCTCAAAGATTCATGTCC |
| | | | ACAACAGTAGGAGACAGGGTCAGCATCACCTGCAA |
| | | | GGCCAGTCAGAATGTGGTTTCTGCTGTTGCCTGGTA |
| | | | TCAACAGAAACCAGGACAATCTCCTAAACTACTGATT |
| | | | TACTCAGCATCCAATCGGTACACTGGAGTCCCTGAT |
| | | | CGCTTCACAGGCAGTGGATCTGGGACAGATTTCACT |
| | | | CTCACCATTAGCAATATGCAGTCTGAAGACCTGGCT |
| | | | GATTTTTTCTGTCAACAATATAGCAACTATCCGTGGA |
| | | | CGTTCGGTGGAGGCACCAAGCTGGAAATCAAA |
| | LFR1 | 137 | DIVMTQSQRFMSTTVGDRVSITC |
| | (protein) | | |
| | LFR2 (protein) | 138 | WYQQKPGQSPKLLIY |
| | LFR3 (protein) | 139 | GVPDRFTGSGSGTDFTLTISNMQSEDLADFFC |
| | LFR4 | 140 | FGGGTKLEIK |
| | (protein) | | |
| | LFR1 | 141 | GACATTGTGATGACCCAGTCTCAAAGATTCATGTCC |
| | (DNA) | | ACAACAGTAGGAGACAGGGTCAGCATCACCTGC |
| | LFR2 (DNA) | 142 | TGGTATCAACAGAAACCAGGACAATCTCCTAAACTA |
| | | | CTGATTTAC |
| | LFR3 (DNA) | 143 | GGAGTCCCTGATCGCTTCACAGGCAGTGGATCTGG |
| | | | GACAGATTTCACTCTCACCATTAGCAATATGCAGTCT |
| | | | GAAGACCTGGCTGATTTTTTCTGT |
| | LFR4 | 144 | TTCGGTGGAGGCACCAAGCTGGAAATCAAA |
| | (DNA) | | |
| Girentuximab | LCDR1 (protein) | 145 | KASQNVVSAVA |
| Humanised Vk1 | LCDR2 | 146 | SASNRYT |
| Variable light | (protein) | | |
| chain | LCDR3 (protein) | 147 | QQYSNYPWT |
| | VL (protein) | 148 | DIVMTQSPSFLSASVGDRVTITCKASQNVVSAVAWYQ |
| | | | QKPGQAPKLLIYSASNRYTGVPDRFTGSGSGTDFTLTI |
| | | | SSLQSEDLADYFCQQYSNYPWTFGGGTKVEIK |
| | LCDR1 (DNA) | 149 | AAGGCAAGTCAGAACGTGGTGAGTGCTGTGGCC |
| | LCDR2 (DNA) | 150 | AGCGCCTCCAACAGGTACACC |
| | LCDR3 (DNA) | 151 | CAACAGTACAGCAATTACCCTTGGACG |
| | VL (DNA) | 152 | GACATCGTGATGACCCAGTCTCCATCCTTCCTGTCT |
| | | | GCATCTGTAGGAGACAGAGTCACCATCACTTGCAAG |
| | | | GCAAGTCAGAACGTGGTGAGTGCTGTGGCCTGGTA |
| | | | TCAGCAGAAACCAGGGCAGGCTCCTAAGCTCCTGAT |
| | | | CTATAGCGCCTCCAACAGGTACACCGGGGTCCCAG |
| | | | ACAGGTTCACCGGCAGTGGATCTGGGACAGATTTCA |
| | | | CTCTCACCATCAGCAGCCTGCAGAGCGAAGATCTG |
| | | | GCAGACTATTTCTGTCAACAGTACAGCAATTACCCTT |
| | | | GGACGTTCGGCGGCGGGACCAAGGTGGAAATCAAA |
| | LFR1 | 153 | DIVMTQSPSFLSASVGDRVTITC |
| | (protein) | | |
| | LFR2 (protein) | 154 | WYQQKPGQAPKLLIY |
| | LFR3 (protein) | 155 | GVPDRFTGSGSGTDFTLTISSLQSEDLADYFC |
| | LFR4 | 156 | FGGGTKVEIK |
| | (protein) | | |
| | LFR1 | 157 | GACATCGTGATGACCCAGTCTCCATCCTTCCTGTCT |
| | (DNA) | | GCATCTGTAGGAGACAGAGTCACCATCACTTGC |
| | LFR2 (DNA) | 158 | TGGTATCAGCAGAAACCAGGGCAGGCTCCTAAGCT |
| | | | CCTGATCTAT |
| | LFR3 (DNA) | 159 | GGGGTCCCAGACAGGTTCACCGGCAGTGGATCTGG |
| | | | GACAGATTTCACTCTCACCATCAGCAGCCTGCAGAG |
| | | | CGAAGATCTGGCAGACTATTTCTGT |
| | LFR4 | 160 | TTCGGCGGCGGGACCAAGGTGGAAATCAAA |
| | (DNA) | | |
| Girentuximab | LCDR1 (protein) | 161 | KASQNVVSAVA |
| Humanised Vk2 | LCDR2 | 162 | SASNRYT |
| Variable light | (protein) | | |
| chain | LCDR3 (protein) | 163 | QQYSNYPWT |
| | VL (protein) | 164 | DIVMTQSPSSLSASVGDRVTITCKASQNVVSAVAWYQ |
| | | | QKPGQAPRLLIYSASNRYTGVPDRFTGSGSGTDFTLTI |
| | | | SSLQAEDLADYFCQQYSNYPWTFGGGTKVEIK |
| | LCDR1 (DNA) | 165 | AAGGCAAGTCAGAACGTGGTGAGTGCTGTGGCC |
| | LCDR2 (DNA) | 166 | AGCGCCTCCAACAGGTACACC |
| | LCDR3 (DNA) | 167 | CAACAGTACAGCAATTACCCTTGGACG |
| | VL (DNA) | 168 | GACATCGTGATGACCCAGTCTCCATCCAGCCTGTCT |
| | | | GCATCTGTAGGAGACAGAGTCACCATCACTTGCAAG |
| | | | GCAAGTCAGAACGTGGTGAGTGCTGTGGCCTGGTA |
| | | | TCAGCAGAAACCAGGGCAGGCTCCTAGGCTCCTGA |
| | | | TCTATAGCGCCTCCAACAGGTACACCGGGGTCCCA |
| | | | GACAGGTTCACCGGCAGTGGATCTGGGACAGATTT |
| | | | CACTCTCACCATCAGCAGCCTGCAGGCCGAAGATCT |
| | | | GGCAGACTATTTCTGTCAACAGTACAGCAATTACCC |
| | | | TTGGACGTTCGGCGGCGGGACCAAGGTGGAAATCA |
| | | | AA |
| | LFR1 | 169 | DIVMTQSPSSLSASVGDRVTITC |
| | (protein) | | |
| | LFR2 (protein) | 170 | WYQQKPGQAPRLLIY |
| | LFR3 (protein) | 171 | GVPDRFTGSGSGTDFTLTISSLQAEDLADYFC |
| | LFR4 | 172 | FGGGTKVEIK |
| | (protein) | | |
| | LFR1 | 173 | GACATCGTGATGACCCAGTCTCCATCCAGCCTGTCT |
| | (DNA) | | GCATCTGTAGGAGACAGAGTCACCATCACTTGC |
| | LFR2 (DNA) | 174 | TGGTATCAGCAGAAACCAGGGCAGGCTCCTAGGCT |
| | | | CCTGATCTAT |
| | LFR3 (DNA) | 175 | GGGGTCCCAGACAGGTTCACCGGCAGTGGATCTGG |
| | | | GACAGATTTCACTCTCACCATCAGCAGCCTGCAGGC |
| | | | CGAAGATCTGGCAGACTATTTCTGT |
| | LFR4 | 176 | TTCGGCGGCGGGACCAAGGTGGAAATCAAA |
| | (DNA) | | |
| Girentuximab | LCDR1 (protein) | 177 | KASQNVVSAVA |
| Humanised Vk3 | LCDR2 | 178 | SASNRYT |
| Variable light | (protein) | | |
| chain | LCDR3 (protein) | 179 | QQYSNYPWT |
| | VL (protein) | 180 | DIQMTQSPSSLSASVGDRVTITCKASQNVVSAVAWYQ |
| | | | QKPGQAPRLLIYSASNRYTGVPDRFSGSGSGTDFTLTI |
| | | | SSLQAEDLADYFCQQYSNYPWTFGGGTKVEIK |
| | LCDR1 (DNA) | 181 | AAGGCAAGTCAGAACGTGGTGAGTGCTGTGGCC |
| | LCDR2 (DNA) | 182 | AGCGCCTCCAACAGGTACACC |
| | LCDR3 (DNA) | 183 | CAACAGTACAGCAATTACCCTTGGACG |
| | VL (DNA) | 184 | GACATCCAGATGACCCAGTCTCCATCCAGCCTGTCT |
| | | | GCATCTGTAGGAGACAGAGTCACCATCACTTGCAAG |
| | | | GCAAGTCAGAACGTGGTGAGTGCTGTGGCCTGGTA |
| | | | TCAGCAGAAACCAGGGCAGGCTCCTAGGCTCCTGA |
| | | | TCTATAGCGCCTCCAACAGGTACACCGGGGTCCCA |
| | | | GACAGGTTCAGCGGCAGTGGATCTGGGACAGATTT |
| | | | CACTCTCACCATCAGCAGCCTGCAGGCCGAAGATCT |
| | | | GGCAGACTATTTCTGTCAACAGTACAGCAATTACCC |
| | | | TTGGACGTTCGGCGGCGGGACCAAGGTGGAAATCA |
| | | | AA |
| | LFR1 | 185 | DIQMTQSPSSLSASVGDRVTITC |
| | (protein) | | |
| | LFR2 (protein) | 186 | WYQQKPGQAPRLLIY |
| | LFR3 (protein) | 187 | GVPDRFSGSGSGTDFTLTISSLQAEDLADYFC |
| | LFR4 | 188 | FGGGTKVEIK |
| | (protein) | | |
| | LFR1 | 189 | GACATCCAGATGACCCAGTCTCCATCCAGCCTGTCT |
| | (DNA) | | GCATCTGTAGGAGACAGAGTCACCATCACTTGC |
| | LFR2 (DNA) | 190 | TGGTATCAGCAGAAACCAGGGCAGGCTCCTAGGCT |
| | | | CCTGATCTAT |
| | LFR3 (DNA) | 191 | GGGGTCCCAGACAGGTTCAGCGGCAGTGGATCTGG |
| | | | GACAGATTTCACTCTCACCATCAGCAGCCTGCAGGC |
| | | | CGAAGATCTGGCAGACTATTTCTGT |
| | LFR4 | 192 | TTCGGCGGCGGGACCAAGGTGGAAATCAAA |
| | (DNA) | | |
| Girentuximab | LCDR1 (protein) | 193 | KASQNVVSAVA |
| Humanised Vk4 | LCDR2 | 194 | SASNRYT |
| Variable light | (protein) | | |
| chain | LCDR3 (protein) | 195 | QQYSNYPWT |
| | VL (protein) | 196 | DIQMTQSPSSLSASVGDRVTITCKASQNVVSAVAWYQ |
| | | | QKPGQAPRLLIYSASNRYTGVPDRFSGSGSGTDFTLTI |
| | | | SSLQAEDLADYYCQQYSNYPWTFGGGTKVEIK |
| | LCDR1 (DNA) | 197 | AAGGCAAGTCAGAACGTGGTGAGTGCTGTGGCC |
| | LCDR2 (DNA) | 198 | AGCGCCTCCAACAGGTACACC |
| | LCDR3 (DNA) | 199 | CAACAGTACAGCAATTACCCTTGGACG |
| | VL (DNA) | 200 | GACATCCAGATGACCCAGTCTCCATCCAGCCTGTCT |
| | | | GCATCTGTAGGAGACAGAGTCACCATCACTTGCAAG |
| | | | GCAAGTCAGAACGTGGTGAGTGCTGTGGCCTGGTA |
| | | | TCAGCAGAAACCAGGGCAGGCTCCTAGGCTCCTGA |
| | | | TCTATAGCGCCTCCAACAGGTACACCGGGGTCCCA |
| | | | GACAGGTTCAGCGGCAGTGGATCTGGGACAGATTT |
| | | | CACTCTCACCATCAGCAGCCTGCAGGCCGAAGATCT |
| | | | GGCAGACTATTACTGTCAACAGTACAGCAATTACCC |
| | | | TTGGACGTTCGGCGGCGGGACCAAGGTGGAAATCA |
| | | | AA |
| | LFR1 | 201 | DIQMTQSPSSLSASVGDRVTITC |
| | (protein) | | |
| | LFR2 (protein) | 202 | WYQQKPGQAPRLLIY |
| | LFR3 (protein) | 203 | GVPDRFSGSGSGTDFTLTISSLQAEDLADYYC |
| | LFR4 | 204 | FGGGTKVEIK |
| | (protein) | | |
| | LFR1 | 205 | GACATCCAGATGACCCAGTCTCCATCCAGCCTGTCT |
| | (DNA) | | GCATCTGTAGGAGACAGAGTCACCATCACTTGC |
| | LFR2 (DNA) | 206 | TGGTATCAGCAGAAACCAGGGCAGGCTCCTAGGCT |
| | | | CCTGATCTAT |
| | LFR3 (DNA) | 207 | GGGGTCCCAGACAGGTTCAGCGGCAGTGGATCTGG |
| | | | GACAGATTTCACTCTCACCATCAGCAGCCTGCAGGC |
| | | | CGAAGATCTGGCAGACTATTACTGT |
| | LFR4 | 208 | TTCGGCGGCGGGACCAAGGTGGAAATCAAA |
| | (DNA) | | |
| Girentuximab | LCDR1 (protein) | 209 | KASQNVVSAVA |
| Humanised Vk5 | LCDR2 | 210 | SASNRYT |
| Variable light | (protein) | | |
| chain | LCDR3 (protein) | 211 | QQYSNYPWT |
| | VL (protein) | 212 | DIQMTQSPSSLSASVGDRVTITCKASQNVVSAVAWYQ |
| | | | QKPGQAPRRLIYSASNRYTGVPDRFSGSGSGTDFTLTI |
| | | | SSLQAEDLADYYCQQYSNYPWTFGGGTKVEIK |
| | LCDR1 (DNA) | 213 | AAGGCAAGTCAGAACGTGGTGAGTGCTGTGGCC |
| | LCDR2 (DNA) | 214 | AGCGCCTCCAACAGGTACACC |
| | LCDR3 (DNA) | 215 | CAACAGTACAGCAATTACCCTTGGACG |
| | VL (DNA) | 216 | GACATCCAGATGACCCAGTCTCCATCCAGCCTGTCT |
| | | | GCATCTGTAGGAGACAGAGTCACCATCACTTGCAAG |
| | | | GCAAGTCAGAACGTGGTGAGTGCTGTGGCCTGGTA |
| | | | TCAGCAGAAACCAGGGCAGGCTCCTAGGAGGCTGA |
| | | | TCTATAGCGCCTCCAACAGGTACACCGGGGTCCCA |
| | | | GACAGGTTCAGCGGCAGTGGATCTGGGACAGATTT |
| | | | CACTCTCACCATCAGCAGCCTGCAGGCCGAAGATCT |
| | | | GGCAGACTATTACTGTCAACAGTACAGCAATTACCC |
| | | | TTGGACGTTCGGCGGCGGGACCAAGGTGGAAATCA |
| | | | AA |
| | LFR1 | 217 | DIQMTQSPSSLSASVGDRVTITC |
| | (protein) | | |
| | LFR2 (protein) | 218 | WYQQKPGQAPRRLIY |
| | LFR3 (protein) | 219 | GVPDRFSGSGSGTDFTLTISSLQAEDLADYYC |
| | LFR4 | 220 | FGGGTKVEIK |
| | (protein) | | |
| | LFR1 | 221 | GACATCCAGATGACCCAGTCTCCATCCAGCCTGTCT |
| | (DNA) | | GCATCTGTAGGAGACAGAGTCACCATCACTTGC |
| | LFR2 (DNA) | 222 | TGGTATCAGCAGAAACCAGGGCAGGCTCCTAGGAG |
| | | | GCTGATCTAT |
| | LFR3 (DNA) | 223 | GGGGTCCCAGACAGGTTCAGCGGCAGTGGATCTGG |
| | | | GACAGATTTCACTCTCACCATCAGCAGCCTGCAGGC |
| | | | CGAAGATCTGGCAGACTATTACTGT |
| | LFR4 | 224 | TTCGGCGGCGGGACCAAGGTGGAAATCAAA |
| | (DNA) | | |
| Girentuximab | IgG1 HC | 225 | ASTKGPSVFPLAPSSKSTSGGTAALGCLVKDYFPEPVT |
| unmodified | (protein) | | VSWNSGALTSGVHTFPAVLQSSGLYSLSSVVTVPSSS |
| human IgG1 | | | LGTQTYICNVNHKPSNTKVDKKVEPKSCDKTHTCPPC |
| constant chain | | | PAPELLGGPSVFLFPPKPKDTLMISRTPEVTCVVVDVS |
| region | | | HEDPEVKFNWYVDGVEVHNAKTKPREEQYNSTYRVV |
| | | | SVLTVLHQDWLNGKEYKCKVSNKALPAPIEKTISKAKG |
| | | | QPREPQVYTLPPSRDELTKNQVSLTCLVKGFYPSDIAV |
| | | | EWESNGQPENNYKTTPPVLDSDGSFFLYSKLTVDKSR |
| | | | WQQGNVFSCSVMHEALHNHYTQKSLSLSPGK |
| Girentuximab | IgG1 H310A | 226 | ASTKGPSVFPLAPSSKSTSGGTAALGCLVKDYFPEPVT |
| modified human | H435Q HC | | VSWNSGALTSGVHTFPAVLQSSGLYSLSSVVTVPSSS |
| IgG1 constant | (protein) | | LGTQTYICNVNHKPSNTKVDKKVEPKSCDKTHTCPPC |
| chain region | | | PAPELLGGPSVFLFPPKPKDTLMISRTPEVTCVVVDVS |
| | | | HEDPEVKFNWYVDGVEVHNAKTKPREEQYNSTYRVV |
| | | | SVLTVLAQDWLNGKEYKCKVSNKALPAPIEKTISKAKG |
| | | | QPREPQVYTLPPSRDELTKNQVSLTCLVKGFYPSDIAV |
| | | | EWESNGQPENNYKTTPPVLDSDGSFFLYSKLTVDKSR |
| | | | WQQGNVFSCSVMHEALHNQYTQKSLSLSPGK |
| Girentuximab | IgG4 S228P | 227 | ASTKGPSVFPLAPCSRSTSESTAALGCLVKDYFPEPVT |
| modified human | L235E HC | | VSWNSGALTSGVHTFPAVLQSSGLYSLSSVVTVPSSS |
| IgG4 constant | (protein) | | LGTKTYTCNVDHKPSNTKVDKRVESKYGPPCPPCPAP |
| chain region | | | EFEGGPSVFLFPPKPKDTLMISRTPEVTCVVVDVSQED |
| | | | PEVQFNWYVDGVEVHNAKTKPREEQFNSTYRVVSVLT |
| | | | VLHQDWLNGKEYKCKVSNKGLPSSIEKTISKAKGQPR |
| | | | EPQVYTLPPSQEEMTKNQVSLTCLVKGFYPSDIAVEW |
| | | | ESNGQPENNYKTTPPVLDSDGSFFLYSRLTVDKSRWQ |
| | | | EGNVFSCSVMHEALHNHYTQKSLSLSLGK |
| Girentuximab | IgG4 S228P | 228 | ASTKGPSVFPLAPCSRSTSESTAALGCLVKDYFPEPVT |
| modified human | L235E H310A | | VSWNSGALTSGVHTFPAVLQSSGLYSLSSVVTVPSSS |
| IgG4 constant | H435Q HC | | LGTKTYTCNVDHKPSNTKVDKRVESKYGPPCPPCPAP |
| chain region | (protein) | | EFEGGPSVFLFPPKPKDTLMISRTPEVTCVVVDVSQED |
| | | | PEVQFNWYVDGVEVHNAKTKPREEQFNSTYRVVSVLT |
| | | | VLAQDWLNGKEYKCKVSNKGLPSSIEKTISKAKGQPRE |
| | | | PQVYTLPPSQEEMTKNQVSLTCLVKGFYPSDIAVEWE |
| | | | SNGQPENNYKTTPPVLDSDGSFFLYSRLTVDKSRWQE |
| | | | GNVFSCSVMHEALHNQYTQKSLSLSLGK |
| Girentuximab | Human κ LC | 229 | RTVAAPSVFIFPPSDEQLKSGTASVVCLLNNFYPREAK |
| kappa light chain | constant region | | VQWKVDNALQSGNSQESVTEQDSKDSTYSLSSTLTLS |
| constant region | | | KADYEKHKVYACEVTHQGLSSPVTKSFNRGEC |
| Girentuximab | IgG1 VH4 heavy | 230 | EVQLVESGGGLVKPGGSLRLSCAASGFTFSNYYMSW |
| native IgG1 | chain | | VRQAPGKGLELVAAINSDGGITYYLDTVKGRFTISRDN |
| allotype Gm | | | AKNTLYLQMNSLRAEDTALYYCARHRSGYFSMDYWG |
| (1,17) VH4 | | | QGTLVTVSSASTKGPSVFPLAPSSKSTSGGTAALGCLV |
| heavy chain | | | KDYFPEPVTVSWNSGALTSGVHTFPAVLQSSGLYSLS |
| | | | SVVTVPSSSLGTQTYICNVNHKPSNTKVDKKVEPKSCD |
| | | | KTHTCPPCPAPELLGGPSVFLFPPKPKDTLMISRTPEV |
| | | | TCVVVDVSHEDPEVKFNWYVDGVEVHNAKTKPREEQ |
| | | | YNSTYRVVSVLTVLHQDWLNGKEYKCKVSNKALPAPIE |
| | | | KTISKAKGQPREPQVYTLPPSRDELTKNQVSLTCLVKG |
| | | | FYPSDIAVEWESNGQPENNYKTTPPVLDSDGSFFLYS |
| | | | KLTVDKSRWQQGNVFSCSVMHEALHNHYTQKSLSLS |
| | | | PGK |
| hGirentuximab | FcRn-null IgG1 | 231 | EVQLVESGGGLVKPGGSLRLSCAASGFTFSNYYMSW |
| IgG1 allotype | allotype H130A | | VRQAPGKGLELVAAINSDGGITYYLDTVKGRFTISRDN |
| Gm (1,17) | H435Q | | AKNTLYLQMNSLRAEDTALYYCARHRSGYFSMDYWG |
| RADmAb VH4 | | | QGTLVTVSSASTKGPSVFPLAPSSKSTSGGTAALGCLV |
| heavy chain | | | KDYFPEPVTVSWNSGALTSGVHTFPAVLQSSGLYSLS |
| | | | SVVTVPSSSLGTQTYICNVNHKPSNTKVDKKVEPKSCD |
| | | | KTHTCPPCPAPELLGGPSVFLFPPKPKDTLMISRTPEV |
| | | | TCVVVDVSHEDPEVKFNWYVDGVEVHNAKTKPREEQ |
| | | | YNSTYRVVSVLTVLAQDWLNGKEYKCKVSNKALPAPIE |
| | | | KTISKAKGQPREPQVYTLPPSRDELTKNQVSLTCLVKG |
| | | | FYPSDIAVEWESNGQPENNYKTTPPVLDSDGSFFLYS |
| | | | KLTVDKSRWQQGNVFSCSVMHEALHNQYTQKSLSLS |
| | | | PGK |
| hGirentuximab | FcRγ-null IgG4 | 232 | EVQLVESGGGLVKPGGSLRLSCAASGFTFSNYYMSW |
| IgG4 RADmAb | S228P L235E | | VRQAPGKGLELVAAINSDGGITYYLDTVKGRFTISRDN |
| VH4 heavy | | | AKNTLYLQMNSLRAEDTALYYCARHRSGYFSMDYWG |
| chain | | | QGTLVTVSSASTKGPSVFPLAPCSRSTSESTAALGCLV |
| | | | KDYFPEPVTVSWNSGALTSGVHTFPAVLQSSGLYSLS |
| | | | SVVTVPSSSLGTKTYTCNVDHKPSNTKVDKRVESKYG |
| | | | PPCPPCPAPEFEGGPSVFLFPPKPKDTLMISRTPEVTC |
| | | | VVVDVSQEDPEVQFNWYVDGVEVHNAKTKPREEQFN |
| | | | STYRVVSVLTVLHQDWLNGKEYKCKVSNKGLPSSIEKT |
| | | | ISKAKGQPREPQVYTLPPSQEEMTKNQVSLTCLVKGF |
| | | | YPSDIAVEWESNGQPENNYKTTPPVLDSDGSFFLYSR |
| | | | LTVDKSRWQEGNVFSCSVMHEALHNHYTQKSLSLSLG |
| | | | K |
| hGirentuximab | FcRn+FcRγ-null | 233 | EVQLVESGGGLVKPGGSLRLSCAASGFTFSNYYMSW |
| IgG4 RADmAb | IgG4 S228P | | VRQAPGKGLELVAAINSDGGITYYLDTVKGRFTISRDN |
| VH4 heavy | L235E H310A | | AKNTLYLQMNSLRAEDTALYYCARHRSGYFSMDYWG |
| chain | H435Q | | QGTLVTVSSASTKGPSVFPLAPCSRSTSESTAALGCLV |
| | | | KDYFPEPVTVSWNSGALTSGVHTFPAVLQSSGLYSLS |
| | | | SVVTVPSSSLGTKTYTCNVDHKPSNTKVDKRVESKYG |
| | | | PPCPPCPAPEFEGGPSVFLFPPKPKDTLMISRTPEVTC |
| | | | VVVDVSQEDPEVQFNWYVDGVEVHNAKTKPREEQFN |
| | | | STYRVVSVLTVLAQDWLNGKEYKCKVSNKGLPSSIEKT |
| | | | ISKAKGQPREPQVYTLPPSQEEMTKNQVSLTCLVKGF |
| | | | YPSDIAVEWESNGQPENNYKTTPPVLDSDGSFFLYSR |
| | | | LTVDKSRWQEGNVFSCSVMHEALHNQYTQKSLSLSL |
| | | | GK |
| hGirentuximab | Vκ Light Chain | 234 | DIQMTQSPSSLSASVGDRVTITCKASQNVVSAVAWYQ |
| Vκ Light Chain | | | QKPGQAPRLLIYSASNRYTGVPDRFSGSGSGTDFTLTI |
| | | | SSLQAEDLADYYCQQYSNYPWTFGGGTKVEIKRTVAA |
| | | | PSVFIFPPSDEQLKSGTASVVCLLNNFYPREAKVQWKV |
| | | | DNALQSGNSQESVTEQDSKDSTYSLSSTLTLSKADYE |
| | | | KHKVYACEVTHQGLSSPVTKSFNRGEC |

### EXAMPLES

### Example 1: Antibodies for Binding to CAIX

### Summary

Humanised antibody variable region genes were cloned into vectors encoding an unmodified human IgG1 heavy chain constant domain and human kappa light chain constant domain. The chimeric antibody was additionally cloned into vectors encoding either a modified human IgG1 heavy chain constant domain containing the mutations H310A and H435Q that abolish FcRn and Protein A binding (Andersen et al, 2012) or a modified human IgG4 heavy chain construct containing the mutations S228P (to stabilise the hinge (Angal et al, 1993)) and L235E (to remove effector function (Reddy et al, 2000)) as well as the FcRn abolishing mutations described above. Chimeric and humanised antibodies were transiently expressed in HEK EBNA cells and tested for binding to human carbonic anhydrase IX (CAIX) using Biacore single cycle kinetic analysis. Selected lead antibodies were purified by Protein A or Protein G and analysed by analytical SEC, competition ELISA and Biacore multicycle kinetic analysis.

Three lead candidate humanised antibodies with similar binding to the chimeric antibody were identified. These were subsequently cloned into vectors encoding the human IgG1 heavy chain constant domain (H310A, H435Q) and IgG4 heavy chain constant domain (S241P, L235E, H310A, H435Q) as described above. Chimeric and lead humanised antibodies were transiently expressed in CHO cells and tested for binding to human carbonic anhydrase IX (CAIX) using Biacore single cycle kinetic analysis. Antibodies were purified by either Protein A or Protein G and analysed by analytical SEC and Biacore multicycle kinetic analysis.

### Methods

### Design of Composite Human Antibody™ Variable Region Sequences

Structural models of the Girentuximab antibody V regions were produced using Swiss PDB and analysed in order to identify important “constraining” amino acids that were likely to be essential for the binding properties of the antibody. Most residues contained within the CDRs (using both Kabat and Chothia definitions) together with a number of framework residues were considered to be important. The VH and Vκ sequences of Girentuximab contain typical framework residues and CDR 1, 2 and 3 motifs that are comparable to many murine antibodies.

From the above analysis, it was considered that Composite Human sequences of Girentuximab could be created with a wide latitude for alternative residues outside of the CDRs but with only a narrow menu of possible residues within the CDR sequences.

Preliminary analysis indicated that corresponding sequence segments from several human antibodies could be combined to create CDRs similar or identical to those in the murine sequences. For regions outside of, and flanking the CDRs, a wide selection of human sequence segments were identified as possible components of the novel humanised V regions.

### CD4+ T Cell Epitope Avoidance

Based upon the structural analysis, a large preliminary set of sequence segments were identified that could be used to create Girentuximab humanised variants. These segments were selected and analysed using iTope™ technology for in silico analysis of peptide binding to human MHC class II alleles (Perry et al, 2008), and using the TCED™ of known antibody sequence-related T cell epitopes (Bryson et al, 2010). Sequence segments that were identified as significant non-human germline binders to human MHC class II or that scored significant hits against the TCED™ were discarded. This resulted in a reduced set of segments, and combinations of these were again analysed, as above, to ensure that the junctions between segments did not contain potential T cell epitopes. Selected sequence segments were assembled into complete V region sequences that were devoid of significant T cell epitopes. Five heavy chain (VH0 (Chimeric), VH1, VH3, VH4 and VH5) and six light chain (Vκ0 (Chimeric), Vκ1 to Vκ5) sequences were then chosen for gene synthesis and expression in mammalian cells (see Table 2).

### Construction of Chimeric Antibodies and Humanised Variants

The chimeric VH0 and Vκ0 Girentuximab sequences and its humanised variants were synthesised with flanking restriction enzyme sites for cloning into Abzena's pANT expression vector system for unmodified human IgG1 heavy chain (allotype G1m 1,17; equivalent to the Girentuximab allotype) (pANT68) and kappa light chain (pANT13.2). The VH regions were cloned between the Mlu I and Hind III restriction sites, and the Vκ regions were cloned between the BssH II and BamH I restriction sites. All constructs were confirmed by sequencing.

The FcRn abolishing mutations, H310A and H435Q, were incorporated by site directed mutagenesis into expression vectors for either human IgG1 heavy chain (equivalent allotype to Girentuximab) (pANT69) or for human IgG4 (S228P, L235E) heavy chain (pANT70). The VH0 was cloned into these two vectors in addition to pANT36.2 which encodes IgG4 (S228P, L235E).

### Expression of Antibodies in HEK293 EBNA Cells

Chimeric Girentuximab (VH0/VNκ0), two control antibodies (VH0/Vκ1, VH1/Vκ0) and combinations of VH and Vκ chains (a total of 20 pairings) were transiently expressed as IgG1 in HEK EBNA cells (LGC Standards, Teddington, UK) using a PEI transfection method. Cells were incubated for seven days post-transfection. Additionally, HEK EBNA cells were similarly transfected with chimeric Girentuximab VH0 and Vκ0 in an IgG1 (H310A, H435Q) heavy chain vector, an IgG4 (S228P, L235E) heavy chain vector and an IgG4 (S228P, L235E H310A, H435Q) heavy chain vector. Supernatant antibody titres were determined by ELISA.

### Kinetic Analysis of Chimeric and Humanised Variant Binding to Carbonic Anhydrase IX

In order to assess the binding of all Girentuximab Composite Human Antibody™ variants, single cycle kinetic analysis was performed on supernatants from transiently transfected HEK EBNA cell cultures. Kinetic experiments were performed on a Biacore T200 (serial no. 1909913) running Biacore T200 Control software V2.0.1 and Evaluation software V3.0 (GE Healthcare, Uppsala, Sweden). All single cycle kinetic experiments were run at 25° C. with HBS-P+ running buffer (pH 7.4) (GE Healthcare, Little Chalfont, UK). For direct comparison, all antibodies were captured on a Protein G chip (GE Healthcare, Uppsala, Sweden).

Antibodies were diluted in running buffer to a final concentration of 0.5 μg/ml, based on concentrations assessed by ELISA titre. At the start of each cycle, antibodies were loaded onto Fc2, Fc3 and Fc4 of the Protein G chip (GE Healthcare, Little Chalfont, UK). IgGs were captured at a flow rate of 10 μl/min to give an immobilisation level (RL) of ˜79 RU, the theoretical value to obtain an Rmax of ˜50 RU. The surface was then allowed to stabilise. Single cycle kinetic data was obtained with Carbonic Anhydrase IX (CAIX, Stratech, Newmarket, UK) as the analyte at a flow rate of 30 μl/min to minimise any potential mass transport limitations. Multiple repeats with the reference chimeric antibody (VH0/Vκ0 IgG1) were performed to check the stability of the surface and analyte over the kinetic cycles. The signal from the reference channel Fc1 (no antibody) was subtracted from that of Fc2, Fc3 and Fc4 to correct for differences in non-specific binding to a reference surface. A four point, two-fold dilution range from 3.125 nM to 25 nM CAIX without regeneration between each concentration was used. The association phase for the four injections of increasing concentrations of CAIX was monitored for 200 seconds each time and a single dissociation phase was measured for 300 seconds following the last injection of CAIX. Regeneration of the Protein G surface was conducted using an injection of 10 mM glycine-HCL pH 1.5 followed by an injection of 10 mM glycine-HCL pH 1.5 containing 0.5% P20.

Variants were analysed with the reference chimeric (VH0/Vκ0 IgG1) used for the calculation of the relative KD. The signal from each antibody blank run was subtracted to correct for differences in surface stability. Single cycle kinetics data demonstrated that all humanised variants bound to CAIX within two-fold of the reference chimeric antibody, except those containing a VH5 and/or a Vκ5, as these variants displayed lower affinities to CAIX.

Purification of chimeric and lead humanised antibodies Six IgG1 lead antibodies, VH3/Vκ2, VH3/Vκ3, VH3/Vκ4, VH4/Vκ2, VH4/Vκ3 and VH4/Vκ4 were purified based on humanness and single cycle kinetics data. The chimeric IgG1 antibody, lead humanised IgG1 antibodies and chimeric IgG4 (S228P, L235E) antibody were purified from cell culture supernatants using Protein A sepharose columns (GE Healthcare, Little Chalfont, UK). The chimeric IgG1 (H310A, H435Q) and IgG4 (S228P, L325E, H310A, H435Q) antibodies were purified from cell culture supernatant using HiTrap™ Protein G HP columns (GE Healthcare, Little Chalfont, UK) as the H310A H435Q double mutation has been shown to adversely affect binding to some Protein A resins. All antibodies were buffer exchanged into 1×PBS pH 7.2 and quantified by OD280 nm using an extinction coefficient (Ec(0.1%)) based on the predicted amino acid sequence. Antibodies were analysed by reducing SDS-PAGE of 2 μg loaded antibody and bands corresponding to the profile of a typical antibody were observed.

Multicycle kinetics analysis of chimeric and lead antibodies Multicycle kinetics analysis was performed on the purified chimeric antibodies and six lead IgG1 antibodies using a Biacore T200 (serial no. 1909913) instrument running Biacore T200 Evaluation Software V3.0.1 (Uppsala, Sweden) to establish an accurate affinity for carbonic anhydrase IX. The purified antibodies were diluted to a concentration of 1 μg/ml in HBS-P+. At the start of each cycle, each antibody was captured on the Protein G surface to give an RL of ˜79 RU. Following capture, the surface was allowed to stabilise. Kinetic data was obtained using a flow rate of 30 μl/min to minimise any potential mass transfer effects. For the kinetics analysis, carbonic anhydrase IX (CAIX) was used. Multiple repeats of the blank (CAIX) and a repeat of a single concentration of the analyte were programmed into the kinetic run in order to check the stability of both the surface and analyte over the kinetic cycles. For kinetic analysis, a two-fold dilution range was selected from 50 nM to 0.078 nM CAIX. The association phase of CAIX was monitored for 280 seconds and the dissociation phase was monitored for 300 seconds. Regeneration of the Protein G surface was conducted using an injection of 10 mM glycine-HCL pH 1.5 followed by an injection of 10 mM glycine-HCL pH 1.5 containing 0.5% P20. The signal from the reference channel Fc1 was subtracted from that of Fc2, Fc3 and Fc4 to correct for differences in non-specific binding to a reference surface, and a global Rmax parameter was used in the 1-to-1 binding model. The relative KD was calculated by dividing the KD of the humanised variants by that of the chimeric antibody on the same chip. All the lead humanised IgG1 variants and the chimeric antibodies on different IgG backbones showed relative KDs within 2-fold of the chimeric IgG1 antibody.

### Carbonic Anhydrase IX Competition ELISA

Lead purified IgG1 variants and chimeric antibodies expressed with different IgG constant domains (IgG1, IgG1 (H310A, H435Q), IgG4 (S228P, L235E) and IgG4 (S228P, L235E, H310A, H435Q) were tested for their binding to CAIX using competition against biotinylated (Biotinylation kit from Innova Biosciences, Cambridge, UK) chimeric Girentuximab IgG1. An irrelevant human IgG1 antibody was tested as a negative control for binding to CAIX.

CAIX was diluted in 1×PBS to 0.5 μg/ml and 100 μl/well was coated overnight at 4° C. on a 96-well ELISA plate. The following day, the plate was washed 3× with 1×PBS/0.05% Tween (PBST) and blocked with 200 μl of 2% BSA/PBS for one hour at room temperature. In a 96-well dilution plate a fixed concentration of biotinylated Girentuximab chimeric IgG1 antibody (0.3 μg/ml final concentration) was added in equal volume to a three-fold titration series of test antibody (starting from 20 μg/ml (10 μg/ml final concentration) diluted in blocking buffer).

After washing the plate 3× with PBS-T, 100 μl of chimeric/test antibody mix was added to the ELISA plate.

After one hour incubation at room temperature, the plate was washed 3× with PBS-T and 100 μl of streptavidin-peroxidase conjugated secondary antibody (Sigma, Dorset, UK) diluted 1:1000 in PBS-T was applied for one hour at room temperature to detect bound biotinylated Girentuximab chimeric IgG1 antibody. For colour development, the plate was washed 3× with PBS-T following which 100 μl of TMB substrate was added and incubated for approximately five minutes at room temperature. The reaction was stopped with 50 μl of 3.0 M hydrochloric acid and absorbance was read immediately using a Dynex plate reader at 450 nm.

IC₅₀ values were calculated for each variant and relative IC50 values were calculated by dividing the IC50 of the humanised variant by that of the Girentuximab chimeric IgG1 antibody assayed on the same plate. All lead humanised variants and chimeric antibodies on different backbones demonstrated IC50 values within two-fold of the Girentuximab chimeric IgG1 antibody.

From analysis of all the data generated (including iTope™ analysis, percentage humanness, multicycle kinetics data and competition ELISA data), the three antibodies, VH3/Vκ4, VH4/Vκ3 and VH4/Vκ4, were chosen as leads for further expression and analysis as IgG1, IgG1 (H310A, H435Q) and IgG4 (S228P, L235E, H310A, H435Q).

### Expression of Lead Humanised Girentuximab Antibodies in CHO Cells

Heavy chain variable domains were cloned into the Fc null vectors pANT69 (IgG1 (H310A, H435Q)) and pANT70 (IgG4 (S228P, L235E, H310A, H435Q)) as outlined above.

Chimeric (VH0/Vκ0), VH3/Vκ4, VH4/Vκ3 and VH4/Vκ4 were expressed as IgG1, IgG1 (H310A, H435Q), and IgG4 (S228, L235E, H310A, H435Q) (12 combinations in total) following transient transfection of FreeStyle™ CHO-S cells (ThermoFisher, Loughborough, UK) with corresponding plasmids and using a MaxCyte STX® electroporation system (MaxCyte Inc., Gaithersburg, USA. Following cell recovery, cells were diluted to 3×106 cells/ml in CD Opti-CHO medium (ThermoFisher, Loughborough, UK) containing 8 mM L-Glutamine (ThermoFisher, Loughborough, UK) and 1× Hypoxanthine-Thymidine (ThermoFisher, Loughborough, UK). 24 hours post-transfection, the culture temperature was reduced to 32° C. and 1 mM sodium butyrate (Sigma, Dorset, UK) was added. Cultures were fed on day 1 with 30% CD Efficient Feed B (ThermoFisher, Loughborough, UK) and 3.3% FunctionMAX™ TiterEnhancer (ThermoFisher, Loughborough, UK) and again on day 7 with 15% CD Efficient Feed B (ThermoFisher, Loughborough, UK) and 1.65% FunctionMAX™ TiterEnhancer (ThermoFisher, Loughborough, UK) (Percentages based on starting culture volumes). IgG supernatant titres were monitored by IgG ELISA and transfected cells were cultured for up to 14 days prior to harvesting supernatants.

### Purification of Chimeric and Lead Humanised Antibodies Expressed as Different IgGs

The chimeric (VH0/Vκ0), VH3/Vκ4, VH4/Vκ3 and VH4/Vκ4 IgG1 antibodies and VH4/Vκ4 IgG4 (S228P, L235E) antibody were purified from cell culture supernatants using Protein A sepharose columns. Chimeric (VH0/Vκ0), VH3/Vκ4, VH4/Vκ3 and VH4/Vκ4 expressed as IgG1 (H310A, H435Q) and IgG4 (S228P, L325E, H310A, H435Q) were purified using Protein G columns. The purified antibodies were processed as outlined above. Antibodies were analysed by reducing SDS-PAGE of 2 μg loaded antibody and bands corresponding to the profile of a typical IgG were observed.

### Multicycle Kinetics Analysis of Chimeric Antibodies and Lead Humanised Antibodies Expressed as Different IgGs

Multi-cycle kinetics analysis was performed on the chimeric (VH0/Vκ0), VH3/Vκ4, VH4/Vκ3 and VH4/Vκ4 antibodies on the different IgG backbones using a Biacore T200 (serial no. 1909913) instrument running Biacore T200 Evaluation Software V3.0.1 (Uppsala, Sweden) to establish an accurate affinity for carbonic anhydrase IX. The multicycle kinetics was performed as outlined in Section 2.7. VH0/Vκ0 IgG1 was run as a reference on Fc2 of each run. The relative KD was calculated by dividing the KD of the humanised variants by that of the chimeric antibody with the same IgG constant domain. The lead variants, in all three IgG backbones, all bound within two-fold of VH0/Vκ0 with the same IgG backbone.

### Conclusion and Discussion

V region genes from the Girentuximab antibody together with four humanised VH regions and five humanised Vκ regions (designed using Composite Human Antibody™ technology) were cloned into IgG1 (same allotype as Girentuximab) heavy chain and kappa light chain vectors. Chimeric (VH0/Vκ0) was additionally cloned into VH vectors IgG1 (H310A, H435Q) and IgG4 (S228P, L235E, H310A, H435Q) where mutations H310A and H435Q abolish FcRn binding.

Antibodies transiently transfected into HEK293 EBNA cells were tested for binding to CAIX by single cycle Biacore analysis. Six IgG1 lead antibodies (VH3/Vκ2, VH3/Vκ3, VH3/Vκ4, VH4/Vκ2, VH4/Vκ3 and VH4/Vκ4) were identified based on expression levels and single cycle kinetics data. Purified leads, tested by Biacore multicycle analysis and competition ELISA, were shown to bind within two-fold of the IgG1 chimeric antibody.

Based on iTope™ analysis, percentage humanness, multicycle kinetics data and competition ELISA data, the six leads were narrowed down to three; VH3/Vκ4, VH4/Vκ3 and VH4/Vκ4. These three lead variants were re-cloned and antibodies were transiently expressed in CHO-S cells on three different back bones (IgG1, IgG1 (H310A, H435Q) and IgG4 (S228P, L235E, H310A, H435Q). All antibodies were purified and then tested by Biacore multicycle kinetic analysis. The lead humanised variants, in all three IgG backbones, all showed binding within two-fold of VH0/Vκ0 with the same IgG backbone.

### Example 2: Antibodies for Binding to PSMA

### Summary

The antibody VH gene sequences for the two antibodies, ANT4044 and ANT4044-A2, were cloned into three different human IgG dual expression vectors encoding unmodified IgG1, IgG1 harbouring the mutations H310A and H435Q (that abolish FcRn binding and Protein A binding (Andersen, et al., 2012)) (referred to as IgG1 (H310A, H435Q)) and a modified IgG4 with the same FcRn abolishing mutations described above, together with the hinge stabilising S228P mutation (Angal, et al., 1993) and the Fc silencing L235E mutation (Reddy, et al., 2000) (referred to as IgG4 (S228P, L235E, H310A, H435Q)). Each dual expression vector also contained the antibody Vκ gene sequence common to both ANT4044 and ANT4044-A2.

A total of five antibodies were transiently transfected and expressed in CHO cells and purified using either Protein A (ANT4044-A2 IgG1) or Protein G (both ANT4044 and ANT4044-A2 as both IgG1 (H310A, H435Q) and IgG4 (S228P, L235E, H310A, H435Q)). Affinity chromatography was followed by preparative size exclusion chromatography (SEC).

Antibody integrity was assessed by SDS-PAGE, analytical SEC, thermal stability and antigen binding to PSMA by Biacore. Additional testing was conducted against a panel of human Fc gamma receptors (FcγRIIIA176F, FcγRIIIA176V, FcγRIIIB, FcγRIIA167R, FcγRIIA167H, FcγRIIB, FcgRI) as well as the neonatal receptor, FcRn, using Biacore single cycle analysis.

### Methods and Results

### Construction of Antibody-Expression Plasmids

The VH and Vκ sequences of the humanised antibody ANT4044 and the affinity matured, humanised antibody ANT4044-A2 were used to generate DNA fragments with flanking restriction enzyme sites for cloning into the pANT dual expression vector for IgG1 (pANT18), IgG1 (H310A, H435Q) (pANT71) and IgG4 (S228P, L235E, H310A, H435Q) (pANT73). The VH regions were cloned between the Mlu I and Hind III restriction sites, and the Vκ regions were cloned between the Pte I and BamH I restriction sites within each isotype vector. All five constructs were confirmed by DNA sequencing.

### Transient Expression of Antibodies

Endotoxin-free DNA corresponding to the five antibody constructs was prepared and transiently transfected into CHO-S cells (ThermoFisher, Loughborough, UK) using a MaxCyte STX® electroporation system (MaxCyte Inc., Gaithersburg, USA). Following recovery, cells were diluted to 3×10⁶ cells/ml in CD OptiCHO medium (ThermoFisher, Loughborough, UK) containing 8 mM L-Glutamine (ThermoFisher, Loughborough, UK) and 1× Hypoxanthine-Thymidine (ThermoFisher, Loughborough, UK). 24 hours post-transfection, the culture temperature was reduced to 32° C. and 1 mM sodium butyrate (Sigma, Dorset, UK) was added.

Cultures were fed daily by the addition of 3.6% (of the starting volume) feed (2.5% CHO CD Efficient Feed A (ThermoFisher, Loughborough, UK), 0.5% Yeastolate (BD Biosciences, Oxford, UK), 0.25 mM Glutamax (ThermoFisher, Loughborough, UK) and 2 g/L Glucose (Sigma, Dorset, UK)). IgG supernatant titres were monitored by IgG ELISA and transfected cells were cultured for up to 14 days prior to harvesting supernatants.

### Antibody Purification

Cell culture supernatants were passed over either Protein A (ANT4044-A2 IgG1) or Protein G (both ANT4044 and ANT4044-A2 as both IgG1 (H310A, H435Q) and IgG4 (S228P, L235E, H310A, H435Q)) Sepharose columns (GE Healthcare, Little Chalfont, UK). All antibodies were buffer exchanged into 1×PBS, pH 7.2. Protein A or Protein G purified material was run on a HiLoad™ 26/600 Superdex™ 200 μg preparative SEC column (GE Healthcare, Little Chalfont, UK) using 1×PBS as mobile phase during which monomeric fractions were collected, pooled and filter sterilised. SEC profiles revealed that antibodies, particularly ANT4044-A2, when expressed as IgG4 (S228P, L235E, H310A, H435Q) showed greater levels of aggregation (up to 22%) as compared with antibodies expressed as other IgG isotypes.

Antibodies were quantified by measuring the OD280 nm and using extinction coefficients (Ec(0.1%)) based on their predicted amino acid sequences.

### Analytical SEC and SDS-PAGE

Stock ANT4044 IgG1 and Protein A or Protein G followed by preparative SEC purified material was analysed by analytical SEC using a Superdex™ 200 Increase 10/300 GL analytical column (GE Healthcare, Little Chalfont, UK) and 1×PBS as mobile phase. The elution profiles were typical for correctly folded monomeric species of IgG. Antibodies were also analysed by non-reducing and reducing SDS-PAGE. Bands corresponding to the predicted sizes of VH and Vκ chains were observed

### Thermostability Analysis

To assess the thermostability of the five purified antibodies together with ANT4044 IgG1 from stock, melting temperatures (the temperature at which 50% of a protein domain is unfolded) were determined using a fluorescence-based thermal shift assay. All antibodies were diluted to a working concentration of 100 μg/ml in 1×PBS containing SYPRO® Orange (ThermoFisher, Loughborough, UK) and subjected to a temperature gradient from 25° C. to 99° C. on a StepOnePlus real-time PCR system (ThermoFisher, Loughborough, UK) over a period of 56 minutes. The melting curves were analysed using protein thermostability software (version 1.2) and Tms were calculated based on first derivative data.

For both ANT4044 and ANT4044-A2, the IgG1 backbone appeared the most thermally stable with the lowest melting temperatures for both being 69.3° C. When comparing the different IgG backbones, ANT4044 exhibited greater thermal stability as compared with ANT4044-A2.

### Assessment of Binding to PSMA

Multi-cycle kinetic analysis was performed on each of the purified antibodies in order to assess binding to prostate-specific membrane antigen (PSMA). The analysis was performed using a Biacore T200 (serial no. 1909913) instrument running Biacore T200 Evaluation Software V3.0.1 (Uppsala, Sweden. For direct comparison, all antibodies were captured on a Protein G chip (GE Healthcare, Uppsala, Sweden).

Purified antibodies were diluted to a concentration of 1 μg/ml in HBS-EP+. At the start of each cycle, each antibody was captured on the Protein G surface to give an RL of ˜50 RU. Following capture, the surface was allowed to stabilise. Kinetic data was obtained using a flow rate of 35 μl/min to minimise any potential mass transfer effects. For the kinetic analysis, PSMA (R&D Systems, Minneapolis, U.S.A) was used. Multiple repeats of the blank (PSMA) and a repeat of a single concentration of the analyte were programmed into the kinetic run to check the stability of both the surface and analyte over the kinetic cycles. For kinetic analysis, a twofold dilution range was selected from 25 nM to 1.5625 nM PSMA. The association phase of PSMA was monitored for 600 seconds and the dissociation phase was monitored for 2400 seconds. Regeneration of the Protein G surface was conducted using two injections of 10 mM glycine-HCL pH 1.5 containing 0.5% P20 at the end of each cycle.

The signal from the reference channel Fc1 was subtracted from that of Fc2, Fc3 and Fc4 to correct for differences in non-specific binding to a reference surface, and a global Rmax parameter was used in the 1-to-1 binding model. The relative KD was calculated by dividing the KD of each antibody by that of ANT4044 IgG1 on the same chip.

### Assessment of Binding to Human FcRn

The binding of the purified antibodies to FcRn was assessed by steady state affinity analysis using a Biacore T200 (serial no. 1909913) instrument running Biacore T200 Evaluation Software V3.0.1 (Uppsala, Sweden). FcRn (Sino Biological, Beijing, China) was coated onto a CM5 chip at 10 μg/mL in sodium acetate pH 5.5 using standard amine coupling to 300 RU.

Purified antibodies were titrated in a five-point dilution from 37 nM to 3000 nM in PBS containing 0.05% P20 at either pH 6.0 or pH7.4. Antibodies were passed over the chip in increasing concentrations at a flow rate of 30 μl/min and at 25° C. The injection time was 30 s and the dissociation time was 100 s. Following a single dissociation, the chip was regenerated with 0.1M Tris pH 8.0.

As expected, the H310A H435Q mutations significantly reduced antibody binding to FcRn at pH 6.0. Both ANT4044 and ANT4044-A2 antibodies tested as unmodified IgG1 showed similar affinities toward FcRn at pH 6.0. Very little binding was observed for any of the antibodies at pH 7.4.

### Assessment of Binding to Human Fc Gamma Receptors

Binding of purified antibodies to high and low affinity Fc gamma receptors was assessed by single cycle analysis using a Biacore T200 (serial no. 1909913) instrument running Biacore T200 Evaluation Software V3.0.1 (Uppsala, Sweden) running at a flow rate of 30 μl/min. The human Fc receptors, FcγRI, FcγRIIa (both 167R and 167H polymorphisms), FcγRIIb, FcγRIIIa (both 176F and 176V polymorphisms) and FcγRIIIb were obtained from Sino Biological (Beijing, China). FcγR were captured on a CM5 sensor chip pre-coupled using a Hiscapture kit (GE Healthcare, Little Chalfont, UK) using standard amine chemistry.

At the start of each cycle His-tagged Fc gamma receptors diluted in HBS-P+ were loaded to a specific RU level. A five point, three-fold dilution range of antibody without regeneration between each concentration was used for each receptor. In all cases, following dissociation the chip was regenerated with two injections of Glycine pH 1.5. The signal from the reference channel Fc1 (blank) was subtracted from that of the Fc loaded with receptor to correct for differences in non-specific binding to the reference surface.

Sensorgrams were analysed for 1:1 kinetics for the high affinity Fc gamma receptor FcγRI and by steady state binding for the low affinity Fc gamma receptors.

### Representative Sensorgrams

ANT4044 and ANT4044-A2 expressed as unmodified IgG1 bound to all high affinity and low affinity human activating Fcγ receptors. Introduction of the H310A and H435Q mutations in IgG1 did not affect this binding although a small trend towards slightly lower binding to low affinity human Fcγ receptors was observed. As anticipated, the IgG4 (S228P, L235E, H310A, H435Q) antibodies showed markedly reduced binding to all activating Fcγ receptors. All antibodies tested showed low affinity binding to the inhibitory receptor FcγRIIB. Thus, both IgG1 and IgG1 (H310A, H435Q) are potentially able to stimulate effector function whereas IgG4 (S228P, L235E, H310A, H435Q) is unlikely to stimulate effector function.

### Conclusion

The variable heavy and light chain sequences corresponding to the humanised anti-PSMA antibody ANT4044 and its affinity matured variant ANT4044-A2 were cloned into dual expression vectors encoding either unmodified human IgG1, human IgG1 with the mutations H310A and H435Q (which abolish FcRn binding), or human IgG4 (S228P, L235E, H310A, H435Q). CHO cells were transiently transfected and five antibodies purified by Protein A or Protein G affinity chromatography and preparative SEC. Analytical SEC revealed profiles that were consistent with monomeric IgG with little evidence of aggregation. All antibodies including ANT4044 IgG1 (from stock) were characterised in terms of their thermostability and binding to PSMA, human FcRn and human Fcγ receptors.

For both ANT4044 and ANT4044-A2, the IgG1 backbone appeared the most thermally stable while ANT4044 antibodies exhibited greater thermal stability as compared with ANT4044-A2 antibodies. All antibodies in both a IgG1 (H310A, H435Q) and a IgG4 (S228P, L235E, H310A, H435Q) format showed similar binding to PSMA as their counterparts expressed as unmodified IgG1. Affinity matured ANT4044-A2 antibodies showed a 2-3-fold greater affinity for PSMA as compared with ANT4044.

Binding to FcRn at pH 6.0 was significantly reduced by the introduction of the H310A, H435Q mutations while very little binding to FcRn was observed for any of the antibodies at pH 7.4.

Analysis of antibody binding to human Fcγ receptors confirmed the abolition of binding by the L235E mutation in antibodies expressed as IgG4 (S228P, L235E, H310A, H435Q). The mutations H310A and H435Q did not significantly impact the binding of antibodies to human Fcγ receptors and thus, both IgG1 and IgG1 (H310A, H435Q) are anticipated to show similar effector function properties.

### Example 3: Conjugation of Antibodies

Antibodies ANT4044-IgG1, ANT4044-A2-IgG1, ANT4044-IgG1 H310A H435Q (a.k.a. ANT4044-IgG1-2M) and ANT4044-IgG4 S228P L235E H310A H435Q (a.k.a. ANT4044-IgG4-4M) were conjugated to either a ThioBridge™-PEG(6u)-DOTA reagent or an NHS-DOTA reagent.

ThioBridge™ is PolyTherics' proprietary disulfide conjugation linker and is described in

DOTA is a chelator payload, 1,4,7,10-tetraazacyclododecane-1,4,7,10-tetraacetic acid mono amide.

ThioBridge™ DOTA conjugation evaluation: ANT4044-IgG1 was prepared as a 6-10 mg/mL solution in reaction buffer (20 mM Sodium Phosphate, pH 7.5, 150 mM NaCl, 20 mM ethylenediaminetetraacetic acid (EDTA). To ANT4044-IgG1 in reaction buffer (6-10 mg/mL, 40° C.) was added 6-10 equivalents of tris(2-carboxyethyl)phosphine (TCEP) per antibody or DTT 10 mM. The antibody concentration was adjusted to 5 mg/mL by dilution with reaction buffer. The reduction mixture was incubated for 1 hour at 37-40° C. The reduction mixture was cooled down to 22° C. prior to addition of reagent. 5.6-8 equivalents of ThioBridge™-PEG(6u)-DOTA in acetonitrile were added to the mixture, which was further diluted to 4 mg/mL with reaction buffer. Percentage of acetonitrile in the mixture was 5%. The reaction mixture was incubated for up to 22 h at 22° C. Buffer exchange and excess reagent removal was carried out by ultracentrifugation at 14,000 rcf with Vivaspin 20 filters (30 kDa MWCO, PES membrane, Generon). The sample was buffer exchanged 7-9× into Dulbecco's PBS pH 7.2-7.5 with Vivaspin 20 filters (30 kDa MWCO, PES membrane, Generon). Antibody-ThioBridge™ DOTA conjugate concentration was measured by UV-vis, corrected to 4.0 mg/mL with Dulbecco's-PBS pH 7.2-7.5, sterile filtered (0.22 μm cellulose acetate filters) and stored at −80° C.

Lysine-DOTA conjugation evaluation: ANT4044-IgG1 was prepared as a 6 mg/mL solution in 0.1 M NaHCO₃ and 20 mM ethylenediaminetetraacetic acid (EDTA), pH 8-9 (reaction buffer). Next, prepared 1,4,7,10-Tetraazacyclododecane-1,4,7,10-tetraacetic acid mono-N-hydroxysuccinimide ester hexafluorophosphate trifluoroacetate salt (NHS-DOTA reagent) in Dulbecco's PBS pH 7.2-7.5 at 5.0 mg/mL. Added 10-25 equivalents of NHS-DOTA reagent solution and corrected antibody concentration to 4.0 mg/mL by addition of reaction buffer. Incubated at 22° C. for 2-3 h. Next, quenched by addition of 0.2 M sodium acetate, pH 5.5 (4:1 v/v) and ultracentrifuged at 14,000 rcf with Vivaspin 20 filters (30 kDa MWCO, PES membrane, Generon). Repeated dilution and ultracentrifugation 2× more. Then, buffer exchanged 4× into Dulbecco's PBS pH 7.2-7.5 with Vivaspin 20 filters (30 kDa MWCO, PES membrane, Generon). Antibody-DOTA conjugate concentration was measured by UV-vis, corrected to 4.0 mg/mL with Dulbecco's-PBS pH 7.2-7.5, sterile filtered (0.22 μm cellulose acetate filters) and stored at −80° C. Small-scale reactions and purifications were first carried out to identify appropriate conjugation conditions. Analytical SEC and analytical LC-MS methods were developed to confirm extent of conjugation, purity and residual reagent present. It was found that conjugation was efficient for both reagents. Neither reagent resulted in aggregation during or after conjugation. Lysine conjugates displayed a wider range of differently DOTA loaded species and required higher amounts for LC-MS analysis than ThioBridge™ conjugates. All samples tested were shown to have an average DAR between 4.0 and 4.2 (ThioBridge™ conjugates) and average DAR between 3.8 and 4.9 (Lysine conjugates) by LC-MS.

### Example 4 Pharmacokinetic Analysis of Antibodies Binding to CAIX

The pharmacokinetic (PK) characteristics of various antibodies of the invention, were determined in a mouse model, B6.Cg-Fcgrt^{tm1Dcr} Tg(FCGRT)32Dcr/DcrJ.

B6.Cg-Fcgrt^{tm1Dcr} Tg(FCGRT)32Dcr/DcrJ mice (Tg32) are humanized for FcRn (hFcRn) expression by means of their transgene being driven by the human endogenous FcRn promoter. Published tissue FcRn expression and human IgG PK data closely mimic human patient data, with higher correlation to human data than data obtained from what has served as the gold standard for human IgG PK analysis, the non-human primate. The Tg32 mice utilized in this PK study are hemizygous for the Tg(FCGRT)32Dcr transgene to allow antibody half-life to be suboptimally maximized in the intermediate range allowing antibody-hFcRn affinity differences to be measured.

The pharmacokinetics of the following antibodies was determined: chimeric girentuximab (ChGmAb), humanised girentuximab (HuGmAb), humanised girentuximab comprising amino acid substitutions in the FcRn-binding domain (HuGmAb-FcRn), and humanised girentuximab comprising amino acid substitutions in the FcRn binding and the Fc gamma receptor binding domains (HuGmAb-FcRg).

### Methods

Each of the antibodies was administered intravenously into Tg32 hemizygous mice at 5 mg/kg in a volume of 5 ml/kg.
a) In Vivo Study Design
i) Tg32 mice will be distributed into 4 groups with 6 mice per group, as outlined in Table 1.
ii) All mice were weighed and 25 μL blood samples were collected with K3EDTA as an anticoagulant 1 day prior to compound administration at time 0 to act as a pre-bleed. Blood was collected from the retro-orbital sinus using small diameter capillary tubes (Drummond #1-000-0250) and blood samples were processed to plasma. Ten microliters of plasma samples were diluted 1/10 with Storage Buffer (50% glycerol in PBS), flash frozen in specialized 96 well storage plates, and stored at −20° C.
iii) At 0 hours, test articles were administered to Tg32 mice as IV injections at 5 mg/kg in dose volumes of 5 ml/kg.
iv) Blood samples were collected from each mouse according to a bleeding schedule at 30 minutes, as well as 1, 2, 3, 5, 7, 10, and 14 days. The blood samples were processed to plasma and stored as described above.
v) At the end of the 14 day in vivo experiment, study samples were assessed by ELISA.

### Results

FIG. 1A shows the serum half lives for the antibodies tested. The results clearly show a significant reduction in antibody half life for antibodies having amino acid substitutions in the FcRn binding site or in the FcRn and Fc gamma receptor binding sites.

### Example 5: Pharmacokinetic Analysis of Antibodies for Binding to PSMA

The serum half lives of the following antibodies was assessed using a similar methodology to that described in Example 1: J591 IgG lysine DOTA conjugate (control antibody for binding PSMA), ANT4044 lysine DOTA conjugate (ANT4044-K-DOTA), ANT4044-A2 lysine DOTA conjugate (ANT4044-A2-K-DOTA), ANT4044 with amino acid substitutions in the FcRn-binding region, lysine DOTA conjugate (ANT4044-FcRn-K-DOTA) and ANT4044 with amino acid substitutions in the FcRn and Fc gamma receptor binding regions, lysine DOTA conjugate (ANT4044-FcRg-K-DOTA). The results are shown in FIG. 1B.

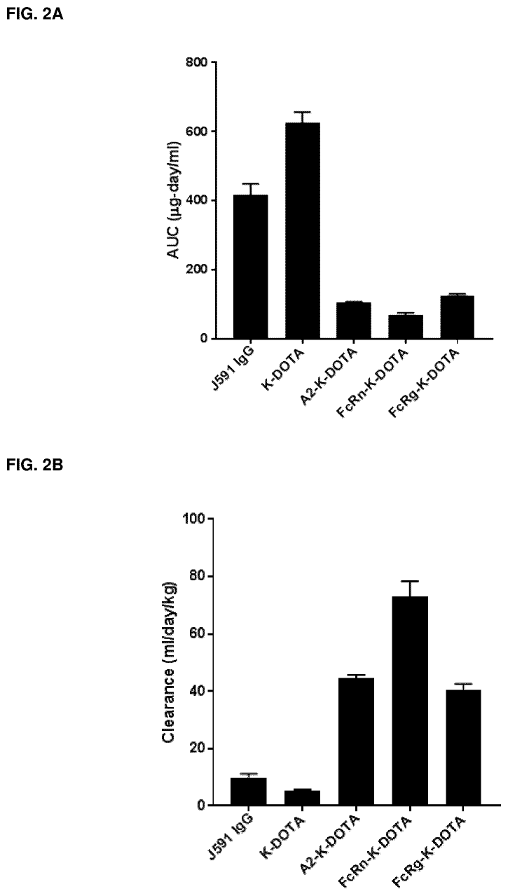

FIG. 2 shows the average area under the curve (AUC, top) and clearance (CL, bottom) for each test antibody. Error bars represent standard error of the mean.

### Example 6: Biodistribution and Tumour Accumulation Studies

Quantitative analysis of the comparative targeting of different antibodies of the invention to LNCaP cells in mouse models was assessed.

### Radiolabelling and TLC Analysis of Antibodies.

The test antibodies were:
1. ANT4044-IgG1+DOTA (“JN005”)
2. ANT4044-A2-IgG1+DOTA (“JN008”)
3. ANT4044-IgG1(FcRn)+DOTA (“JN007”)
4. ANT4044-IgG4(FcRn/FcRgamma)+DOTA (“JN006”)
5. HuJ591 IgG1+DOTA (“J591”—control for binding to PSMA)

All antibodies were incubated with ⁶⁴Cu at a 200-fold excess of biomolecule in 0.1 M pH 5.5 Ammonium Acetate buffer for 45 minutes at room temperature. Samples of each solution were taken and mixed 1:1 with 50 mM EDTA. 5 μL of each solution was spotted on TLC paper (Agilent iTLC-SG Glass microfiber chromatography paper impregnated with silica gel) and run with 50:50 H₂O:Ethanol. Plates were then imaged on a Carestream MSFX imaging system using a radioisotopic phosphor screen. Where necessary, unbound copper was removed by purification using 7 K MWCO Zeba Spin Columns (Thermo Scientific) as per manufacturers protocols. All samples showed >95% labelling.

Control experiments were conducted to monitor the elution behaviour of free Cu-64 and Cu-64 bound to EDTA for quality control.

### Tumour Initiation and Growth

8 week old male Balb/c nude mice were injected (27G needle) subcutaneously with 5×10\hat{ }6 LNCaP cells in 100 μL phosphate buffered saline into the right flank of each mouse.

Antibody solutions were injected via the tail vein (29G) and then mice were imaged using the Siemens Inveon PET-CT instrument, or blood collected via tail snip and activity measured via gamma counter for blood concentration analysis at indicated timepoints.

### Imaging Protocol

Mice were anaesthetised with isoflurane (IsoFlo, Abbott Laboratories) at a dose of 2% in a closed anaesthetic induction chamber. Mice were monitored using ocular and pedal reflexes to ensure deep anaesthesia. Once the mouse was deeply anesthetised, it was placed on an appropriate animal bed, where the anaesthetic air mixture (1%) was delivered to its nose and mouth through a nose cone. Physiological monitoring (respiratory using a sensor probe) was achieved throughout all experiments using an animal monitoring system (the BioVet™ system, m2m Imaging, Australia). Images were acquired using a Siemens Inveon PET-CT scanner following tail vein intravenous injection of the antibodies.

The injection syringe was filled with the radioisotope solution (approximately 150 μL) and the activity in the syringe was measured using a dose calibrator (Capintec CRC-25) with a calibration factor of 35. The activity left in the syringe after the tail vein injection was measured using the same dose calibrator and the total volume injected in each mouse was calculated.

Calibration of the PET/CT scanner was performed with an in-house manufactured phantom containing a dose of Cu-64 solution as a radiation source.

The mice were positioned on the scanner bed (n=4 per scan using a bed developed in-house) and micro-CT scans were acquired for anatomical co-registration. The CT images of the mice were acquired through an X-ray source with the voltage set to 80 kV and the current set to 500 pA. The scans were performed using 360° rotation with 120 rotation steps with a low magnification and a binning factor of four. The exposure time was 230 ms with an effective pixel size of 106 μm. The CT images were reconstructed using Feldkamp reconstruction software (Siemens). Following CT imaging, PET scans were acquired at, 8 hours, 24 hours and 48 hours after injection of the radiotracer, using 30-60 minute static acquisitions. The PET Images were reconstructed using an ordered-subset expectation maximisation (OSEM2D) algorithm and analysed using the Inveon Research Workplace software (IRW 4.1) (Siemens) which allows fusion of CT and PET images and definition of regions of interest (ROIs). CT and PET datasets of each individual animal were aligned using IRW software (Siemens) to ensure good overlap of the organs of interest. Three dimensional ROIs were placed within the whole body, as well as all the organs of interest, such as heart, kidney, lungs, bladder, liver, spleen, intestines and tumour, using morphologic CT information to delineate organs. Activity per voxel was converted to nci/cc using a conversion factor obtained by scanning a cylindrical phantom filled with a known activity of Cu-64 to account for PET scanner efficiency. Activity concentrations were then expressed as percent of the decay-corrected injected activity per cm³ of tissue that can be approximate as percentage injected dose/g (% ID/g).

The tumour to blood ratio was then calculated as the activity detected in tumour relative to the activity detected in blood.

### Results

Mice were imaged at 8, 24 and 48 hrs post-injection. Following the 48 hr timepoint, organs were harvested for gamma counting and quantification of organ distribution.

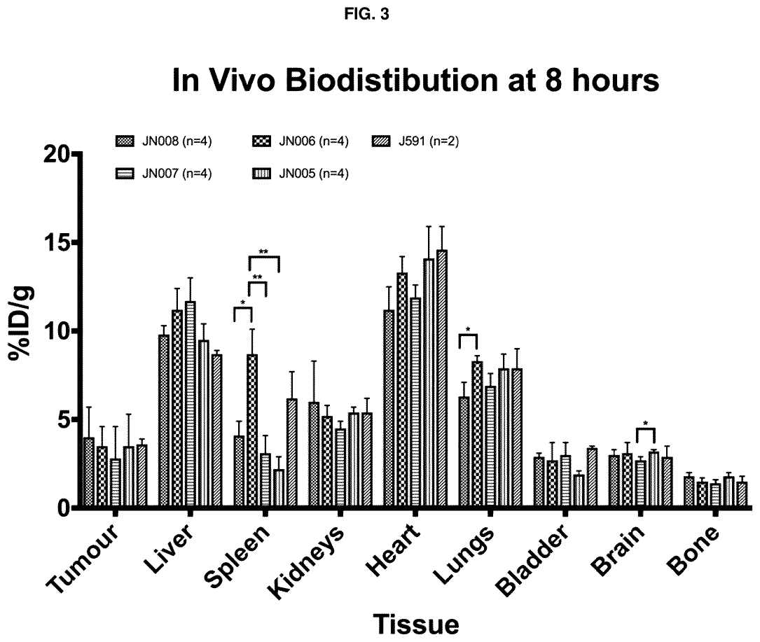

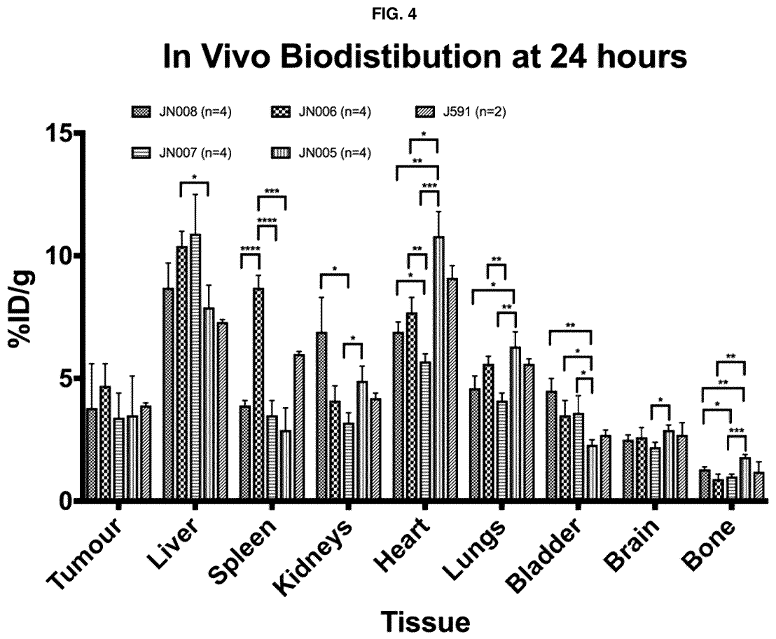

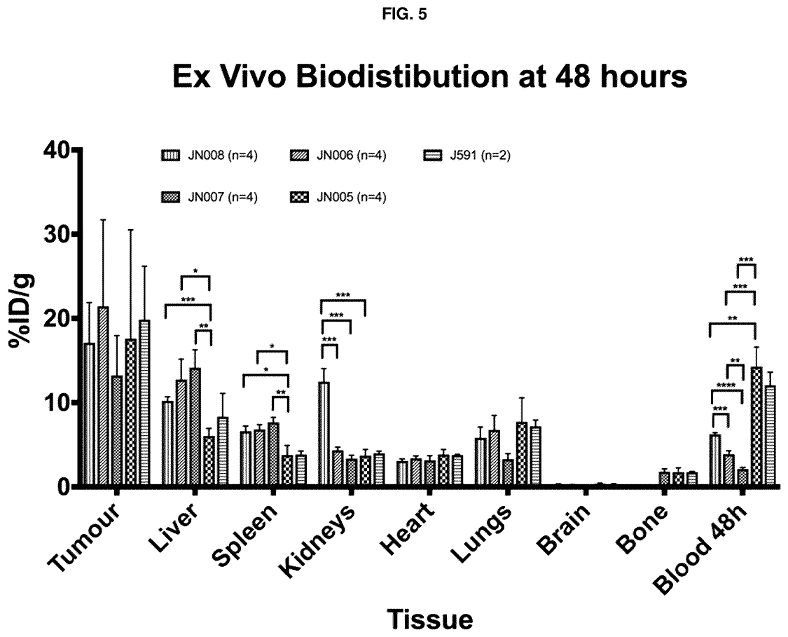

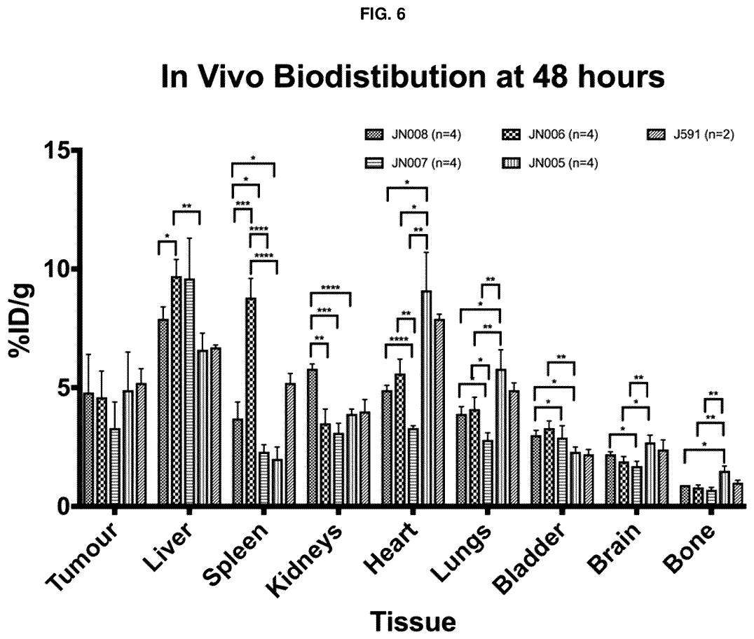

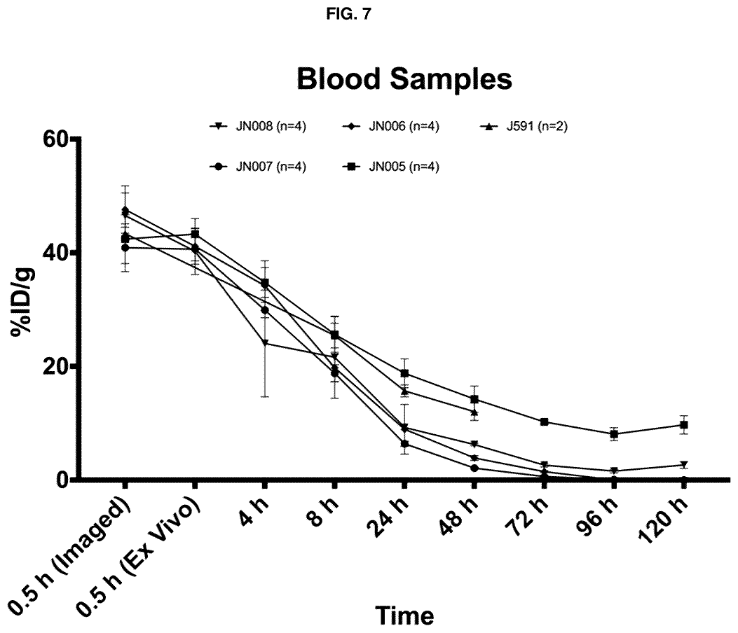

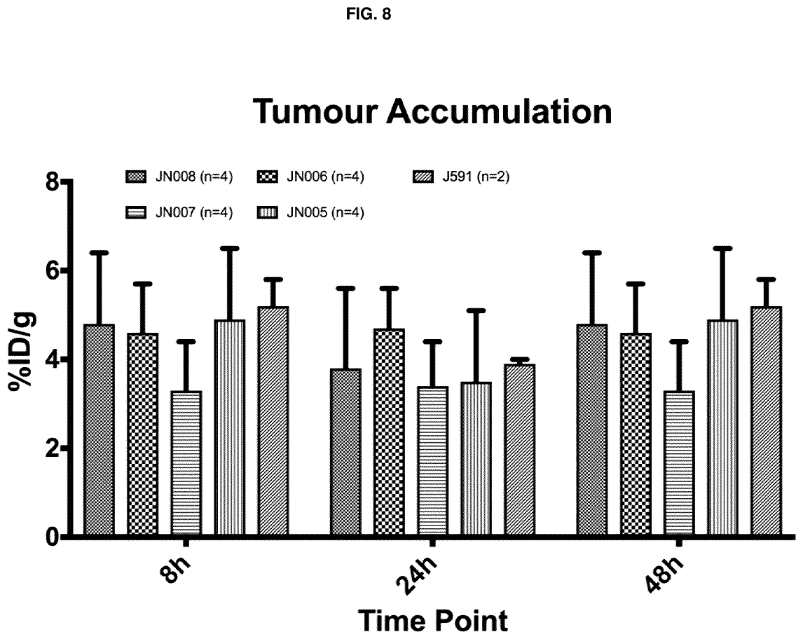

Regions of interest were drawn around the tumour margins (as delineated from the CT scan) and the concentration of antibody calculated for each mouse in the imaging study (based on % injected dose). FIGS. 3 to 6, and 8 show organ accumulation as measured in vivo and ex vivo (by gamma counter). Variability in quantitation between in vivo and ex vivo arises due to ROI and background signal for the in vivo plots. ns P>0.05, * P≤0.05, ** P≤0.01, *** P≤0.001, **** P 0.0001 FIG. 7 shows blood concentration of antibodies out to 5 days post-injection.

In vivo imaging showed tumour accumulation and long term localisation (over 2 days) for all antibodies. There was no statistical difference between the different antibodies and the amount in the tumours was approximately 5% ID/g out to 48 hrs.

Ex vivo analyses showed biodistribution at 48 hrs for all antibodies. At this timepoint, the tumour accumulation had the highest concentration of antibody, followed by the liver, spleen and lungs.

JN005 shows lower liver and spleen accumulation than the other variants (except J591). This is also evident in the much longer circulation time—there is still ˜10% ID/g of JN005 circulating at 120 hrs.

Pharmacokinetic evaluation was undertaken by taking blood samples out to 120 hrs. The most notable observation was the faster clearance of JN007 compared to the other antibodies.

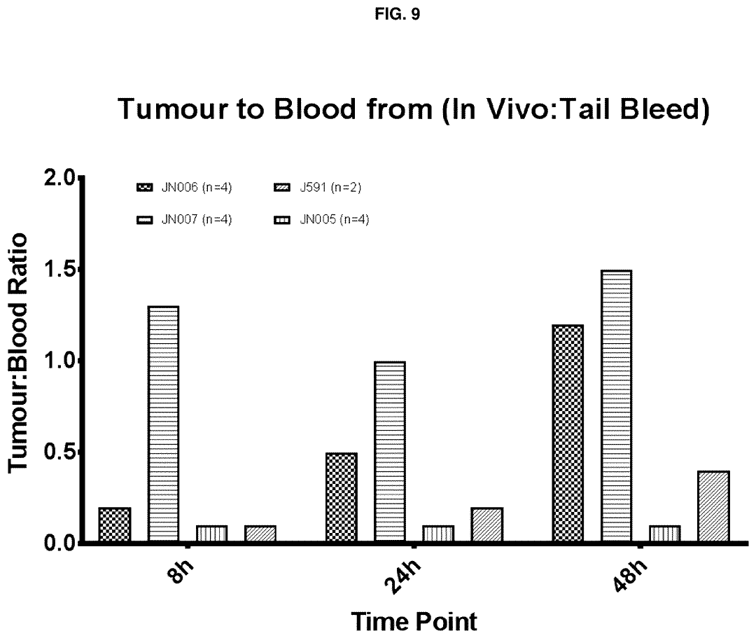

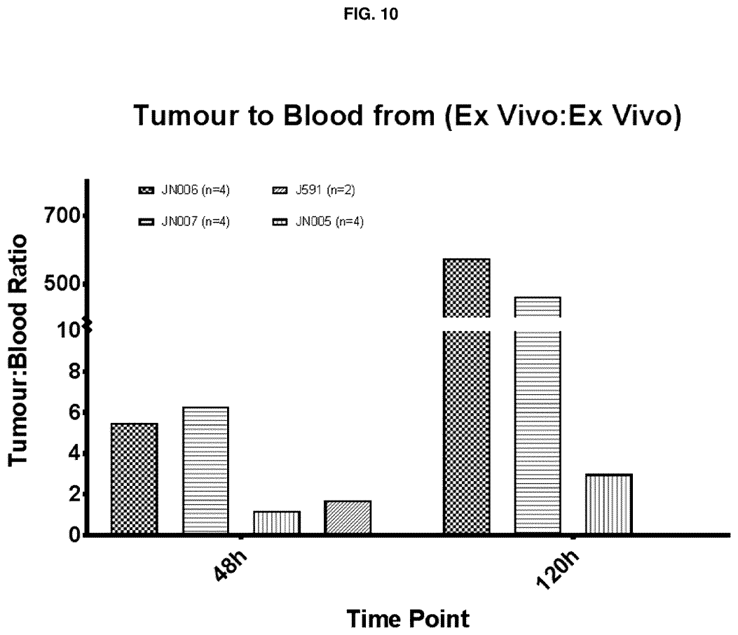

Blood samples were also used to calculate the tumour:blood ratios of the antibodies. The tumour:blood ratio (in vivo:tail bleed) for each of the antibodies was determined for the 8 hr, 24 hr and 48 hr time points and the results are shown in FIG. 9. FIG. 10 shows the tumour:blood ratio (ex vivo:ex vivo) at 48 hrs and 120 hrs.

The tumour:blood ratio is significantly higher for antibodies JN006 (ANT4044-IgG4(FcRn/FcRgamma)+DOTA) and JN007 (ANT4044-IgG1(FcRn)+DOTA) at all time points compared with antibodies JN005 (ANT4044-IgG1+DOTA) and J591. The ratio at 120 hr is particularly striking, with the tumour:blood ratio for FcRn-binding modified and for FcRn/Rf gamma receptor-binding modified antibodies exceeding that of non-modified antibodies by approximately 200-fold.

### Discussion

Despite some of the tested antibodies having significantly reduced serum half-lives (and increased clearance), the amount of all antibodies accumulating in the tumours was not statistically different. These results are surprising in that they show that tumour loading for each of the antibodies was the same, indicating that all antibodies had similar binding affinities for their target epitopes, and all antibodies had a similar capacity to be delivered to the target sites, even though the FcRn and Fc gamma receptor modifications significantly increased clearance and reduced the serum half life of the JN007 and JN006 antibodies.

The results indicate that modification of the FcRn or the FcRn and Fc gamma receptor binding domains of radiolabelled antibodies has significant utility in reducing the amount of radioisotope in the circulation, without impacting on the therapeutic potential of the antibody with respect to its capacity to accumulate in the tumour. This has numerous benefits, including reducing a number of toxic effects which would otherwise result from longer-term residence of radioisotope in the circulation (including haematological toxicity, absorption into bone and bone marrow irradiation).

### Example 7: ¹⁷⁷Lu Imaging and Radiotherapy Efficacy Study of an Exemplary Antibody of the Invention

### Test Articles

1.
TXP02-JN007 (ANT4044-FcRn-K-DOTA)
2. TXP02-JN005 (ANT4044-K-DOTA)
3. PSMA-617 (PSMA-binding peptide)

### Phase 1 Chemistry: Imaging Study

### Labelling Overview: JN007, JN005.

ANT4044 is an anti-PSMA antibody as described herein. ANT4044-FcRN is a modified form of the antibody, in which the FcRn-binding region of the heavy constant chain has been modified to reduce serum half-life. ANT4044-FcRn-K-DOTA is ANT4044-FcRN, conjugated to the chelator DOTA via lysine residues.

150 μg of ANT4044-FcRn was labelled with 500 MBq of Lu-177 with a 2-hour incubation at 37° C. Labelling efficiency was 81%. The reaction mixture was purified on a NAP-5 column into PBS with a subsequent labelling efficiency of 98%. Fractions containing the labelled antibody were collated. Doses containing 10 μg, 100 μg and 370 μg each labelled with 20 MBq of Lu-177 were prepared by addition of the appropriate amounts of unlabelled ANT4044-FcRn antibody and PBS.

500 μg of ANT4044 antibody was labelled with 100 MBq of Lu-177 with a 2-hour incubation at 37° C. Labelling efficiency was 98.2%. The preparation was diluted in PBS and used without further purification.

### Phase 2 Chemistry: Efficacy Study

### Labelling Overview: PSMA-617, ANT4044-FcRn.

6 μg (4 nmol) of PSMA-617 (6 μl of 1 mg/ml solution in water) was labelled with 200 MBq Lu-177 in 0.1 M ammonium acetate buffer (pH 5.5) and heated to 95° C. for 10 minutes. Labelling efficiency was 97.6%. The preparation was diluted in PBS and used without further purification.

300 μg ANT4044-FcRn was labelled with 120 MBq Lu-177. Labelling efficiency after 2 hours incubation at 37° C. was 96.5%. The preparation was quenched by addition of 5 μl 0.1 M EDTA, diluted in PBS and used without further purification.

### Phase 1—Imaging Study Design

1. For the phase 1 study, animals with the appropriate tumour size (150-300 mm³) were placed on study into 4 groups of n=3 with dosing as follows
   a. Group 1: 10 μg [¹⁷⁷Lu]TXP02-ANT4044-FcRn in 200 μL, intravenous (IV) injection into the lateral tail vein.
   b. Group 2: 100 μg [¹⁷⁷Lu]TXP02-ANT4044-FcRn in 200 μL, IV injection into the lateral tail vein.
   c. Group 3: 370 μg [¹⁷⁷Lu]TXP02-ANT4044-FcRn in 200 μL, IV injection into the lateral tail vein.
   d. Group 4: 100 μg [¹⁷⁷Lu]TXP02-ANT4044 in 200 μL, IV injection into the lateral tail vein.
2. Antibody biodistribution was assessed at 4, 24, and 48 hours post antibody injection via SPECT/CT imaging:
   a. A whole-body static SPECT image was acquired followed by a whole-body CT for anatomical reference.
   b. SPECT scan duration was 40 minutes, CT scan duration was 12 minutes per scan.
   c. Animals were imaged three at a time in a multi-mouse hotel
3. Tumour volume was assessed via calliper measurements 3 times a week. Animals were inspected for any adverse effects and weighed regularly.
4. For select animals tissues were collected for additional ex vivo analysis.
5. Remaining animals were culled and carcasses discarded.

### Phase 2—Efficacy Study Design

1. For the main efficacy study, animals with the appropriate tumour size (150-300 mm³) were placed on study in 3 groups of n=6 with dosing as follows
   a. Group 1: 50 μg [¹⁷⁷Lu]TXP02-ANT4044-FcRn in 200 μL, IV
   b. Group 2: 600 ng [¹⁷⁷Lu]PSMA-617 in 200 μL, IV
   c. Group 3: 200 μL PBS, IV
2. Tumour size was assessed via calliper measurements 3 times a week for 3 weeks following test agent injections.
3. Animals were inspected for any adverse effects and weighed regularly.
4. Blood samples were taken from animals via tail prick method in groups 1 and 2 at 0.5, 4, 8, 24, 48, 72, 96, 120 hours and assessed via gamma counting.
5. Whole blood samples were weighed and then counted in the gamma counter with reference standards. 6. Initial study aim was to monitor the tumour growth for 5 weeks post-treatment.
This time frame was reduced due to the radiation sickness observed in some of the [¹⁷⁷Lu]TXP02-ANT4044-FcRn mice.
7. For select animals tissues were collected for additional ex vivo analysis.
8. Remaining animals were culled and carcasses discarded.

### Results and Discussion

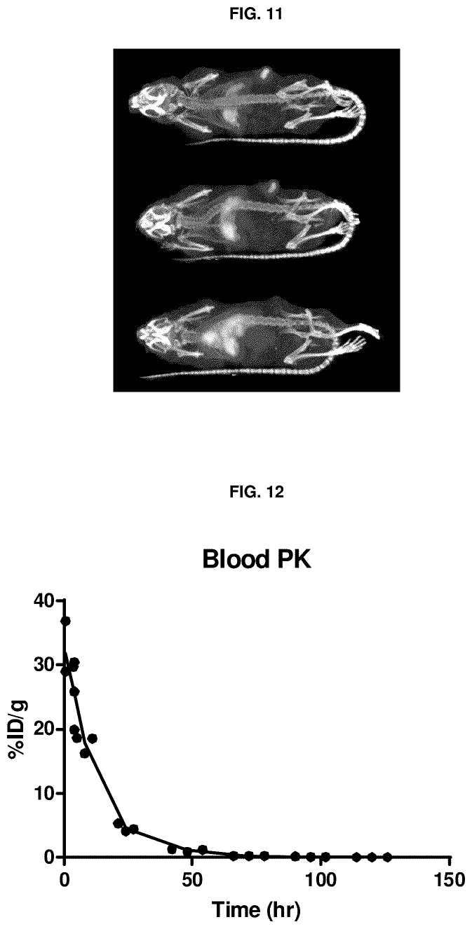

FIG. 11 is an exemplary image of [¹⁷⁷Lu]TXP02-ANT4044-FcRn distribution in the xenograft mice.

FIG. 12 is a plot of [¹⁷⁷Lu]TXP02-ANT4044-FcRn radioactivity levels measured in blood from the mice.

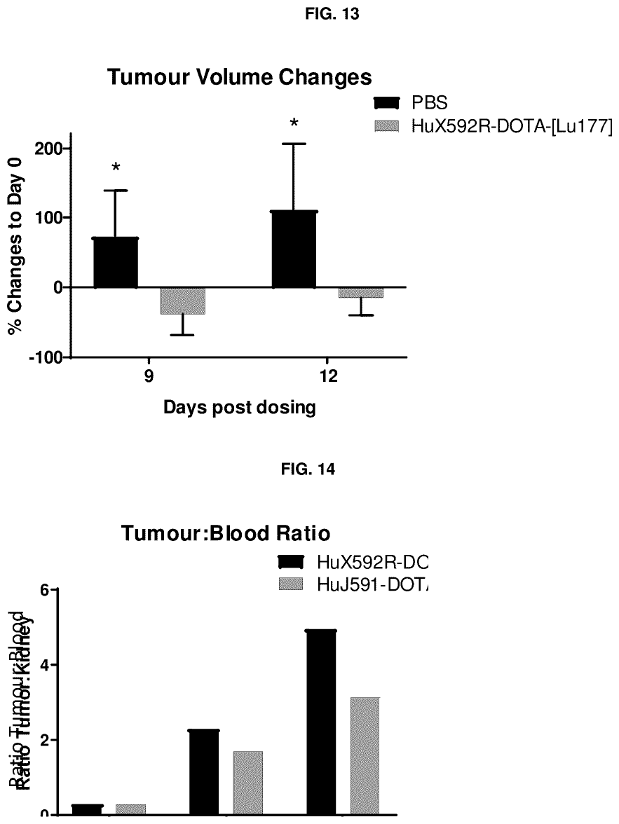

FIG. 13 is a plot of tumour growth as determined in the present study. ANT4044-FcRn-DOTA-Lu treatment significantly suppressed tumour growth as evidence by no change in tumour volume on day 14 as compared to day 0. In the control (PBS) group, there was an overall increase in tumour volume, with tumours becoming significantly larger at 9, 12 and 14 days when compared to the corresponding time in ANT4044-FcRn-DOTA-Lu treated group.

Radiolabelling of both test (JN007) and comparator (JN005) antibodies was successful (>96% labelling efficiency) with a maximum specific activity of 2 MBq/μg achieved.

The imaging study showed uptake of both test and comparator antibodies in the LNCaP tumour xenografts (see Table 3).

Blood clearance, as inferred by a small ROI drawn in the left ventricle of the heart, was faster for the JN007 antibody for all groups at 48 hours post-injection, compared to the same time point for the JN005 antibody (see Table 4).

TABLE 3
[¹⁷⁷Lu] TXP02-JN007 and [¹⁷⁷Lu]TXP02-JN005 Tumour uptake at 4, 24 and 48 hours

| | | | Dose | 4 hrs: | 24 hrs: | 48 hrs: |
|---|---|---|---|---|---|---|
| | | | Radioactivity | Tumour | Tumour | Tumour |
| | Mass dose | | (Mbq/animal, | % ID/g | % ID/g | % ID/g |
| Group | (μg/animal) | Imaging tracer | Mean ± SD) | (Mean ± SD) | (Mean ± SD) | (Mean ± SD) |
| 1 | 100 | [¹⁷⁷Lu] TXP02- | 19.4 ± 0.6 | 3.2 ± 0.4 | 6.9 ± 1.2 | 8.3 ± 1.7 |
| | | JN007 | | | | |
| 2 | 100 | [¹⁷⁷Lu] TXP02- | 19.7 ± 0.7 | 4.5 ± 0.2 | 16.8 ± 1.2 | 23.7 ± 0.8 |
| | | JN005 | | | | |

TABLE 4
[177Lu] TXP02-JN007 and [177Lu]TXP02-JN005 Heart (blood) uptake at 4, 24 and 48 hours

| | | | Dose | 4 hrs: | 24 hrs: | 48 hrs: |
|---|---|---|---|---|---|---|
| | | | Radioactivity | blood | blood | blood |
| | Mass dose | | (Mbq/animal, | % ID/g | % ID/g | % ID/g |
| Group | (μg/animal) | Imaging tracer | Mean ± SD) | (Mean ± SD) | (Mean ± SD) | (Mean ± SD) |
| 1 | 100 | [¹⁷⁷Lu] TXP02- | 19.4 ± 0.6 | 11.7 ± 1.7 | 3.0 ± 0.2 | 1.7 ± 0.1 |
| | | JN007 | | | | |
| 2 | 100 | [¹⁷⁷Lu] TXP02- | 19.7 ± 0.7 | 16.5 ± 1.1 | 10.0 ± 0.6 | 7.8 ± 0.9 |
| | | JN005 | | | | |

FIG. 14 is a plot of the tumour:blood ratios in mice following administration the JN007 antibody (anti-PSMA, K-DOTA-Lu antibody of the invention modified to reduce FcRn-binding—also referred to as HuX592R-DOTA-Lu177).

Compared to control mice (which received JN005, i.e., anti-PSMA, K-DOTA-Lu antibody HuJ591-DOTA-Lu177 which does not have modifications in the FcRn-binding region), mice that received the FcRn modified antibody had a higher ratio of antibody in tumour compared to blood.

### Example 8: In Vivo Evaluation of PET Imaging Agents

### Test Antibodies

FcRn-GmAb=⁸⁹Zr-DFOsq-FcRn-GmAb (an anti-CAIX-binding antibody of the invention modified to reduce FcRn binding, labelled with zirconium).

cGmAb-CHO=⁸⁹Zr-DFOsq-cGmAb-CHO (an anti-CAIX-binding antibody of the invention that does not comprise a modification to reduce FcRn binding, labelled with zirconium).

### Experimental Methods

Female Balb/c mice (Australian BioResources, NSW, 8 weeks) were inoculated subcutaneously on the right flank with 5×10⁶ HT-29 cells in 50% Matrigel;
nine HT-29 tumour bearing mice were injected intravenously with 3 MBq FcRn-GmAb or 3 MBq cGmAb-CHO, respectively in a volume of 120-130 μl;
at 4, 24, 48 and 144 post injection three mice were anaesthetised using isoflurane and placed on the imaging bed of a G8 PET/CT scanner (Perkin Elmer);
a CT scan was performed and followed immediately by a 10 min static PET scan;
PET images were reconstructed using the maximal likelihood and expectation maximization (ML-EM) algorithm and analysed using VivoQuant (Invicro). Tumour SUVmax, Tumour SUVmax/background average (TBR), where background represents mediastinal uptake, Tumour SUVmax/liver average (Tumour:Liver) and Tumour SUVmax/bone average (Tumour:Bone), where bone represents the region of high uptake around the joint between the fibula and tibia, were determined.
a cohort of three animals was euthanised at 24, 48 and after imaging at 144 hr and selected tissues were removed, weighed and counted in a gamma counter. Tracer uptake was calculated as % injected dose/gram tissue.
Statistical analysis of the data was performed using GraphPad Prism 7.

### Results

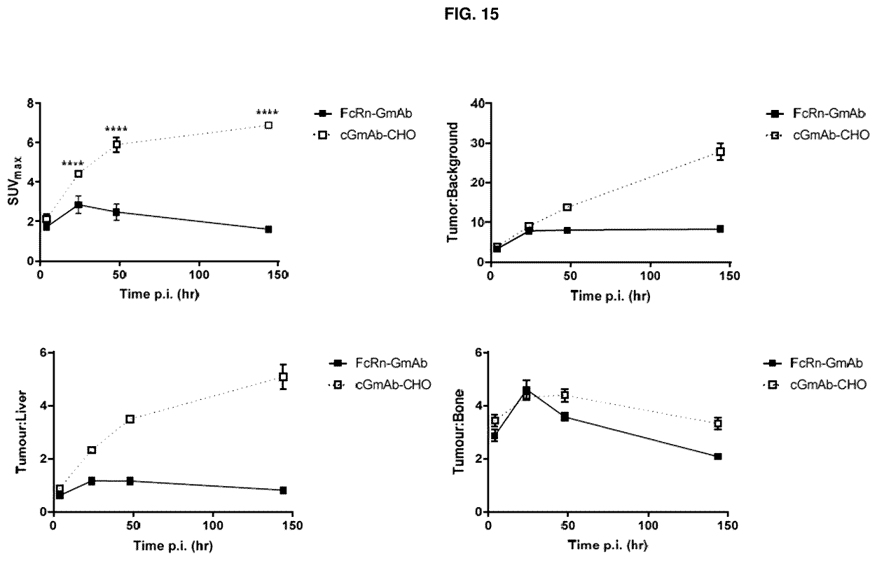

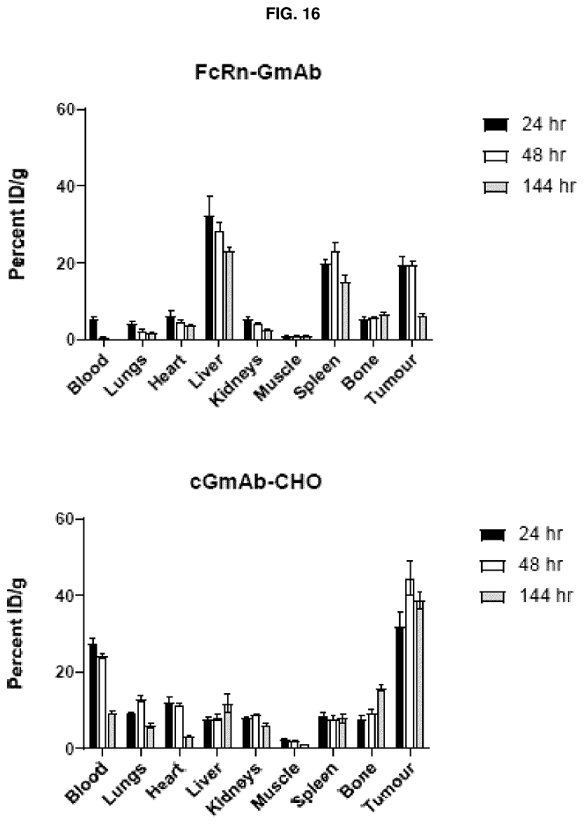

The PET images of mice injected with FcRn-GmAb and cGmAb-CHO were obtained and quantitation of the PET images is shown in FIG. 15. The tissue biodistribution data for each tracer is summarised in FIG. 16.

The FcRn-GmAb PET images show substantial liver accumulation at 4 hr p.i. and modest tumour uptake at 144 hr p.i. In contrast, the cGmAb-CHO images show minimal liver accumulation and high tumour uptake at 144 hr p.i. Analysis of the PET images shows tumour uptake of cGmAb-CHO increased linearly from 4 to 48 hr, after which levels began to plateau (FIG. 15, top left panel). Maximal tumour uptake of FcRn-GmAb was seen at 24 hr with tracer washout evident at 144 hr. cGmAb-CHO tumour SUVmax was significantly higher than that of FcRn-GmAb at 24, 48 and 144 hr p.i. (P<0.0001, Sidak's multiple comparisons test).

The cGmAb-CHO tumour to background ratio (TBR) increased linearly between 4 and 144 hours while FcRn-GmAb TBR increased from 4 hours to 24 hours then plateaued until 144 hours (FIG. 15, top right panel). The cGmAb-CHO tumour to liver ratio (TLR) increased linearly to 48 hr after which it plateaued while FcRn-GmAb TLR increased to 24 hr before declining to 4 hr levels at 144 hr (FIG. 15, bottom left panel).

Consistent with the PET imaging results, the biodistribution data (FIG. 16) show (i) slower washout of cGmAb-CHO from blood, lungs, kidneys, muscle, bone and tumour compared with FcRn-GmAb; (ii) lower liver and spleen uptake of cGmAb-CHO than FcRn-GmAb at 24, 48 and 144 hours; (iii) tumour uptake and retention of cGmAb-CHO was significantly higher than FcRn-GmAb at 48 (P=0.006, t-test) and 144 hours (P=0.0002, t-test). No analysis was performed at 24 hr as n=2 in cGmAb-CHO group.

### Example 9: Therapeutic Efficacy of an Exemplary Antibody of the Invention

### Test Antibodies

1. HuX592R (ANT4044-FcRN).
2. HuJ591 (ANT4044)

### Radiolabelling and TLC Analysis of Antibodies

All antibodies were incubated with ¹⁷⁷Lu at a 50-fold or 100-fold excess of biomolecule for HuX592R and HuJ591 respectively in 0.1 M pH 5.5 ammonium acetate buffer for 45 minutes at room temperature. Samples of each solution were taken and mixed 1:1 with 50 mM DTPA. 5 uL of each DTPA incubated sample or neat solution was spotted on TLC paper (Agilent iTLC-SG Glass microfiber chromatography paper impregnated with silica gel) and run with 50:50 H₂O:ethanol. Detection of radiolabelled species migration was then achieved using an Eckert and Ziegler Mini-Scan and Flow-Count system. All samples showed 90% labelling. Control experiments were conducted to monitor the elution behaviour of free ¹⁷⁷Lu and ¹⁷⁷Lu bound to DTPA for quality control.

### Cell Binding

Lu-labelled constructs HuX592R and HuJ591 were assessed for cell binding.

In summary, Eppendorf tubes containing 1.25×10⁵ PC-3 tumour cells (negative control) or 1.25×10⁵ LNCaP tumour cells in 0.100 mL PBS are incubated at 37° C. with 5 μL (0.030 MBq) of labelled antibody. The incubations were stopped at the following time points for analysis: 1 hour, 2 hours, 4 hours and 24 hours.

### Unbound Fraction Determination.

At the end of each incubation period, the Eppendorf tubes are centrifuged for 5 min at 500 g. The supernatants containing the free Lu-177 antibody were recovered in separate tubes and counted using gamma analysis. The pellets containing the cells associated with Lu-177 labelled antibody were washed with 0.200 mL of PBS solution and centrifuged for 5 min at 500 g for 3 repeats. Supernatants of each wash were collected and counted, values combined with the recovered incubation supernatant to result a total free unbound antibody.

### Surface Bound Fraction Determination.

Pellets were resuspended in 0.1 mL PBS at pH 4.0 for 20 min at ice cold temperature. Eppendorf tubes were then centrifuged for 5 min at 500 g at 4° C. and supernatants collected for counting. Following a further three washes with ice cold PBS pH 4.0, the supernatants were collected for gamma counting.

### Internalised Fraction Determination.

After washing and centrifugation, the cell pellets were counted as internalised fraction.

### Protein Concentration

Cells were solubilised by 1 M NaOH and protein concentration determined.

### Calculation

The binding of Lu-177 antibody to the tumour cells (surface bound and internalised fractions) and unbound antibodies were calculated and reported as a percentage of the total radioactivity incubated per mg of protein.

### Animals

Healthy male Balb/C nude mice (˜20 g) from 6 weeks old were obtained from the ARC (Western Australia) and used for this study. Mice were monitored for 1 week prior to the study in order to acclimatise to the environment prior to injection of cells. All animals were provided with free access to food and water before and during the experiments.

### Dosing

The injection syringes were filled with the radiolabelled antibody solution (approximately 100 μL) and the activity in the syringe was measured using a dose calibrator (Capintec CRC-25) with a calibration factor of 35. The activity left in the syringe after the tail vein injection was measured using the same dose calibrator and the total volume injected in each mouse was calculated.

### Tolerability

Healthy male Balb/C nude mice (n=3 per dosing cohort) were injected (29 G, tail vein injection in ˜100 μL saline) a total of 3 times 7 days apart with ¹⁷⁷Lu-labelled HuX592R to give 6, 9 or 12 MBq injected dose at a mass dose of approximately 100 μg per mouse. Mouse health was then monitored following injection, and at 28 days post initial injection all mice were culled and organs fixed in PFA, transferred to 30% sucrose and allowed to decay before being stored frozen for potential future analysis if required.

### Biodistribution

Healthy male Balb/C nude mice (n=3 per cohort) were injected (29 G, tail vein injection saline) with ¹⁷⁷Lu-labelled constructs to give 6 MBq injected dose at a mass dose of 100 (HuX592R) or 140 (HuJ591) μg per mouse. Animals were then sacrificed at 24, 48 and 72 hours post injection (for HuX592R) and 72 hrs (for HuJ591). Blood was sampled and tissues collected and cleaned of excess blood and weighed for ex vivo analysis. A Perkin PerkinElmer 2480 Automatic Gamma Counter was used to measure radioactivity in tissues. The gamma counter was calibrated using known samples of ¹⁷⁷Lu and measured activity presented as % ID/g of tissue weight based on injected activities.

### Tumour Initiation and Growth

For the autoradiography and therapeutic studies, 8-12 week old male Balb/c nude mice were injected (27G needle) subcutaneously with 4×10⁶ LNCaP cells in 50 μL 50:50 phosphate buffered saline:matrigel into the right flank of each mouse. There was no evidence of ulceration at the time of cell injection; the animals were closely monitored and tumour measured by callipers and remained in good condition apart from the growth of solid tumours. The tumour growth was sporadic as is often observed for LNCaP tumours and mice were enrolled in appropriate study arms as tumours reached 100-200 mm³.

### Autoradiography

Tumour bearing male Balb/C nude mice (n=2 per cohort) were injected (29 G, tail vein injection in ˜100 μL saline) with ¹⁷⁷Lu-labelled constructs to give 6 MBq injected dose at a mass dose of 100 (HuX592R) or 140 (HuJ591) μg per mouse. Animals were then sacrificed at 7 days post injection, tumour samples were snap frozen in an isopentane-dry ice slurry, then embedded in OCT compound for sectioning. 20 μm sections were collected onto slides and air dried, then exposed on a phosphor screen in a closed cassette for approximately 3.5 hours. Images were then obtained using an Amersham Typhoon Phosphorimager using a pixel size of 50 μm (scan time 20-30 min) as optimized for ¹⁷⁷Lu with a sensitivity setting of 1000. ImageQuant TL software was used to analyze images.

### Therapeutic Study

Tumour bearing male Balb/C nude mice (n=6 per cohort) were injected (29 G, tail vein injection in ˜100 μL saline) with ¹⁷⁷Lu-labelled constructs to give 6 or 8 MBq for HuX592R or 6 MBq for HuJ591 injected dose at a mass dose of 100 for HuX592R or 140 for HuJ591 μg per mouse each week for 3 weeks. Tumour growth in response to therapy was monitored over 90 days in comparison to vehicle only control.

### Results

Both HuX592R and HuJ591 showed successful ¹⁷⁷Lu labelling. These ¹⁷⁷Lu labelled constructs were used directly for the cell binding, tolerability, biodistribution and therapeutic efficacy studies.

### Cell Binding

Binding of ¹⁷⁷Lu-labelled HuX592R and HuJ591 was assessed against LNCaP (PSMA+) and PC3 (PSMA−) cell lines. The unbound, surface bound and cell pellet associated fractions were assessed at 1, 2 and 4 hours after addition of the constructs to the cells. The cell assay showed that both HuJ591 and HuX592R showed enhanced accumulation in PSMA expressing cell lines, with the highest concentration observed in the cell pellet indicating successful internalisation during the assay period (at all timepoints). Minimal association/internalisation was generally observed in the PSMA-negative cell line (PC3), indicating that binding was receptor-dependent and the labelling did not interfere significantly with the antibody binding to the receptor.

### Tolerability

Tolerability of ¹⁷⁷Lu-labelled HuX592R in healthy male Balb/c nude mice (n=3 per cohort) was assessed at 3 dosing levels, 12, 9, and 6 MBq administered 3 times one week apart. Animal health was assessed by animal weight and animal score sheet for 4 weeks including 3 weeks of therapeutic administration. In this study, it was found that the 12 MBq dose too high, so the full study was then undertaken using doses of 6 MBq and 8 MBq.

### Biodistribution

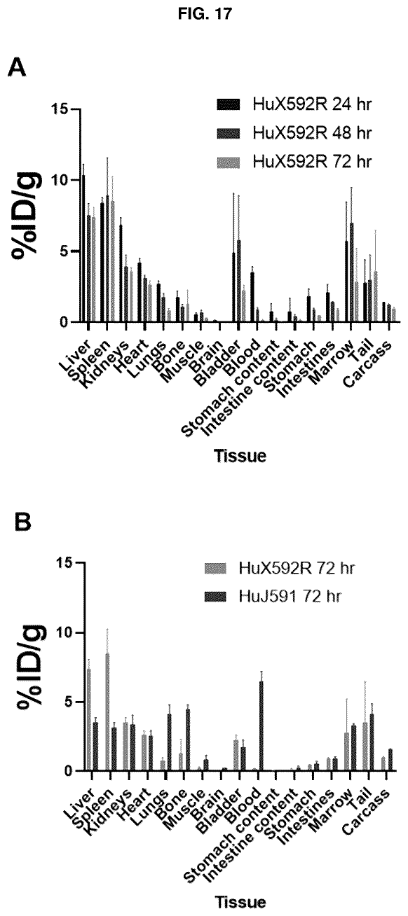

Biodistribution of ¹⁷⁷Lu-labelled constructs in healthy male Balb/c nude mice (n=3 per cohort) was assessed at 24, 48 and 72 hrs post administration for HuX592R (FIG. 17A) and at 72 hrs only post administration for HuJ591 (FIG. 17B). The results show significantly higher circulation time for HuJ591 compared to HuX592R with commensurate lower levels of accumulation in clearance organs such as the liver and spleen suggesting more rapid clearance by the system.

### Autoradiography

¹⁷⁷Lu-labelled constructs were administered into tumour-bearing male Balb/c nude mice. Animals were sacrificed 1 week post injection, tumours frozen in OCT, sectioned and autoradiography images acquired. Overall results are in agreement with tumour accumulation and penetration by both constructs, with enhanced accumulation for HuJ591 compared to HuX592R at this timepoint. Moreover, distribution of the radiotherapeutic appears to be well-dispersed throughout the tumour for both antibodies.

### Therapeutic Study

Male Balb/c nude mice bearing flank LNCaP xenograft tumours were assigned to therapeutic cohorts and administered 3 doses 1 week apart of either,
1. Vehicle control
2. 6 MBq [¹⁷⁷Lu]-HuX592R
3. 8 MBq [¹⁷⁷Lu]-HuX592R
4. 6 MBq [¹⁷⁷Lu]-HuJ591

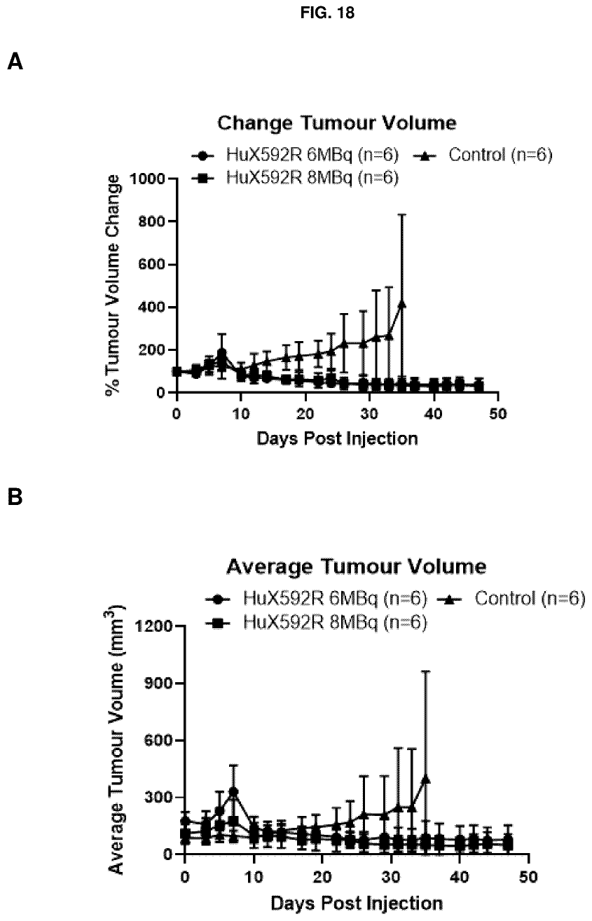

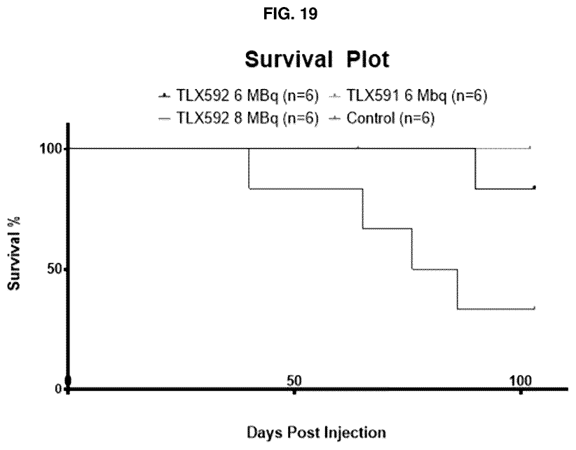

Tumour regression (FIG. 19) and animal health as assessed by animal weight was monitored for 100 days. Results show significant tumour growth inhibition for HuX592R (FIG. 18) with 2 mice in the HuX592R 8 MBq and 4 mice in the HuJ591 (not shown) cohort showing complete tumour regression where tumour burden was not measurable by callipers. At 37 days after commencing therapy (the last datapoint at which all cohorts were n=6 allowing statistical comparison) all 3 treatments produced significant reductions in tumour growth compared to the control cohort as determined by 2 way ANOVA, with HuX592R at 6 MBq (p 0.0105), HuX592R at 8 MBq (p 0.0086) and HuJ591 at 6 MBq (p 0.0056).

No dosing group showing significant ill-health as assessed by change in body weight.

Mouse survival (FIG. 19) is statistically longer (p<0.05 by Mantel-Cox test) for all 3 treatment cohorts (median survival >study period) compared to the control cohort (median survival 83.5 days). Upward tick indicates animal euthanised due to non-tumour/health related issues.

Both formulations (HuJ591 at 6 MBq) and HuX592R (at both 6 MBq and 8 MBq) showed efficacy against PSMA expressing tumours. Both HuJ591 (at 6 MBq) and HuX592R (at 8 MBq) had no treatment/tumour related deaths during the 100 days of the study, and 2 mice in the HuX592R 8 MBq and 4 mice in the HuJ591 groups showed absolute regression of the tumour (immeasurable by calipers). In terms of the survival plot, all treatment groups with antibody were statistically better than the control group.

### Example 10: Biodistribution, Therapeutic Efficacy and Tolerability of ²²⁵-Ac-DOTA- and ¹⁷⁷-Lu-DOTA-Labelled Anti-CAIX Binding Antibodies of the Invention

An example of a humanised antibody for binding CAIX (e.g., comprising the amino acid sequence as set out in Example 1) was labelled with either an alpha-particle-emitting radioisotope (actinium 225) or beta-particle emitting radioisotope (lutetium 177). Radiolabelling was performed using standard conjugation methods as described herein (i.e., using a DOTA chelating agent).

Balc/c nude mice bearing SK-RC-52 xenografts were assigned into 4 treatment groups and 1 control group as follows:
1. ²²⁵Ac-hG250 5 kBq
2. ²²⁵Ac-hG250 15 kBq
3. ²²⁵Ac-hG250 25 kBq
4. ¹⁷⁷Lu-hG250 13 MBq
5. Control

N=10 per group.

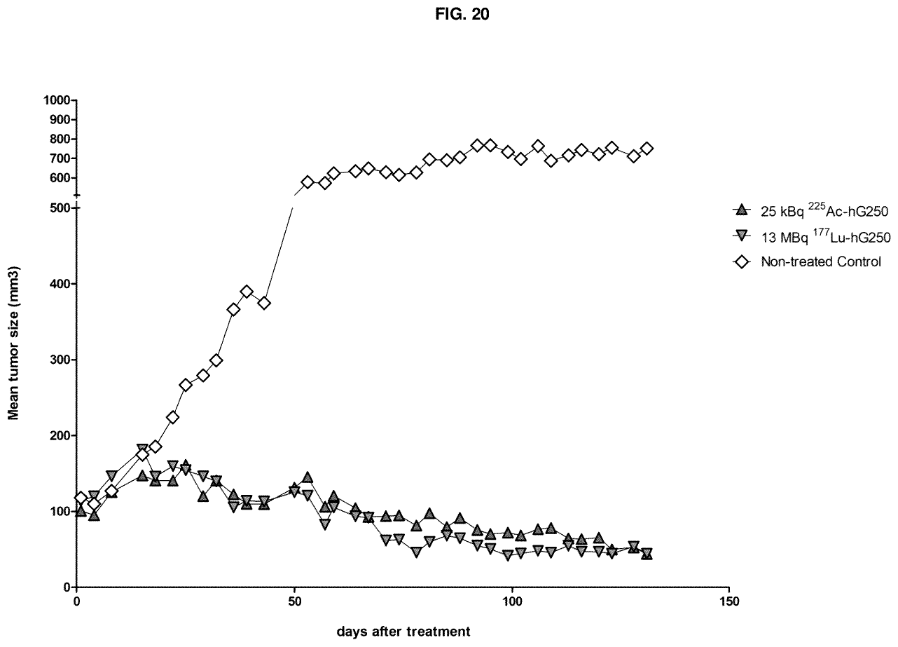

Tumour growth rate and tumour size were assessed for each group. FIG. 20 shows the mean tumour size (mm³) over the period of time after treatment. The results show a rapid and sustained reduction in tumour size over time, compared to non-treatment control.

It will be understood that the invention disclosed and defined in this specification extends to all alternative combinations of two or more of the individual features mentioned or evident from the text or drawings. All of these different combinations constitute various alternative aspects of the invention.

1-74. (canceled)

75. An antigen binding protein comprising an antigen binding domain of an antibody, wherein the antigen binding domain binds to or specifically binds to CAIX, wherein the antigen binding domain comprises:
a VH comprising a complementarity determining region (CDR) 1 comprising a sequence set forth in SEQ ID NO 65, a CDR2 comprising a sequence set forth in SEQ ID NO: 66, and a CDR3 comprising a sequence set forth in SEQ ID NO: 67;
a VL comprising a CDR1 comprising a sequence set forth in SEQ ID NO: 145, a CDR2 comprising a sequence set forth in SEQ ID NO: 146, and a CDR3 comprising a sequence set forth in SEQ ID NO: 147;
a VH comprising a framework region (FR) 1 comprising a sequence at least about 80% identical to a sequence set forth in SEQ ID NO: 73, 89, 105, or 124, a FR2 comprising a sequence at least about 80% identical to a sequence set in SEQ ID NO: 74, 90, 106, or 122, a FR3 comprising a sequence at least about 80% identical to a sequence set forth in SEQ ID NO: 75, 91, 107, or 123, and a FR4 comprising a sequence at least about 80% identical to a sequence set forth in SEQ ID NO: 76, 92, 108, or 124; and
a VL comprising a FR1 comprising a sequence at least about 80% identical to a sequence set forth in SEQ ID NO: 153, 169, 185, 201, or 217, a FR2 comprising a sequence at least about 80% identical to a sequence set forth in SEQ ID NO: 154, 170, 186, 202, or 218, a FR3 comprising a sequence at least about 80% identical to a sequence set forth in SEQ ID NO: 155, 171, 187, 203, or 219, and a FR4 comprising a sequence at least about 80% identical to a sequence set forth in SEQ ID NO: 156, 172, 188, 204, or 220.

76. The antigen binding protein of claim 75, wherein the antigen binding domain comprises:
a VH comprising a framework region (FR) 1 comprising a sequence a sequence set forth in SEQ ID NO: 73, 89, 105, or 124, a FR2 comprising a sequence set in SEQ ID NO: 74, 90, 106, or 122, a FR3 comprising a sequence set forth in SEQ ID NO: 75, 91, 107, or 123, and a FR4 comprising a sequence set forth in SEQ ID NO: 76, 92, 108, or 124; and
a VL comprising a FR1 comprising a set forth in SEQ ID NO: 153, 169, 185, 201, or 217, a FR2 comprising a sequence set forth in SEQ ID NO: 154, 170, 186, 202, or 218, a FR3 comprising a sequence set forth in SEQ ID NO: 155, 171, 187, 203, or 219, and a FR4 comprising a sequence set forth in SEQ ID NO: 156, 172, 188, 204, or 220.

77. The antigen binding protein of claim 75, wherein the antigen binding domain comprises:
a VH comprising a sequence at least about 80% identical to a sequence set forth in SEQ ID NO: 68, 84, 100 or 116; and
a VL comprising a sequence at least about 80% identical to a sequence set forth in SEQ ID NO: 148, 164, 180, 196 or 212.

78. The antigen binding protein of claim 75, wherein the antigen binding domain comprises:
a VH comprising a sequence set forth in SEQ ID NO: 68, 84, 100 or 116; and
a VL comprising a sequence set forth in SEQ ID NO: 148, 164, 180, 196 or 212.

79. The antigen binding protein of claim 75, wherein the antigen binding domain comprises:
(i) a VH comprising a sequence at least about 80% identical to a sequence set forth in SEQ ID NO: 84, and a VL comprising a sequence at least about 80% identical to a sequence set forth in SEQ ID NO: 164;
(ii) a VH comprising a sequence at least about 80% identical to a sequence set forth in SEQ ID NO: 84, and a VL comprising a sequence at least about 80% identical to a sequence set forth in SEQ ID NO: 180;
(iii) a VH comprising a sequence at least about 80% identical to a sequence set forth in SEQ ID NO: 84, and a VL comprising a sequence at least about 80% identical to a sequence set forth in SEQ ID NO: 196;
(iv) a VH comprising a sequence at least about 80% identical to a sequence set forth in SEQ ID NO: 100, and a VL comprising a sequence at least about 80% identical to a sequence set forth in SEQ ID NO: 164;
(v) a VH comprising a sequence at least about 80% identical to a sequence set forth in SEQ ID NO: 100, and a VL comprising a sequence at least about 80% identical to a sequence set forth in SEQ ID NO: 180; or
(vi) a VH comprising a sequence at least about 80% identical to a sequence set forth in SEQ ID NO: 100, and a VL comprising a sequence at least about 80% identical to a sequence set forth in SEQ ID NO: 196.

80. The antigen binding protein of claim 75, wherein the antigen binding domain comprises:
(i) a VH comprising a sequence set forth in SEQ ID NO: 84, and a VL comprising a sequence set forth in SEQ ID NO: 164;
(ii) a VH comprising a sequence set forth in SEQ ID NO: 84, and a VL comprising a sequence set forth in SEQ ID NO: 180;
(iii) a VH comprising a sequence set forth in SEQ ID NO: 84, and a VL comprising a sequence set forth in SEQ ID NO: 196;
(iv) a VH comprising a sequence set forth in SEQ ID NO: 100, and a VL comprising a sequence set forth in SEQ ID NO: 164;
(v) a VH comprising a sequence set forth in SEQ ID NO: 100, and a VL comprising a sequence set forth in SEQ ID NO: 180; or
(vi) a VH comprising a sequence set forth in SEQ ID NO: 100, and a VL comprising a sequence set forth in SEQ ID NO: 196.

81. The antigen binding protein of claim 75, wherein the antigen binding protein is an antibody.

82. The antigen binding protein of claim 75, wherein the antigen binding protein is a naked antibody.

83. The antigen binding protein of claim 75, wherein the antigen binding protein is an antibody conjugated to a label, including a radiolabel or a cytotoxic agent.

84. The antigen binding protein of claim 75, wherein the antigen binding protein is an IgG immunoglobulin comprising one or more amino acid substitutions in the antibody constant domain, CH2-CH3 region, which reduce the binding affinity of the antibody to the neonatal Fc receptor (FcRn) relative to a wild-type antibody Fc region.

85. The antigen binding protein of claim 84, wherein the IgG immunoglobulin comprises one or more amino acid substitutions at positions His310, His435, Ile253 of the heavy chain constant region compared to wild-type IgG immunoglobulin.

86. The antigen binding protein of claim 75, wherein the antigen binding protein is an IgG immunoglobulin comprising one or more amino acid substitutions which modify the binding of the antibody to activating Fc gamma receptors, such as wherein the amino acid modification is from Leu235 to glutamic acid; and/or
wherein the antigen binding protein comprises a hinge stabilising amino acid modification at Ser228, wherein preferably the amino acid modification at Ser228 is to proline.

87. A pharmaceutical composition comprising an antigen binding protein of claim 75.

88. An in vivo method of diagnosing, monitoring or prognosing a disease, disorder or infection in a subject comprising:
(a) administering to a subject an effective amount of the antigen binding protein of claim 75, said antigen binding protein specifically binding to an antigen associated with a disease, disorder or infection;
(b) allowing the antigen binding protein to concentrate at sites in said subject where said antigen is found; and
(c) detecting said antigen binding protein;
whereby detection of said antigen binding protein above a background or standard level indicates that the subject has said disease disorder or infection.

89. The method of claim 88, wherein the antigen binding protein is conjugated to a radiolabel.

90. The method of claim 89, wherein the radiolabel is selected from the group consisting of: actinium-225 (²²⁵Ac), astatine-211 (²¹¹At), bismuth-212 and bismuth-213 (²¹²Bi, ²¹³Bi), copper-64 and copper-67 (⁶⁴Cu, ⁶⁷Cu), gallium-67 and gallium-68 (⁶⁷Ga and ⁶⁸Ga), indium-111 (¹¹¹In), iodine-123, -124, -125 or -131 (¹²³I, ¹²⁴I, ¹²⁵I, ¹³¹I) preferably (124 I), lead-212 (²¹²Pb), lutetium-177 (¹⁷⁷Lu), radium-223 (²²³Ra), samarium-153 (¹⁵³Sm), scandium-44 and scandium-47 (⁴⁴Sc, ⁴⁷Sc), strontium-90 (⁹⁰Sr), technetium-99 (^{99m}Tc), yttrium-86 and yttrium-90 (⁸⁶Y, ⁹⁰Y), and zirconium-89 (⁸⁹Zr).

91. The method of claim 89, wherein the radiolabel is selected from: gallium-67 and gallium-68 (⁶⁷Ga and ⁶⁸Ga), iodine-124 (¹²⁴I) and zirconium-89 (⁸⁹Zr).

92. A method of treating cancer in a subject in need thereof, wherein the method comprises administering to the subject a therapeutically effective amount of an antigen binding protein of claim 75 or a pharmaceutical composition comprising the same.

93. The method of claim 92, wherein the antigen binding protein is conjugated to a radiolabel.

94. The method of claim 93, wherein the radiolabel is selected from the group consisting of: actinium-225 (²²⁵Ac), astatine-211 (²¹¹At), bismuth-212 and bismuth-213 (²¹²Bi, ²¹³Bi), copper-64 and copper-67 (⁶⁴Cu, ⁶⁷Cu), gallium-67 and gallium-68 (⁶⁷Ga and ⁶⁸Ga), indium-111 (¹¹¹In), iodine-123, -124, -125 or -131 (¹²³I, ¹²⁴, ¹²⁵I, ¹³¹I) preferably (124 I), lead-212 (²¹²Pb), lutetium-177 (¹⁷⁷Lu), radium-223 (²²³Ra), samarium-153 (¹⁵³Sm), scandium-44 and scandium-47 (⁴⁴Sc, ⁴⁷Sc), strontium-90 (⁹⁰Sr), technetium-99 (^{99m}Tc), yttrium-86 and yttrium-90 (⁸⁶Y, ⁹⁰Y), and zirconium-89 (⁸⁹Zr).

95. The method of claim 93, wherein the radiolabel is selected from: actinium-225 (²²⁵Ac), astatine-211 (²¹¹At) and lutetium-177 (¹⁷⁷Lu).
